(19) United States
(12) Patent Application Publication
(10) Pub. No.: US 2026/0262965 A1
(43) Pub. Date: Sep. 10, 2026

(54) SYSTEMS AND METHODS FOR DETECTION AND/OR PREDICTION OF FALL RISKS AND COGNITIVE IMPAIRMENT

(71) Applicant: Florida Atlantic University Board of Trustees, Boca Raton, FL (US)

(72) Inventors: Behnaz Ghoraani, Boca Raton, FL (US); Mahmoud Seifallahi, Boca Raton, FL (US)

(21) Appl. No.: 19/426,926

(22) Filed: Dec. 19, 2025

Related U.S. Application Data
(60) Provisional application No. 63/767,842, filed on Mar. 6, 2025.

(51) Int. Cl. A61B 5/11; A61B 5/00

(52) U.S. Cl. CPC A61B 5/1117; A61B 5/112; A61B 5/1128; A61B 5/4088; A61B 5/4839; A61B 5/7271; A61B 5/742

(57) ABSTRACT

Systems and methods for detection and/or prediction of fall risks and cognitive impairment. The methods comprise: capturing, by a camera, a video of a subject performing an activity or task with a cognitive load and without the cognitive load; processing, by a processor, the video to generate a data object for the subject; processing, by a processor or a machine learning model, the data object to determine gait features and balance features for the subject; performing, by the processor or machine learning model, operations to detect or predict a fall risk and/or cognitive impairment for the subject based on at least the gait features and balance features; and/or determining, by the processor, a diagnosis for the subject based on the detected or predicted fall risk and/or cognitive impairment.

### CROSS-REFERENCE TO RELATED APPLICATIONS

The present application claims priority to and the benefit of U.S. Provisional Patent Application No. 63/767,842 which was filed on Mar. 6, 2025. The content of this U.S. Provisional Patent Application is incorporated herein by reference in its entirety.

### GOVERNMENT SUPPORT STATEMENT

This invention was made with government support under Grant no. 1942669 awarded by the National Science Foundation and Grant no. CNS-1950400 awarded by the National Science Foundation Research Experiences for Undergraduates. The government has certain rights in the invention.

### BACKGROUND

Current diagnostic methods for cognitive impairments and Alzheimer's disease (AD), such as neuropsychological assessments, brain imaging, and blood tests, are often expensive, time-consuming, and require specialized medical facilities and expertise. These methods also carry the risk of late diagnosis and are inaccessible to many due to their high costs and the need for specialized equipment.

### SUMMARY

The present document concerns implementing systems and methods for detection and/or prediction of fall risks and cognitive impairment. The methods comprise: capturing, by a camera, a video of a subject performing an activity or task with a cognitive load and without the cognitive load; processing, by a processor, the video to generate a data object for the subject; processing, by a processor or a machine learning model, the data object to determine gait features and balance features for the subject; performing, by the processor or machine learning model, operations to detect or predict a fall risk and/or cognitive impairment for the subject based on at least the gait features and balance features; and/or determining, by the processor, a diagnosis for the subject based on the detected or predicted fall risk and/or cognitive impairment.

The present document also concerns a system comprising a camera and a processor. The camera is configured to capture a video of a subject performing activity or task with a cognitive load and without the cognitive load. The processor is configured to: process the video to generate a data object for the subject; process the data object to determine gait features and balance features for the subject; perform operations to detect or predict a fall risk and/or cognitive impairment for the subject based on at least the gait features and balance features; and/or determine a diagnosis for the subject based on the detected or predicted fall risk and/or cognitive impairment.

### BRIEF DESCRIPTION OF DRAWINGS

The present solution will be described with reference to the following drawing figures, in which like numerals represent like items throughout the figures.

FIG. 1A provides an illustration of a system in accordance with the present solution. FIGS. 1B-1E provide drawings that are useful for understanding an illustrative use case for the system shown in FIG. 1A. FIGS. 1A-2E are collectively referred to as “FIG. 1”.

FIGS. 2A-2B (collectively referred to as “FIG. 2”) provides a flow diagram of an illustrative method for diagnosing a subject with a cognitive impairment or disorder.

FIG. 3 provides an illustration showing possible joints that may be detected by a system implementing the present solution.

FIG. 4 provides an illustration showing a system set-up for an experiment.

FIG. 5 provides an illustration that is useful for understanding a process that was performed during an experiment for health control (HC) or AD detection.

FIGS. 6A-6B (collectively referred to as “FIG. 6”) provide images showing cognitive impairment testing and a comparative analysis of gait performance between AD and HC. FIG. 6A shows the open-source algorithm's performance with clear visibility, accurately tracking body joints and segments during unobstructed walking sequences. FIG. 6B shows challenges in the open-source algorithm's detection due to full occlusion of body parts, illustrating missed joints and segments in obscured walking frames.

FIGS. 7A-7C (collectively referred to as “FIG. 7”) provide graphs providing a comparative analysis of gait performance between AD and HC, showcasing FIG. 7A—the distribution of extracted, significant, and selected gait features; FIG. 7B—a correlation heatmap for identifying unique selected gait features; and FIG. 7C—a comparison of macro and micro gait features between the two groups.

FIGS. 8A-8D (collectively referred to as “FIG. 8”) show recording tools and sample data. FIG. 8A shows a camera connected to a laptop. FIG. 8B shows trackable joints of the body. FIG. 8C shows RGB and skeletal data for straight path walking. FIG. 8D shows RGB and skeletal data for oval path walking.

FIG. 9 shows general steps of the proposed algorithm for processing and analysis of recorded skeletal data for gait tests to detect MCI.

FIGS. 10A-10B (collectively referred to as “FIG. 10”) show feature extraction using right and left foot signals and their distance. FIG. 10A provides a graph showing the detection of the subphases of gait cycles using the stability or changes in the locations of feet. FIG. 10B provides a graph showing the detection of gait cycles and steps using distance signal of feet.

FIGS. 11A-11B (collectively referred to as “FIG. 11”) provide graphs showing comparison of the gait between two study groups in different conditions. The graph of FIG. 11A shows the comparison of average velocity and the velocity of steps and strides for MCI vs. HC. The graph of FIG. 11B shows the comparison of the variability of gait cycle subphases for MCI vs. HC. *Significant difference at p-value<0.05.

FIGS. 12A-12B (collectively referred to as “FIG. 12”) provide graphs showing correlation heatmaps of significant features for straight and oval path walking. The graph of FIG. 12A shows a correlation heatmap of significant features for MCI vs. HC during straight walking. FIG. 12B shows a correlation heatmap of significant features for MCI vs. HC during oval path walking.

FIGS. 13A-13B (collectively referred to as “FIG. 13”) provides graphs showing an analysis of the importance of features using the RF classifier. The purple dash line shows the threshold of 0.03 for the scores of the importance of the features provided by RF classifier. The graph of FIG. 13A ranks the importance of straight walking features for discrimination of MCI and HC. The graph of FIG. 13B ranks the importance of oval path walking features for discrimination of MCI and HC.

FIGS. 14A-14B (collectively referred to as “FIG. 14”) provide illustrations for another scenario using the recording tools and setup of FIG. 8A and the map of joints that can be tracked by the camera shown in FIG. 8B. FIG. 14A shows a participant performing curve walking beside the camera that records movement signals from 25 body joints. FIG. 14B shows a subject performing straight walking and recorded signals of his body joints in the form of skeleton by the camera.

FIGS. 15A-15D (collectively referred to as “FIG. 15”) provides graphs showing comparison of gait markers between two study groups in straight and curve walking conditions.

FIGS. 16A-16D (collectively referred to as “FIG. 16”) provides graphs showing an effect of changing from straight to curve walking on the performance of MCI and HC participants.

FIGS. 17A-17B (collectively referred to as “FIG. 17”) provides graphs showing a comparison of gait markers between two study groups in various areas of curved path walking. This figure illustrates the comparative analysis of various gait markers between the two study groups across distinct sections of the curved path, labeled as sections A1 through A8. Each section represents a specific segment along the curved trajectory. * shows the significant difference for p-value<0.05 and ‡ shows the significant difference for p-value<0.005.

FIG. 18 provides a graph showing receiver operating characteristic (ROC) curves comparing the diagnostic performance of curved walking (CW) and straight walking (SW) in discriminating between mild cognitive impairment (MCI) and HC participants. AUC: area under the curve.

FIG. 19 provides a block diagram of a computing device.

FIG. 20 provides graphs showing raw gait signals and synchronized trigger signals across the three sensor systems for a single subject.

FIG. 21A provides a graph showing heel strike and toe-off events on the foot contact data for a representative pressure sensitive mat. FIG. 21B provides a graph showing heel strike and toe-off events for foot sensors. FIG. 21C provides a graph showing heel strike and toe-off events for a lumbar sensor. FIG. 21D provides a graph showing heel strike and toe-off events for an acceleration sensor. FIGS. 21A-21D are collectively referred to as “FIG. 21”.

FIG. 22 provides a graph showing local maximum and minimum of ankle acceleration signals.

### DETAILED DESCRIPTION

AD diagnostic methodologies (e.g., brain imaging, Electroencephalography, and blood/neuropsychological tests) are not only expensive and protracted but also require specialized medical expertise. Addressing these constraints, the present solution provides a cost-efficient and universally accessible system to detect early signs of cognitive impairment, AD and related disorders through the analysis of human gait patterns. The present solution uses standard video cameras (e.g., smart phones) to capture footage of an individual walking along a predetermined path (e.g., a curved, figure-of-eight, or straight line path) and applies advanced computer vision and machine learning algorithms to analyze the data. The present solution simplifies the detection process by using readily available and inexpensive standard cameras, making it accessible for use in clinical and non-clinical settings (e.g., homes or community centers). This approach significantly reduces the cost and logistical barriers associated with other diagnostic methods.

The present solution comprises a system that employs software tools to extract skeletal body joint data from the video but integrates specialized data analytics to derive detailed gait features from the data. This rich dataset includes macro and micro gait features that provide insights into the gait dynamics of individuals. Extracted gait features are used to train machine learning models to identify subtle differences in gait that correlate with cognitive impairments. The trained machine learning models offer high accuracy and the capability to detect early signs of disorders, making them invaluable for early intervention strategies.

The system assesses the individual's gait during complex walking tasks (e.g., curve walking and walking in a figure-of-eight pattern) while adding cognitive load (e.g., counting backward). The system examines how cognitive impairment affects gait with and without cognitive load and also analyzes the cost of transitioning from no cognitive load to cognitive load. By evaluating these changes, the system detects subtle changes in gait patterns that are markers for early detection of cognitive impairment.

The present solution does not require the placement of markers on the body. By extracting a wide array of macro and micro gait features directly from video data, the present solution significantly expands the potential for detailed analysis without the obtrusive steps of attaching sensors or markers. Integrating descriptive statistical analysis and machine learning facilitates the identification of specific gait patterns indicative of AD and/or other forms of cognitive impairment, thus facilitating a more objective and accurate assessment of cognitive status compared to other methods. In some scenarios, sensor data acquired by body-mounted markers may optionally be used to validate the results from the video analysis.

The present solution can be used in various applications. These applications include, but are not limited to, hospital applications, clinic applications, specialty care center applications, assisted living facility and nursing home applications, rehabilitation center applications, university and research organization applications, private home applications, pharmaceutical company applications, and/or insurance company applications. For example, a neurologist in a neurology clinic can utilize the present system to screen subjects for early signs of cognitive impairment, AD, and related disorders. The present solution can serve as a preliminary assessment tool that complements diagnostic methods. A medical professional in an assisted living facility or nursing home can use the system to monitor residents regularly. The system allows for early detection of cognitive decline, enabling timely interventions that can enhance care quality and potentially slow the progression of symptoms. At rehabilitation centers for high-risk people, the system can be used by a physical therapist and/or rehabilitation specialist to provide ongoing assessments to track progress or detect setbacks. Research scientists and academics at universities and research organizations focused on geriatric medicine, neurology, and cognitive sciences can use the system for gait and cognitive health studies, enhancing research quality and providing robust tools for longitudinal studies. The system's ease of use and non-invasive nature can also be adapted for home use caregivers, allowing individuals or caregivers to conduct regular checks and share results with healthcare providers remotely. The system can be used by pharmaceutical companies involved in developing and testing medications aimed at treating or managing cognitive impairments and neurodegenerative diseases like Alzheimer's. The present system can be utilized by insurance companies to assess risk and manage claims related to cognitive impairment and neurodegenerative diseases, providing a basis for tailored insurance products.

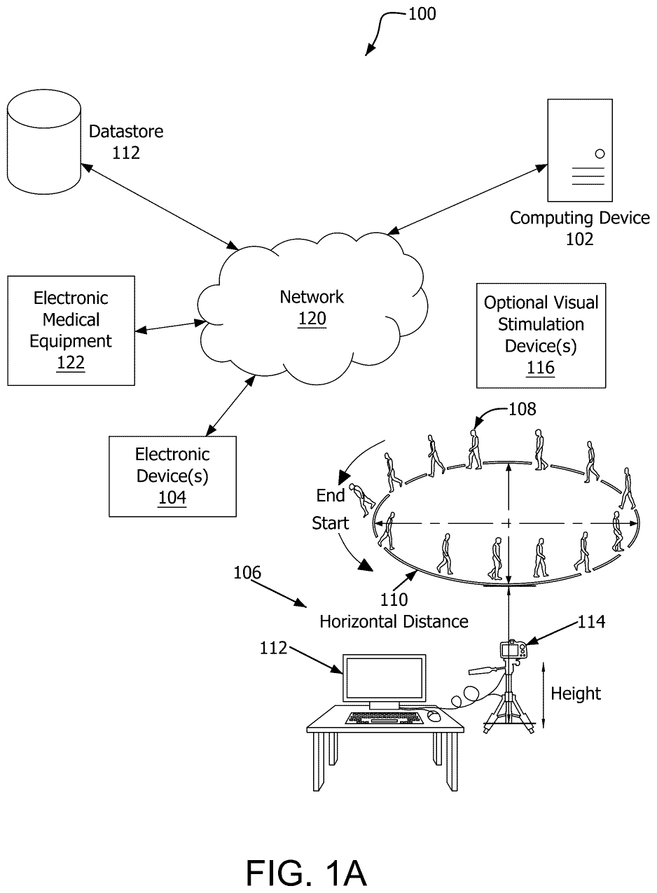

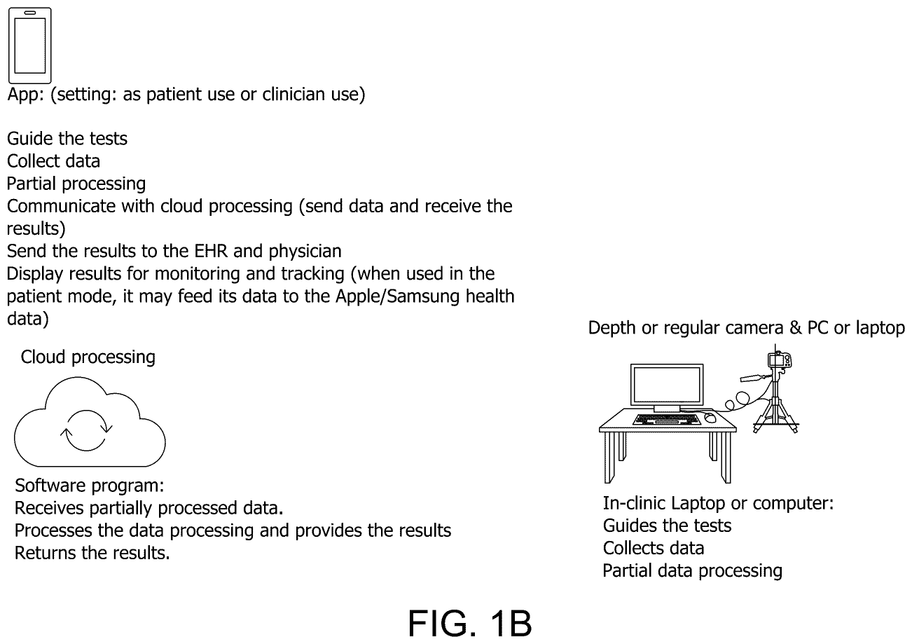

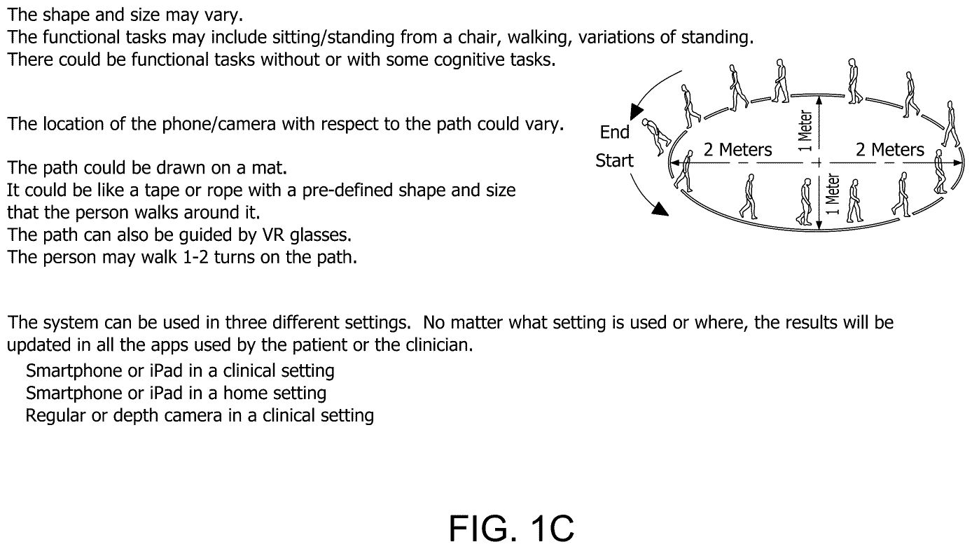

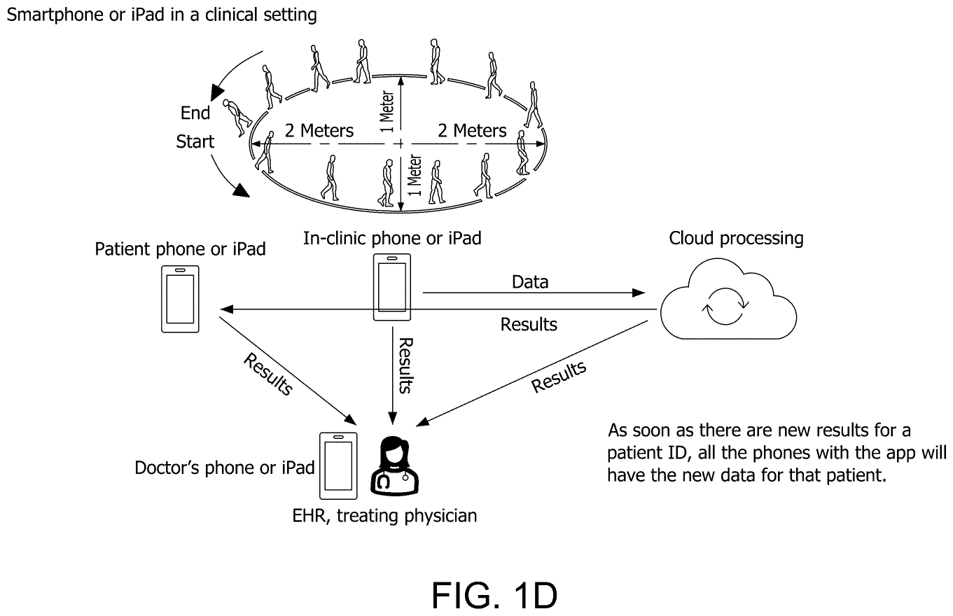

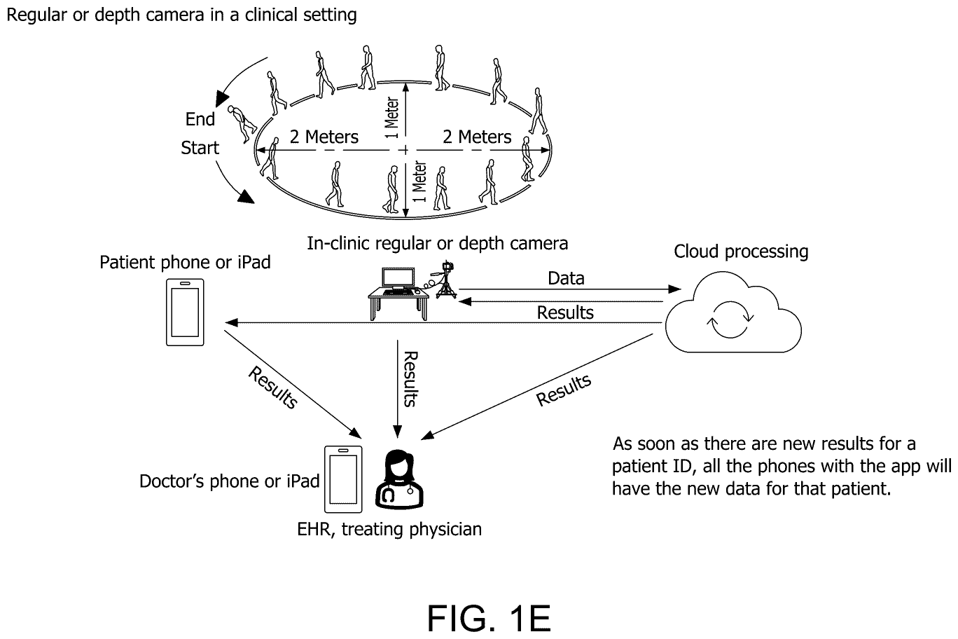

FIG. 1 provides an illustration of a system 100 implementing the present solution. System 100 comprises a computing device 102 configured to communicate with an electronic device(s) 104 and/or image sensor system 106 via network 120 (e.g., the Internet or Intranet). Computing device 102 can include, but is not limited to, a server, a medical professional's computer, a subject's computer, or cloud-based processing system. Electronic device(s) 104 is(are) configured to collect movement data and/or physiological or biophysical data of subject 108. The movement data and/or physiological or biophysical data can include data generated by one or more sensors of an electric device proximate (e.g., <20 feet from) to the subject 108 and/or an electronic device possessed by, worn by or coupled to the subject. Physiological or biophysical data can include, but is not limited to, height, weight, body dimensions, and/or body composition. Electronic device 104 can include, but is not limited to, a smart phone, an iPad, a wearable device, and/or a sensing device. The wearable device may include a smartwatch, a virtual reality (VR) device (e.g., smart glasses), a wearable patch or sensor, a fitness tracker, and/or a health management device. The sensor(s) and/or sensing device can include, but is(are) not limited to, an electrocardiogram (ECG) device, an electromyogram (EMG) device, an electroencephalogram (EEG)-based neural sensor, an optical sensor, an ultrasonic sensor, a imaging device, a camera, a location sensor (such as a global positioning system (GPS) sensor), an electrical sensor, a microphone, a vibration sensor, an accelerometer, a gyroscope, a pressure sensor, a pressure-sensitive walkway, and/or a wearable inertial measurement unit. The camera can include a depth camera, a (near-) infrared (IR) camera, a 3D camera, and/or a 360° camera.

Electronic device 104 may be worn by or otherwise coupled to a subject 108 of cognitive testing. The cognitive testing is performed to determine whether subject 108 shows signs of a problem with memory, thinking or other brain functions. During the cognitive testing, the subject 108 travels along a path 110. While the path is being traversed, movement data may be generated by a vibration sensor and/or accelerometer of electronic device 104 (e.g., a smart watch, etc.). The physiological or biophysical data may be generated by a heart rate sensor, a temperature sensor, a breathing rate sensor, a pupil dilation sensor, a moisture sensor, and/or an eye movement sensor of a wearable device, a smart phone, and/or sensor(s) mounted to a wall, desk or other structural object in proximity to the path.

The cognitive testing is facilitated by image sensor system 106. Image sensor system 106 is configured to capture video of subject 108 walking along path 110. Image sensor system 106 can include, but is not limited to, an optional computing device 112 and a camera 114. Computing device 112 can include, but is not limited to, a personal computer, a desktop computer, or a portable computer. Camera 114 can include, but is not limited to, a mobile device camera), a video camera, a depth camera, and/or any other device configured to capture videos of an environment. Other

While walking along path 110, subject 108 may be instructed to perform an action that increases or decreases his(her) cognitive load. Cognitive load is the amount of mental effort required to process information. For example, subject 108 may be instructed to count forwards and/or backwards as (s)he travels along path 110. Additionally or alternatively, subject 108 may be instructed to focus on one or more images in the vicinity or output by a mobile device, read text, recite the alphabet forwards and/or backwards, recite and/or repeat a particular phrase, solve a word problem, solve a mathematical problem, and/or complete a puzzle. The present solution is not limited to the listed actions for increases or decreases the cognitive load of the subject.

Additionally or alternatively, system 100 may comprise visual stimulation device(s) 116 to change the cognitive load. Visual stimulation device(s) 116 are configured to provide a visual stimulus to the subject. The visual stimulus can include, but is not limited to, interactive light patterns that may be counted or identified. The visual stimulus may be non-repetitive light pattern, a repetitive light pattern, a randomized sequence of colored lights or light patterns, and/or a defined sequence of colored lights or light patterns. As such, visual stimulation device(s) 116 can include, but is not limited to, lights and/or light emitting diodes (LEDs). The lights or LEDs may be integrated along path 110. The complexity and speed of each light pattern may vary, making the system adaptable to users with different cognitive abilities and providing a tailored assessment tool that can be scaled according to the user's cognitive status. Thus, characteristics of the visual stimulus may be based on the subject's current or known cognitive status. The characteristics of visual stimulus may be tailored to, for example, the subject's age, gender, medical evaluation(s) or test scores, and/or standardized test scores.

Path 110 is shown as being an elliptical path. The present solution is not limited to circular paths. Path 110 may alternatively comprise, for example, an oval path, a circular path, a rectangular path, a curvy path, a figure-of-eight path, and/or a straight-line path. Path 110 may be painted on a floor, marked on mat disposed on top of the floor, and/or otherwise marked on the floor (e.g., by tape, and/or via a virtual reality overlay on a real-world environment, for example, via VR glasses). Path 110 has a horizontal distance DH selected in accordance with a given application. DH can be any number greater than zero. For example, path 110 has a horizontal distance of two meters. One or more pieces of furniture may be disposed at the start of path 110, along path 110 and/or at the end of path 110 for providing additional information that is collected for cognitive assessment. For example, the piece of furniture may include, but is not limited to, a chair from which a sitting subject stands and/or on which a standing subject sits.

Path 110 may be painted or otherwise marked on a floor of a building. Additionally or alternatively, all or a portion of path 110 may be projected onto the floor by electronic device(s) 102, 112. Virtual markers and/or landmarks may additionally or alternatively be projected into the floor by electronic device(s) 102, 112.

The captured video and/or image(s) is(are) processed by device(s) 102, 104 and/or 114 to assess the balance of subject 108, assign a score to the subject's balance based on the assessment, assess a gait of the subject 108, assign a score to the subject's gait based on the assessment, measure an efficiency of the subject's walking, assess a risk of the subject falling, detect early signs of cognitive impairment of the subject 108 based on a gait and/or balance pattern, and/or diagnose AD or other disorder based on the gait and/or balance pattern. The subject's balance may be evaluated in terms of the following balance features—posture, unsteadiness, difficulty walking, time to stand from a sitting position, time to sit from a standing position, anterior-posterior (AP) velocities, center of mass medial-lateral (ML) displacement, and/or statistical metrics. The statistical metrics can include, but are not limited to, averages, standard deviation and/or other statistical values. The subject's gait may be evaluated in terms of the following gait features—step length, a step count, a step velocity, a stride length, a stride count, a stride velocity, an average velocity, a cadence, a cycle time, a walking duration, foot positions, joint angles, and/or statistical metrics. The statistical metrics can include, but are not limited to, standard deviation, mean, median, left-to-right ratio, symmetry, asymmetry, and/or variability coefficient. The gait features may be obtained by: extracting skeletal body joint data from the video and/or image(s); and performing data analytics using the extracted skeletal body joint data to obtain macro and/or micro gait features providing insight into gait dynamical of subject 108. The macro gait features can include, but are not limited to, a walking duration, an average velocity, a step count, and a stride count. The micro gait features can include, but are not limited to, micro spatial features and/or micro spatiotemporal features (e.g., step velocity, stride velocity, foot position change during a gait subphase), and/or at least one gait cycle subphase duration.

The captured video by image sensor 106 may be stored in local memory of the device(s) 102, 106, memory of computing device 102, and/or in a remote datastore 112. Datastore 112 can include, but is not limited to, a database. Other information may also be stored in datastore 112. The other information can include, but is not limited to, movement data, physiological or biophysical data, extracted skeletal body joint data, results of a cognitive assessment or test, gait features, balance features, and/or training data for one or more machine learning models. The training data can include, but is not limited to, the extracted skeletal body joint data, gait features and/or balance features.

The gait features can be used to train machine learning models to (i) identify subtle difference(s) in the subject's gait from one or more first reference gaits and/or (ii) correlate the identified difference(s) to one or more types of cognitive impairment. The first reference gaits can include a gait of the subject with no cognitive load or a normal or expected gait of another person without any cognitive impairment. The gait features can be used to train machine learning models to (iii) identify similarities in the subject's gait with one or more second reference gaits and/or (iv) correlate the identified similarities with one or more types of cognitive impairment. The second reference gaits can include a gait of the subject with a given cognitive load, an abnormal or unexpected gait of another person without any cognitive impairment, and/or a gait of another person with known cognitive impairment.

In the case of the depth camera, a web application on a computing device (e.g., a laptop) may be used to collect video data and process the data. The web app may be configured to send the results to EHR, treating physician. Also, there may be an app on the subject's personal device (e.g., phone) that shows the results and also has the ability to display and track the results over time.

In the case of a smartphone or iPad camera, there may be an app on the device that is used for collecting data. The app may be configured to perform some of the data processing and then send the processed data to a cloud or other computing device to continue the process. So, there may be another coding on the cloud or the computing device to receive this data. Once the data is processed, the results may be sent to electronic health record (her), the treating physician, the subject's phone app, etc.

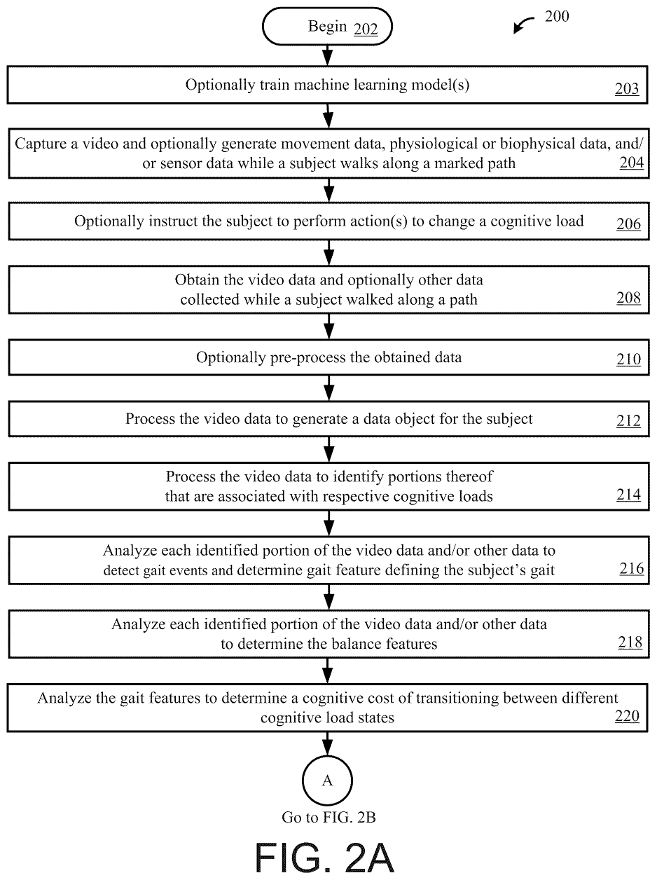

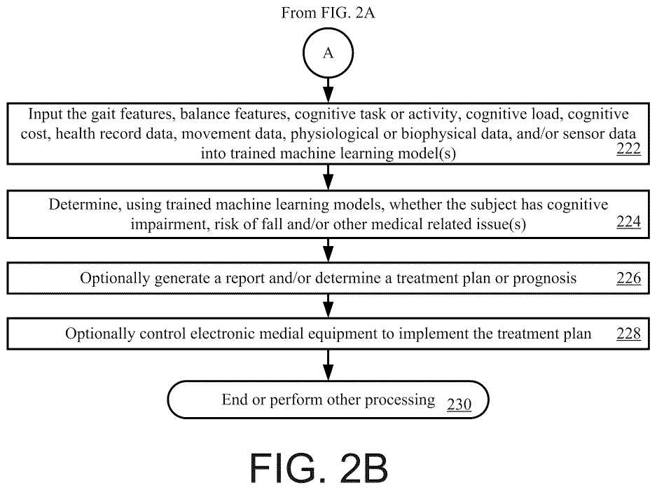

FIG. 2 provides a flow diagram of a method 200 for diagnosing a subject with a cognitive impairment or disorder. Method 200 may be performed by system 100 of FIG. 1 and/or device(s) 102, 104, 112 thereof. In some scenarios, method 200 may be at least partially implemented by a computing device (e.g., computing device 102 and/or 112 of FIG. 1) and/or implemented as an application executing on a mobile device such as a smart phone. The operations of method 200 may be performed in the same or different order than that shown.

Method 200 begins at block 202 and continues to optional block 203 where one or more machine learning models are trained. The machine learning model may be configured to continuously learn from subject and/or subject data. Machine learning models may be trained by, for example: obtaining gait feature and balance features for a plurality of individuals with and without cognitive impairments; and using the gait features and balance features to train machine learning models to detect patterns or behaviors indicating the presence of cognitive impairment and/or the lack of presence of cognitive impairment. Patterns in gait features and/or balance features may be correlated to one or more types of cognitive impairment or disorders. Movement data, physiological data and/or biophysical data can also be used by a machine learning models to learn patterns or behaviors of cognitive impairment or disorders. Any machine learning models can be used herein without limitation. For example, one or more of the following machine learning models are employed here: supervised learning; unsupervised learning; semi-supervised learning; and reinforcement learning. In a supervised learning model, the model learns a function that maps an input (also known as feature or features) to an output (also known as target) during training with a labeled data set (or dataset). In an unsupervised learning model, the algorithm discovers patterns among data. In a semi-supervised model, the model learns a function that maps an input (also known as feature or features) to an output (also known as a target) during training with both labeled and unlabeled data. Such machine learning models can include, but are not limited to, a support vector machine (SVM) classifier, a deep learning model, and/or a statistical analyzer. These patterns or behaviors can then be used to dynamically diagnose cognitive impairment or disorders. The gait and balance features may be obtained by performing the same or similar operations of block 204-220 for one or more individuals with and/or without cognitive impairment or disorder. These operations of block 204-220 will be discussed below.

Additionally or alternatively, the machine learning model(s) may be trained by identifying one or more significant gait features or balance features between at least one AD data set and at least one HC data set and adjusting the one or more significant gait features or balance features using an analysis of covariance (ANCOVA) technique. Such data including the identified significant gait feature(s) or balance feature(s) can be used to train the machine learning model(s). Adjusting the significant gait feature(s) or balance feature(s) can include adjusting for age, weight, and/or education for each subject in the AD data set(s) and/or the HC data set(s). Such data (e.g., a subject's age, weight, education, etc.) can be retrieved from a datastore (e.g. datastore 112 of FIG. 1) storing a subject's EHR.

Next, method 200 continues with cognitive testing of a subject. The cognitive testing begins with block 204 where a video is captured by a camera (e.g., camera 104 and/or 114 of FIG. 1) while a subject (e.g., subject 108 of FIG. 1) walks along a marked path (e.g., path 110 of FIG. 1). Other data may also optionally be generated while the path is being traversed. The other data can include, but is not limited to, movement data, physiological or biophysical data, and/or sensor data.

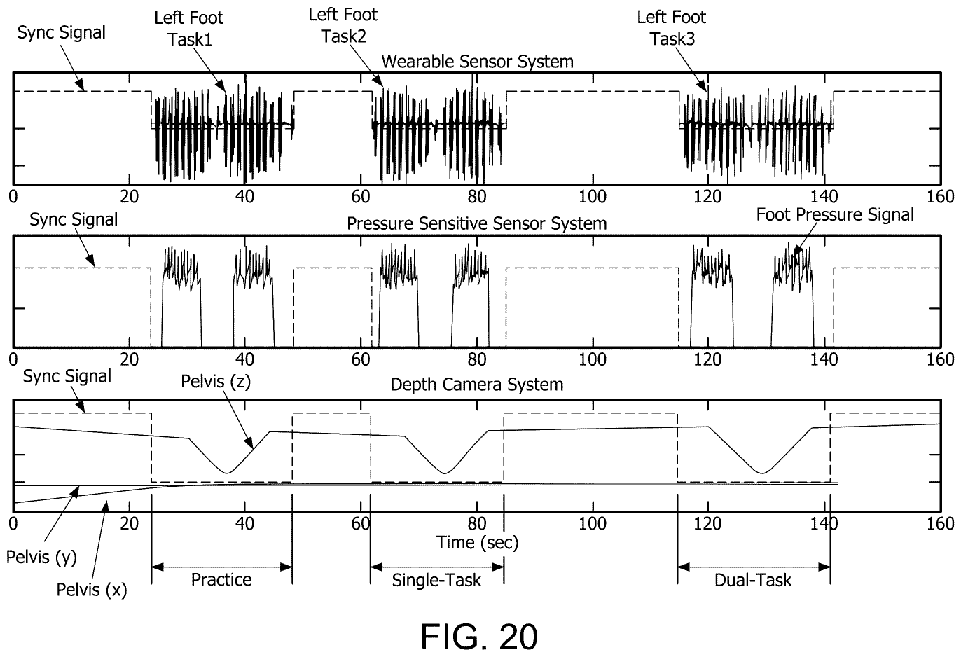

In some scenarios, the other data is acquired in 204 by sensors 104 worn by the subject and/or are being walked on by the subject. For example, the sensors 104 may comprise inertial measurement units that are secured to the subject by straps and placed on the dorsal surface of the left and right feet of the subject, as well as the lower back at the level of the fifth lumbar vertebra. The sensors 104 may additionally or alternatively comprise a mat with integrated pressure sensors. The pressure sensitive mat is configured to (1) identify heal strikes and toe-off events, and (2) compute spatiotemporal gait parameters (such as step length, stride length, cadence and gait speed). Thus, in this example, data is collected or otherwise acquired by three sensor systems—the depth camera 114, the wearable inertial measurement units 104, and the pressure sensitive mat 104. Computing device 112 may act as a controller for the three sensor systems to ensure precise temporal alignment for data acquisition. In this regard, the computing device 112 may transmit or otherwise communicate trigger signals to the sensor systems at the beginning and end of each task for coordinating data acquisition thereby. The trigger signals cause synchronization of the depth camera data with the wearable sensor recordings. FIG. 20 provides graphs showing raw gait signals and synchronized trigger signals across the three sensor systems for a single subject, demonstrating how the signal transitions from a first level during non-task periods to a second level during active walking periods. For the pressure sensitive mat and depth camara systems which may record continuously, the triggers may serve as temporal markers to segment task-specific data. The wearable sensor systems align recordings with the active walking periods defined by the sync signal. The present solution is not limited to the particulars of this example.

The marked path may include an elliptical path, a circular path, a rectangular path, a curvy path, a figure-of-eight path, and/or a straight-line path. The movement data and/or physiological/biophysical data can include data generated by one or more sensors in proximity to the subject while (s)he walks the path and/or sensor(s) possessed by, worn by or coupled to the subject prior to, during and/or after (s)he walks the path. For example, the movement data may have been generated by a vibration sensor and/or accelerometer of a smart watch. The physiological/biophysical data may have been generated by a heart rate sensor, a temperature sensor, a breathing rate sensor, a pupil dilation sensor, a moisture sensor, and/or an eye movement sensor of a wearable device, a smart phone, and/or sensor(s) mounted to a wall, floor, ceiling, pillar, desk or other structural object in proximity to the path. The present solution is not limited to the particulars of this example.

As shown by block 206, the subject may optionally be instructed to perform one or more actions or tasks to increase and/or decrease his(her) cognitive load while traveling along the predetermined path. For example, the subject may be instructed to: recite the alphabet or count numbers in an incrementing or decrementing order; count lights of a light pattern; and/or identify objects or animals shown by light patterns. The present solution is not limited to the particulars of this example. Other types of cognitive load stimulus may be used here.

Next in block 208, a processor performs operations to obtain video data and optionally other data (e.g., movement data, physiological/biophysical data, and/or sensor data) collected while a subject (e.g., subject 108 of FIG. 1) walked along a path (e.g., path 110 of FIG. 1). These operations can include, but are not limited to, accessing a local memory or a remote datastore (e.g., datastore 112) and retrieving stored data therefrom.

Next in block 210, the processor performs operations to pre-process the obtained data. The pre-processing may include data format conversion, noise filtering, and/or data filtering for computation performance reasons. For example, the video data may be transformed from three-dimensional (3D) video data to two-dimensional (2D) video data, or transformed from 2D data to 3D data. It can be advantageous to transform data obtained in one form to another form in order to conserve or increase computing resources, as required in different contexts. For example, in order to improve or enhance collected data (e.g., where no high-resolution or depth camera is available), the processor may convert 2D data obtained via a mobile device to 3D data before proceeding with additional processing. Conversely, in order to conserve resources, the processor may convert 3D data obtained via a depth camera to 2D data prior to performing additional processing operations. The present solution is not limited to the particulars of this example.

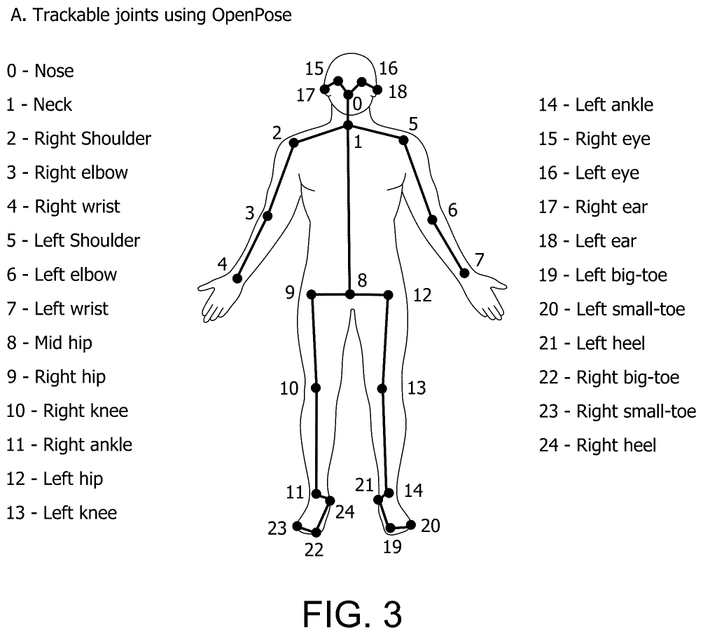

In block 212, the processor and/or trained machine learning model(s) perform(s) operations to generate a data object for the subject. These operations may involve: applying a 2D human pose estimation technique and/or other algorithm to detect and track the human body in the video; detect body joints within the video data; preprocessing or filtering signals from the detected body joints to remove noise signals; feeding them directly to machine learning models; and/or extracting relevant features corresponding with the detected joint(s). The 2D human pose estimation technique can include, but is not limited to, an OpenPose algorithm, and/or any other algorithm configured to estimate a 2D human pose. FIG. 3 provides an illustration showing possible joints 0-24 that may be detected in block 212.

In block 214, the processor and/or trained machine learning model(s) perform(s) operations to identify portions thereof that are associated with respective amounts of cognitive loads. For example, a first portion of the video data is identified as being associated with no cognitive load. A second portion of the video data is identified as being associated with a relatively small cognitive. A third portion of the video data is identified as being associated with a relatively large cognitive. A cognitive load type may be assigned to each of the three identified portions of the video data in block 214.

In block 216, each identified portion of the video data and/or other data is analyzed to detect gait events and determine gait features defining the subject's gait. This analysis may be conducted across a range of gait markers categorized into macro and micro-level parameters. Macro-level gait markers capture overall performance and include average gait velocity and cadence. In this regard, macro-level gate markers can include, but are not limited to, average velocity and cadence. Micro-level gait markers reflect more detailed aspects of the gait cycle. Micro-level markers may be further classified into the following: temporal markers (e.g., stride time, step time, stance time, swing time, single and double support time), spatial markers (e.g., stride length, step length), and spatiotemporal markers (e.g., stride velocity). These categories allow the system to measure both high-level and fine-grained gait characteristics under both single-task and dual-task walking conditions.

In some scenarios, the operations of block 216 involve analyzing a first portion of video data to detect gait events and determine a first set of gait features defining the subject's gait while having no cognitive load. The second portion of video data is analyzed to detect gait events and determine a second set of gait features defining the subject gait while having a relatively small cognitive load. The third portion of the video data is analyzed to detect gait events and determine a third set of gait features defining the subject gait while having a relatively large cognitive load. Each of the subject's gaits may be defined by gait features such as step length, a step count, a step velocity, a stride length, a stride count, a stride velocity, an average velocity, a cadence, a cycle time, a walking duration, foot positions, joint angles, and/or statistical metrics. The statistical metrics can include, but are not limited to, standard deviation, mean, median, left-to-right ratio, symmetry, asymmetry, and/or variability coefficient. The gait features may be timestamped with the time associated with the corresponding video data. The pressure sensitive mat data may be analyzed to detect heel strikes, detect toe-off events, and/or compute or otherwise determine spatiotemporal gate parameters. The spatiotemporal gate parameters can include, but are not limited to, step length, stride length, cadence and gait speed. The spatiotemporal gate parameters may be timestamped with the time associated with the corresponding pressure sensor data. The present solution is not limited to the particulars of this example.

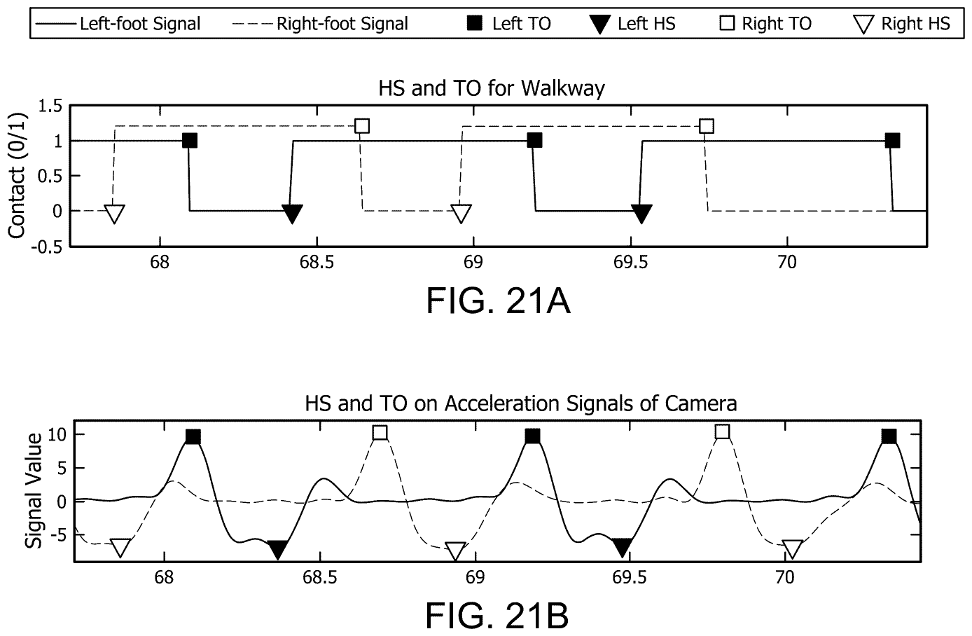

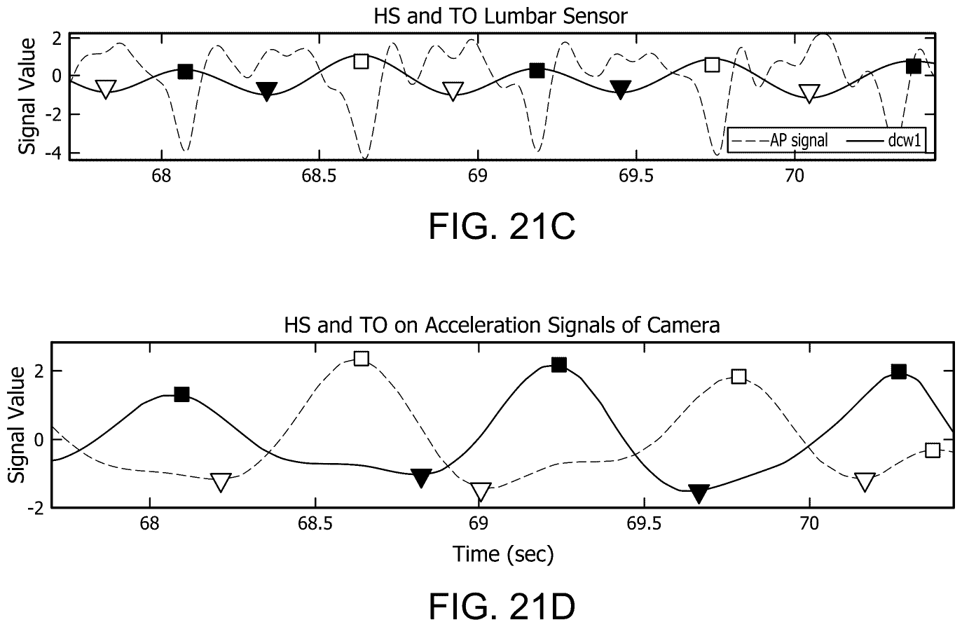

The wearable sensor data and/or pressure sensitive mat data may optionally be analyzed in block 216 to detect gait events and calculate temporal and spatial gait markers based on the detected events. These calculated temporal and spatial gait markers may be used to validate results from analyzing the video data. FIG. 21A provides a graph showing heel strike and toe-off events on the foot contact data for a representative pressure sensitive mat. FIG. 21B provides a graph showing heel strike and toe-off events for foot sensors. FIG. 21C provides a graph showing heel strike and toe-off events for a lumbar sensor. FIG. 21D provides a graph showing heel strike and toe-off events for an acceleration sensor.

Heel strike events and toe-off events may be detected, for example, using medial-lateral angular velocity from the foot-mounted inertial measurement units. A 12th-order, high-pass Butterworth filter, implemented in second-order sections, may be applied to remove low-frequency drift. FIG. 21B illustrates the detected heel strike events and toe-off events for the left and right foot sensors for the representative subject.

A gait event detection approach may be performed using the lumbar-mounted inertial measurement unit. The anterior-posterior acceleration signal (Z-axis of the accelerometer) is first preprocessed by linear detrending and low-pass filtering at 10 Hz using a second-order Butterworth filter. The filtered signal may then be integrated using the cumtrapz function and subsequently differentiated using the continuous wavelet transform (CWT). A Gaussian first-order (gaus1) wavelet may be applied at an estimated optimal scale. Local minima and maxima of the first-order differentiated signal may be identified as heel strike events and toe-off events, respectively. Spatiotemporal gait parameters may then be calculated from the timing of heel strike event and toe-off events, along with step length estimations based on an inverted pendulum model. FIG. 21D shows detected heel strike events and toe-off events for the left and right foot estimated using the lumbar-mounted inertial measurement unit.

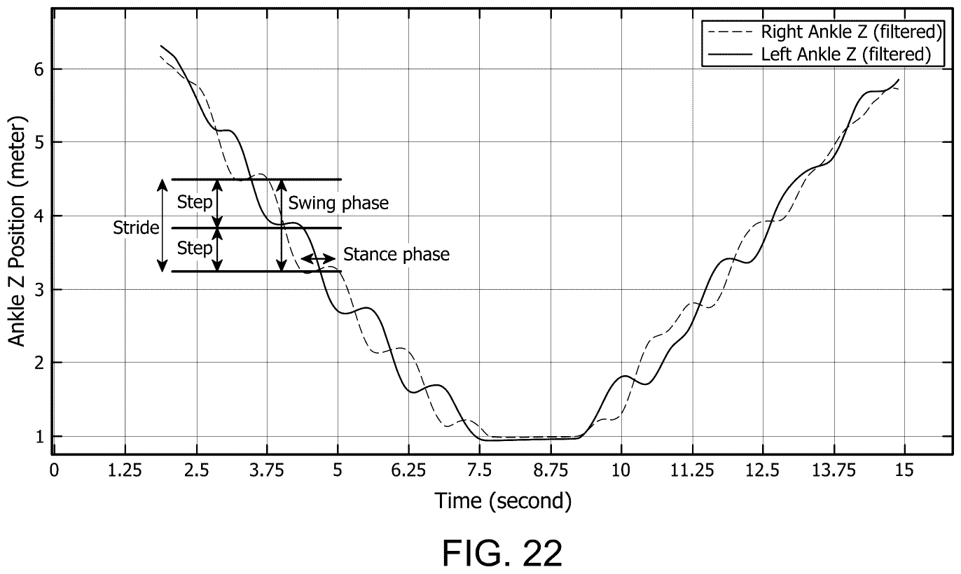

A gait event detection approach may be performed using joint movement signals output from the ankle mounted sensor(s). A sixth-order Butterworth filter with a 3 Hz cutoff frequency may be applied to joint movement signals to eliminate noise. To detect the gait cycles and their related gait markers (such as stance and swing phases, step and stride times, and length), the heel strike and toe-off occurrence is detected. The second gradient may be applied on the signals of the right and left ankles in the y-direction to convert the location signals of the ankle and foot to acceleration signals. The local maximum and minimum of the ankle acceleration signals may be identified as heel strike incidents and toe-off incidents, respectively (see FIG. 22). Next, various gait markers may be calculated using heel strike and toe-off incidents.

Temporal and spatial gait parameters may be calculated following toe-off and heel strike event detection. The temporal gait markers (e.g., step time, stride time, stance time, etc.) may be computed based on the timing of heel strike and toe-off events and are consistent across all sensing technologies. Formulas are presented below for the right foot, assuming the left foot initiates the gait cycle. Analogous formulas may be applied to the left foot by adjusting the event sequence accordingly. In contrast, the spatial gait markers (e.g., step length, stride length) may be derived using technology-specific methods that do not rely directly on heel strike and toe-off timing, and are therefore shown separately for each sensing technology.

| | Gait Marker | Formula |
|---|---|---|
| | Temporal Gait Markers | |
| Applied to all | Step Time | HS_{g} (i) - HS_{L} (i) |
| Technologies | Stride Time | HS_{g} (i + 1) - HSg (i) |
| | Stance Time | TO_{g} (i) - HS_{g} (i) |
| | Swing Time | Stride Time - Stance Time |
| | Single Support Time | HS_{g} (i + 1) - TO_{L} (i) |
| | Initial Double Support | TO_{L} (i) - HS_{R} (i) |
| | Time (DST) | TOg (i) - HS_{L} (i + L) |
| | Terminal Double | |
| | Support Time | |
| | (DST) | Initial DST + Terminal DST |
| | Double Support Time | |
| | Special Gait Markers | |
| Foot Sensor | Step Length | $SL = {K}^{4} \sqrt{{}^{a} max - {}^{a} min} ,$ |
| | | where aₘₐₓ and aₘᵢₙ are the |
| | | maximum and minimum vertical |
| | | acceleration values during one step. |
| | Stride Length | $Stride = {K}^{4} \sqrt{{}^{a} max - {}^{a} min} ,$ |
| | | applied over one stride. |
| Lumbar | Step Length | SL = 2\sqrt{2lh - h²} |
| Sensor | | (inverted pendulum model), where |
| | | 1 is foot length and h is vertical |
| | Stride Length | displacement during the step. |
| | | Stride = Step_{g} + Step_{L} |
| Azure Kinect | Step Length | [z (HS_{g} (i)) - z (HS_{L} (i))] |
| | Stride Length | [z (HS_{g} (i) + 1)) - z (HS_{g} (i))] |

where HS represents heel strike, TO represents toe-off, R represents right foot, L represents left foot, i represents current event, and i+1 represents next event.

Step length from the foot-mounted sensors was estimated using an empirical equation. The calculation requires determining the vertical acceleration component (aZ), derived from the foot's local acceleration signals ax and az using the relation a_{Z}=ax sin θ+a_{z} cos θ−g, where θ is the pitch angle estimated from gyroscope data, and g is the gravitational acceleration. The parameters aₘₐₓ and aₘᵢₙ represent the maximum and minimum values of a_{Z} within a single step, and K is a calibration constant used for subject-specific adjustment. Stride length may be calculated using the same formula, but aₘₐₓ and aₘᵢₙ are extracted over the duration of a full stride instead of a single step.

Step length from the lumbar-mounted sensor may be estimated using an inverted pendulum model. In this model, h denotes the vertical displacement of the center of mass, obtained through double integration of the vertical acceleration (aₓ) using the cumulative trapezoidal method (cumtrapz). The parameter l represents the pendulum length, which was approximated by the subject's foot length.

Step length from the depth camera may be calculated by subtracting the z-direction position of one foot at its heel strike from the z-direction position of the opposite foot at the time of its toe-off. Also, to calculate the stride length, the location of the same foot during two successive heel strikes is subtracted from each other.

In block 218, each identified portion of the video data and/or optional other data is analyzed to determine balance features. The operations of block 218 may be performed prior to, subsequent to, or simultaneously with the operations of block 216. For example, the first portion of video data is analyzed to determine a first set of balance features for the subject while having no cognitive load. The second portion of video data is analyzed to determine a second set of balance features for the subject while having a relatively small cognitive load. The third portion of the video data is analyzed to determine a third set of balance features for the subject while having a relatively large cognitive load. Each set of balance features can include posture(s), unsteadiness indicator(s), and/or walking difficulty indicator(s). Pressure sensor data and/or wearable sensor data may also be analyzed to determine balance features that may be used to validate the balance features determined based on the video data. The balance features may be timestamped with the time associated with the corresponding video data, pressure sensor data, and/or wearable sensor data. The present solution is not limited to the particulars of this example.

In block 220, the processor and/or trained machine learning model(s) perform(s) operations to analyze the gait features and/or balance features to determine a cognitive cost of transitioning between different cognitive load states. The cognitive load states can include, but are not limited to, a no cognitive load state, a small cognitive load state, and a large cognitive load state. These operations may involve: obtaining the gait features and/or balance features associated with the first portion of the video data; obtaining gait features and/or balance features associated with another portion of the video data; comparing the gait features associated with the first portion to the gait features associated with the another portion; comparing the balance features associated with the first portion to the balance features associated with the another portion; and determining a cognitive cost C of transitioning from a first cognitive load state to a different second cognitive load state based on results of the comparing operations. The cognitive cost may be determined, for example, by computing a weighted combination of the differences between gait features. The cognitive cost may be defined by the following mathematical equation (1).

$$C = {w}_{1} {d}_{1} + {w}_{2} {d}_{2} + … + {w}_{n} {d}_{n} \qquad ( 1 )$$

where w₁, w₂, . . . , wₙ are weights, d₁ represents a different between a first feature (gait or balance) (e.g., step length) in the two portions of video data, d₂ represents a different between a second feature (gait or balance) (e.g., step count) in the two portions of video data, and dₙ represents a different between an N^{th} feature (gait or balance) (e.g., stride velocity) in the two portions of video data. The cognitive cost C may indicate whether the subject has a cognitive impairment. For example, a low cognitive cost may indicate that the subject is unlikely to have a cognitive impairment, while a high cognitive cost may indicate that the subject is likely to have a cognitive impairment. A person with a cognitive impairment may experience more difficulties in completing a movement task (e.g., walking, standing) while simultaneously performing a cognitive task. Accordingly, such data can be used to enrich/improve the output of the analysis and facilitate a more comprehensive evaluation of gait and balance related features.

In block 222, the gait features, balance features, cognitive task or activity, cognitive load, cognitive cost, health record data, movement data, physiological data, and/or biophysical data are input into trained machined learning model(s). The health record data can include, but is not limited to, an age, a weight, an education, medication(s), other demographic information, family medical history, and/or previous diagnosis of a disease or other heath issue.

The inputs may be used in block 224 to determine whether the subject has cognitive impairment or disorder, risk of fall and/or other medical related issue(s). This determination can include, for example, detecting or predicting pre-clinical AD, detecting or predicting dementia, detecting or predicting sub-types of dementia, detecting or predicting a severity of cognitive impairment or AD, quantifying how cognitive impairment or disorder is progressing, and/or indicating an underlying etiology of the cognitive impairment. The machine learning models may be trained to detect that the subject has cognitive impairment or disorder, risk of fall and/or other medical related issue(s) based on patterns the subject's movement while walking, sitting, transitioning from sitting to standing (or vice versa), and/or performing other functional activities. For example, in some scenarios, the input may be processed or analyzed by the machine learning model(s) to: (i) identify subtle difference(s) in the subject's gait or behavior from one or more first reference gaits or behaviors; (ii) correlate the identified difference(s) to one or more types of cognitive impairment or disorder; (iii) identify similarities in the subject's gait or behavior with one or more second reference gaits or behaviors; and/or (iv) correlate the identified similarities with one or more types of cognitive impairment. The first reference gaits or behaviors can include a gait or behavior of the subject with no cognitive load or a normal or expected gait or behavior of another person without any cognitive impairment. The second reference gaits or behaviors can include a gait or behavior of the subject with a given cognitive load, an abnormal or unexpected gait or behavior of another person without any cognitive impairment, and/or a gait or behavior of another person with known cognitive impairment. The another person may have similar cognitive task or activity, cognitive load, health record data, physiological data, and/or biophysical data associated therewith.

Block 224 may additionally involve generating score(s) by the trained machine learning model(s) and/or statistical analysis of various information. The information can include, but is not limited to, outputs of the trained machine learning model(s). Based on healthy people's performance, a composite score may be derived from their normative data. This score can be used to detect people with cognitive impairment using a cutoff score. It can also be used for longitudinal monitoring of people's cognitive decline. Instead of detecting as 0 or 1, a score may range from 0 to 1, where higher values may indicate the disease progression. As the lower the score gets, it may show that the intervention is being effective.

In block 226, the processor may perform operations to generate a report and/or determine a treatment plan or prognosis for the subject based on the output of the machine learning model(s). The report can include, but is not limited to, predictions of cognitive impairment severity and/or progression, recommendations for improving and/or reducing progression of cognitive impairment, recommendations for actions and/or tasks to assist with slowing down cognitive impairment, and/or recommendations for physical therapy. The report may be integrated into the subject's EHR and/or used to monitor the progression of the subject's cognitive impairment over time. Block 226 may also involve: displaying the report on a display screen of a local device (e.g., device(s) 102, 112 of FIG. 1); and/or transmitting the report over a network (e.g., network 120 of FIG. 1) to a remote device (e.g., computing device 102 of FIG. 1). Treatment plans can vary depending on the disease state and relevant subject factors. The operations related to providing diagnosis, prognosis, treatment options, treatment plans and/or monitoring can be performed using one or more computing devices (e.g., computing device(s) 102, 104, 112 of FIG. 1).

The processor may additionally or alternatively perform operations in block 228 to control electronic medical equipment (e.g., electronic medical equipment 122 of FIG. 1) to implement the treatment plan. For example, a medical device may be controlled to increase blood flow to the subject's brain, parameters of a hearing aid may be reconfigured to reduce the rate of cognitive decline in the subject, parameters and/or settings of a cognitive orthotic system may be reconfigured to better assist with completing daily tasks by the subject, and/or parameters of medication dispensing devices may be reconfigured to increase or decrease the dose of medicine and/or the timing between medicine dispenses. Subsequently, method 200 continues to block 230 where it ends or other operations are performed (e.g., return to block 202, 203 or 204).

A system implementing method 200 may be employed in hospitals, clinics, specialty care centers (e.g., neurology clinics), and the like to screen subjects for early signs of cognitive impairment, AD, and related disorders. Such a system can serve as a preliminary assessment tool that complements or replaces other diagnostic methods. Assisted living facilities and nursing homes can use a system implementing method 200 to monitor residents regularly. Such a system allows for early detection of cognitive decline, enabling timely interventions that can enhance care quality and potentially slow the progression of symptoms. At rehabilitation centers for high-risk people, ongoing assessments can be provided to track progress or detect setbacks.

First Experimentation: Human Pose Estimation and Gait Analysis with Convolutional Neural Networks for AD Detection

AD is a progressive neurodegenerative condition characterized initially by memory issues, escalating into significant functional impairments in daily activities such as language, mobility, and driving, ultimately increasing the risk of death in older adults. Early detection of AD can significantly slow the disease's progression through timely medical intervention and aid in managing the challenges faced by affected individuals and their caregivers through rehabilitation programs and lifestyle adjustments.

The present study introduces a cutting-edge approach to gait and/or balance analysis that leverages a single regular camera combined with the open-source algorithm, a deep learning-based computer vision technique for human pose estimation (HPE). This novel method simplifies data collection while ensuring privacy and reducing costs, and notably, it does not require the placement of markers on the body. By using a wide array of macro and micro gait features, the present approach significantly expands the potential for detailed analysis without the obtrusive steps of attaching sensors or markers. Integrating descriptive statistical analysis and machine learning, the present solution aims to identify specific gait patterns indicative of AD, thus facilitating a more objective and accurate assessment of cognitive status compared to other methods.

Study Population: Participants underwent comprehensive checkups for assessment and diagnosis of cognitive impairments due to AD using brain imaging, blood tests, and MoCA neuropsychological test, as well as the geriatric depression Scale (GDS) for depression measurements. In total, seventy-three older adults without any cognitive impairment as the HC group and thirty-four AD subjects who could perform the physical tests independently and did not have gait deficits because of surgery like knee surgery and medicine affecting their gait were chosen and completed the gait tests.

Image-Based Gait Analysis: To assess gait for AD detection, participants walked a ten-meter curved path, validated and widely used in clinical settings. Participants initially undertook a single trial as a practice session before completing three trials for detailed analysis. Between each trial, individuals were allowed rest periods ranging from one to three minutes to ensure optimal performance during the tests.

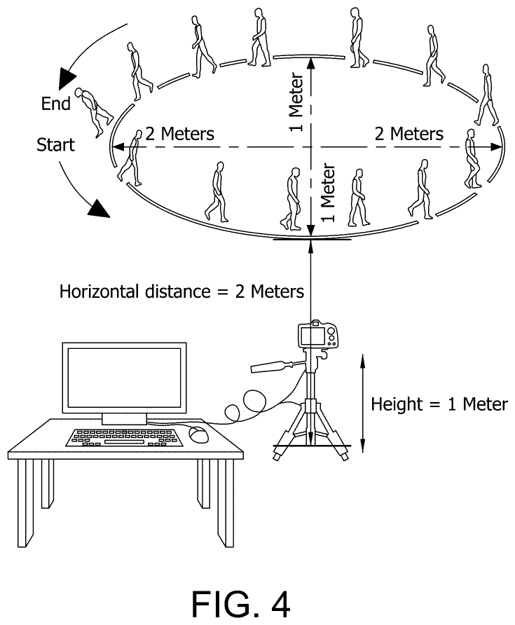

Data capturing of the gait tests was achieved using a high-definition single camera, set to a resolution of 1920×1080 pixels and operating at a frame rate of thirty frames per second. The camera was securely mounted on a tripod to maintain a stable lateral view of the participants' walking path. The data acquisition setup was managed using a computing device configured to facilitate efficient processing and storage of the collected gait data. The equipment arrangement and methodology for recording the ten-meter curved path walking tests are depicted in FIG. 4, illustrating the setup utilized for capturing the gait data of participants.

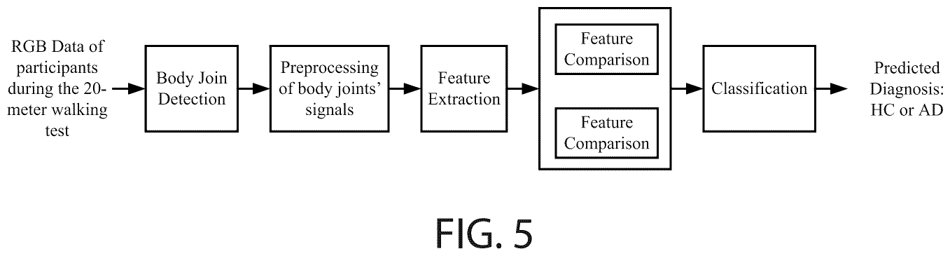

Innovations in Image Processing for AD Detection: After collecting RGB data from participants during their curved path walks, a series of processing steps were performed to facilitate AD detection from the recorded data. This approach involved several key phases: detecting body joints within the RGB images, preprocessing the signals from these detected joints, extracting relevant features, conducting descriptive statistical analysis for feature selection, and employing machine learning models to identify AD in older adults. FIG. 5 delineates these phases of the present algorithm.

Automated body joint detection: The automated detection of body joints from RGB images was achieved through the open-source algorithm. A real-time 2-D HPE technique is characterized by its bottom-up approach. Utilizing an advanced multi-stage convolutional neural networks (CNNs), the open-source algorithm identifies the locations of body joints and connects them, creating a skeletal structure through Part Affinity Fields. This method's ability to provide real-time pose estimations offers significant advantages, especially in scenarios involving multiple individuals, setting it apart from top-down estimation techniques. In the research, the Python implementation of the open-source algorithm was used for HPE to identify twenty-five body joints within the captured images, covering key anatomical landmarks such as the feet, elbows, and hands. FIG. 3 provides a visual representation of the joints identified and the skeletal framework constructed from RGB images obtained during the ten-meter curved path walking tests, highlighting the effectiveness of the open-source algorithm in the study.

Signal preprocessing and conversion: As participants navigated the curved path, the movement of their body joints produced signals in both horizontal (x) and vertical (y) directions over time, which are important for extracting key gait features such as average velocity and step length. To ensure the accuracy of these signals, a six-order Butterworth Filter was applied with a cut-off frequency of 3 Hz to remove any extraneous noise. These dimensions were converted into real-world measurements after the initial detection of body joint positions by the open-source algorithm and their representation in pixel-based measurements. This conversion to real dimensions facilitated subsequent analysis of the data.

Extraction and selection of gait features: From the signals derived from detected body joints during gait assessments, the system extracted a total of forty-eight gait features. These were categorized into six macro features (like walking duration, average velocity, and step and stride counts), twenty-four micro temporal features (such as stance and swing durations), twelve micro spatial features (including step length and its variability), and six micro spatiotemporal features (for example, step and stride velocity). Macro features calculation involved methods such as dividing the total displacement of the ankle joint by the test duration for average velocity. For micro features, identifying gait cycles and their phases was essential, accomplished by analyzing distance signals from ankles and using their peaks to segment gait into stance, swing, single support, and double support phases. Statistical analysis of these features then provided metrics of mean, median, and variability, the latter expressed as the standard deviation's percentage of the mean.

A systematic feature selection process was implemented to refine the feature set for AD detection, beginning with a descriptive statistical analysis to consider potential confounding demographic and clinical factors. Tests such as Shapiro-Wilk, unpaired t-tests or Mann-Whitney U tests, and Chi-square tests were applied to ensure that comparisons between the AD and HC groups accounted for variable distribution and category differences. Significant gait features that were distinguished between the groups were then adjusted for confounders like age, weight, and education level using ANCOVA. These adjusted features were then compared to pinpoint those with statistically significant differences. This selection was further refined through correlation analysis, where among correlated features (r>0.9), the one with the most significance (lowest p-value) was chosen as the prime indicator for distinguishing between AD and HC groups, streamlining the analysis to the most discriminative features.

Machine learning-based AD classification: For the classification of AD from the study's gait analysis data, an SVM classifier was used. SVM was selected due to its proven effectiveness in accurately categorizing distinct groups, an advantage especially relevant for analyses with limited data sizes. Its principal strength lies in transforming data into a higher dimension where linear separation is possible, facilitating the distinction between participants diagnosed with AD and those categorized as HC.

The reliability and generalizability of the classification model was ensured by adopting a 5-fold cross-validation strategy. This method divides the dataset into five parts, using four for training and the remaining one for testing, iteratively. For each training phase, the data was further segmented, allocating 80% for model training and 20% for validation. This approach helped fine-tune the model's performance, optimizing it for higher accuracy in subsequent testing phases. Key to the success of the SVM classifier is the careful selection of kernels and hyperparameters, tasks accomplished using grid search techniques. This systematic search allowed identification of the optimal settings for the SVM model, ensuring the best possible performance in classifying AD and HC participants based on their gait features. To uphold the integrity of the testing process and avoid the potential bias of feature selection leakage, the selected gait features for each classification task were determined solely from the training data. This precaution ensured that the validation and testing of the machine learning model were conducted on entirely unseen data, thus providing a more accurate and trustworthy assessment of its capability to classify AD from gait features.

Results: The following TABLE 1 shows study participants' demographic and clinical information. A comparison of this information using descriptive statistical analysis showed significant differences between AD and HC participants in terms of age, weight, and years of education, with no significant differences in gender, height, and GDS variables between the two study groups. Also, the comparison of the cognitive assessment using neuropsychological test scores of MMSE and MoCA revealed that older adults with AD had significantly lower cognitive scores than HC participants.

TABLE 1
Demographic and clinical information of study participants

| Characteristic | HC (N = 73) | AD (N = 34) | p-value |
|---|---|---|---|
| Age (years) | 64.67 ± 5.73 | 76.06 ± 7.24 | <0.001* |
| Female, N (%) | 38 (53) | 19 (56) | 0.715 |
| Height (cm) | 168.09 ± 6.25 | 165.17 ± 6.45 | 0.057 |
| Weight (kg) | 69.32 ± 9.44 | 6.09 ± 8.69 | 0.038* |
| Education (years) | 13.43 ± 2.38 | 10.88 ± 4.11 | 0.001* |
| MMSE | 28.96 ± 0.91 | 22.56 ± 3.86 | <0.001* |
| MoCA | 28.03 ± 0.05 | 20.76 ± 2.49 | <0.001* |
| GDS | 2.04 ± 1.24 | 3.29 ± 1.85 | 0.389 |

Mean ± Standard deviation was shown. N = Number of participants; HC = Healthy Cognitive Control Group; AD = Alzheimer's Disease; MMSE = Mini-Mental State Examination (maximum score, 30); MoCA = Montreal Cognitive Assessment (maximum score, 30); GDS = Geriatric De-pression Scale (maximum score, 15),
*shows the significant difference for the level of p < 0.05.

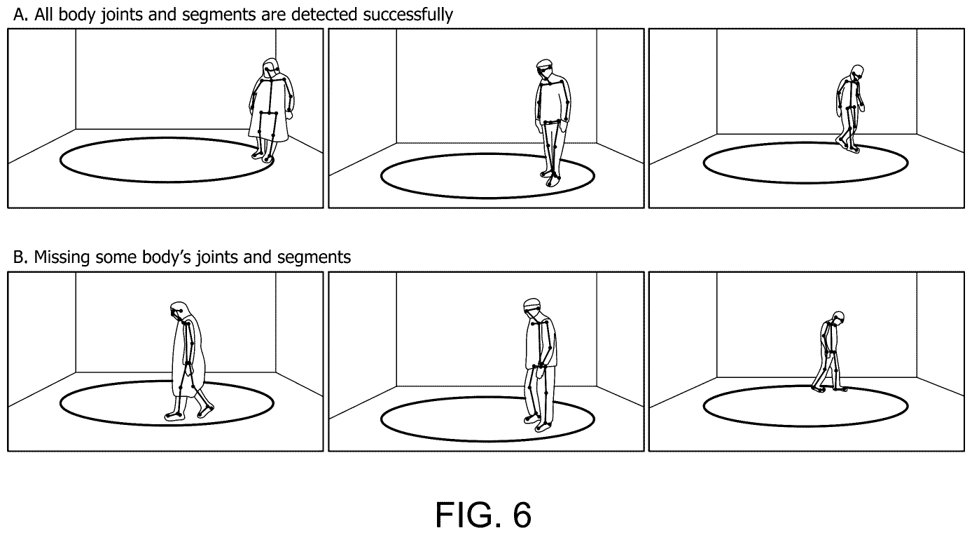

FIG. 6 illustrates the open-source algorithm's performance across different situations within the dataset. In a majority of the frames, the open-source algorithm effectively identified and tracked body joints and segments without any occlusion by external objects or self-occlusion, except for minor cases where partial occlusion of segments, such as the knees, occurred as depicted in top portion of FIG. 6A. However, instances of full occlusion due to overlapping body parts significantly impacted the open-source algorithm's ability to detect and track certain joints, particularly in the upper body wrist, elbow, and shoulder joints and the segments connecting these joints faced detection issues in frames where one side was entirely obscured as illustrated in bottom portion of FIG. 6B. This variability in detection underscores the importance of acknowledging the open-source algorithm's limitations under specific conditions, particularly when analyzing gait data for movement assessment depending on the application in hand.

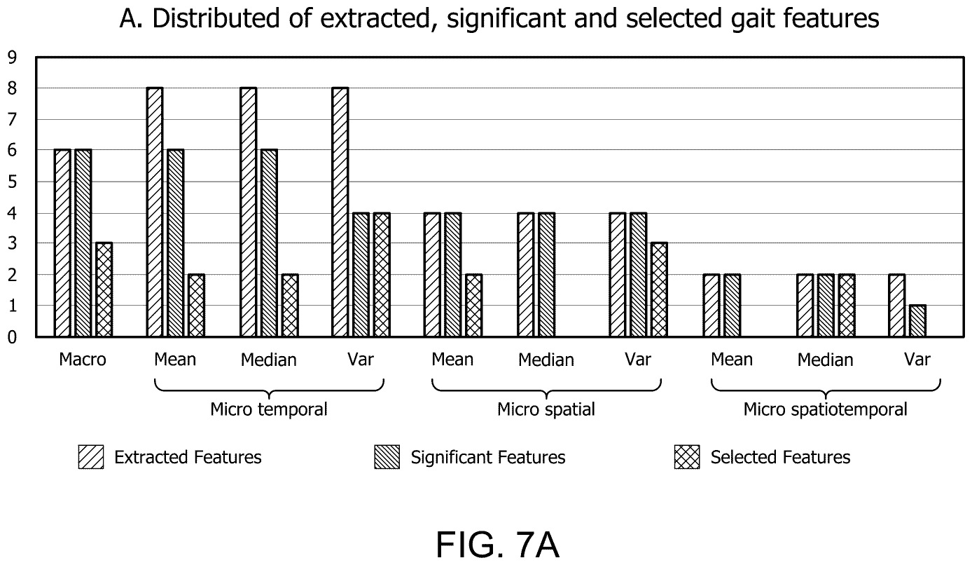

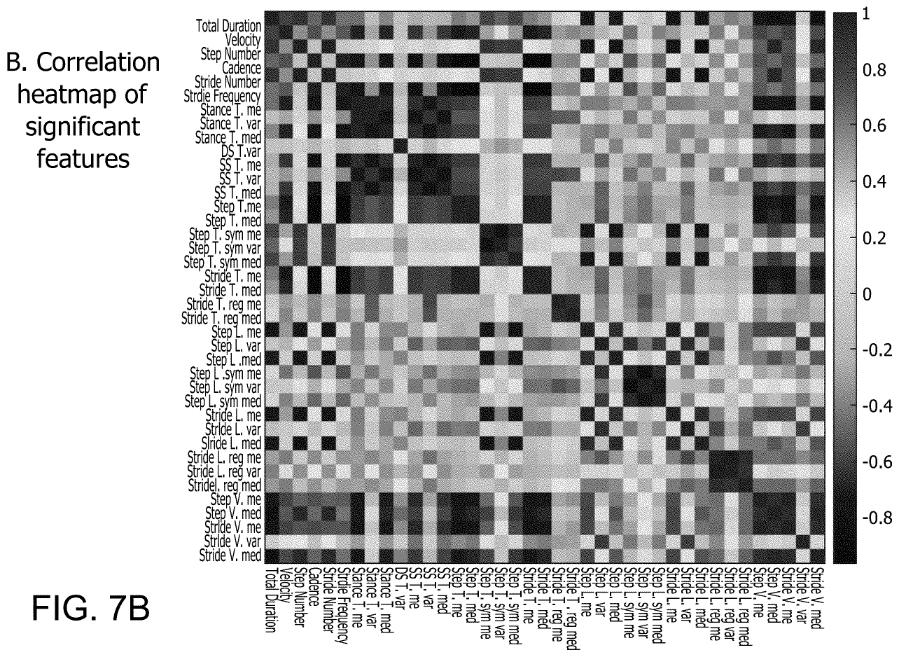

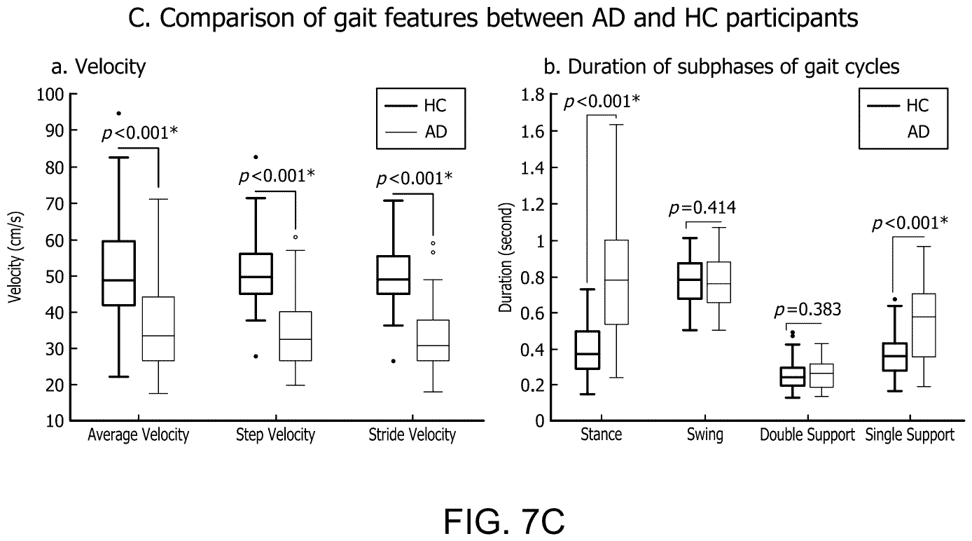

Significant and Selected Features for AD Detection: Following the adjustment of gait features for age, weight, and education levels, the descriptive statistical analysis identified significant gait markers between HC and AD. The statistical examination revealed that out of forty-eight gait features analyzed from the ten-meter curved walking test, thirty-nine exhibited significant differences between the AD and HC groups. These included various features: six macro, sixteen micro temporal, twelve micro spatial, and five spatiotemporal gait features (see the top left portion of FIG. 7A). Subsequent correlation analysis among these significant features pinpointed a subset with high correlation (r>0.9), leading to a refined selection based on their diagnostic power as shown in the right portion of FIG. 7B. Notably, mean step time emerged as a key indicator due to its profound significance in distinguishing between AD and HC subjects (with the lowest p-value=1.21*10-22), reducing the number of significant gait features to eighteen unique markers. These markers comprise a comprehensive profile of gait dynamics, including macro, micro temporal, micro spatial, and micro spatiotemporal features, which collectively enhance the accuracy of AD detection.

Moreover, the study highlights a weaker gait performance in older adults with AD than HC during the ten meter curved path walking test. AD participants exhibited notably lower average velocity and cadence, requiring more time to complete the assessment. Specifically, the average velocity and cadence for the AD group were 35.29±19.42 cm/s and 75.90±19.33 steps/minute, respectively, versus 51.15±16.67 cm/s and 88.76±12.36 steps/minute for the HCs, with a statistically significant difference (p-value<0.001, see the bottom left portion of FIG. 7C).

This trend extends to micro-gait features. AD individuals demonstrated longer durations in various subphases of gait cycles and reduced step and stride lengths and velocities, significantly impacting their mobility and balance. Notably, the duration for stance and single support phases was significantly higher in the AD group, with durations of 0.78±0.39 s and 0.55±0.23 s, respectively, compared to 0.40±0.19 s and 0.37±0.15 s in HCs (p-value<0.001; see FIG. 7C). Additionally, mean step and stride lengths in AD participants were significantly shorter, at 23.89±5.55 cm and 47.98±11.20 cm, respectively, compared to 30.68±4.81 cm and 61.79±9.30 cm in HCs. A similar reduction was observed in step and stride velocity, with micro spatiotemporal features decreased to 34.31±11.92 cm/s and 32.82±12.02 cm for AD in comparison to 50.99±10.91 cm/s and 49.90±9.86 cm/s for ADs (see FIG. 7C). These findings highlight the profound impact of AD on gait dynamics, with marked differences in velocity, cadence, and gait cycle subphases serving as potential indicators of AD progression.

Classification of Participants into AD and HC Groups: The SVM algorithm was employed to classify participants into AD or HC groups based on the selected gait features. The classification process included training, validation, and testing phases, with the outcomes presented in the following TABLE 2, where various evaluation metrics such as accuracy, sensitivity, precision, and F-score were used to assess performance. The SVM achieved an impressive accuracy rate of 90.01%, along with a sensitivity of 87.00%, precision of 85.63%, and an F-score of 86.20% on the test dataset.

TABLE 2
Classification results utilizing gait features
with SVM to differentiate between AD and HC

| | Evaluation metrics (%) | | | | |
|---|---|---|---|---|---|
| Dataset | Accuracy | Sensitivity | Precision | Specificity | F-score |
| Training | 99.55 | 99.10 | 100.00 | 100.00 | 99.54 |
| Validation | 92.09 | 88.37 | 97.22 | 98.82 | 92.91 |
| Test | 90.01 | 87.00 | 85.63 | 93.02 | 86.20 |

Discussion: In advancing AD detection, the research emphasized the application of imaging techniques, specifically through gait analysis on a ten meter curved path, integrated with machine learning for precise diagnostics. A simplified yet effective system was employed utilizing a single camera and the human pose estimation the open-source algorithm for accurate pose estimation. This approach not only achieves cost-efficiency and ensures privacy protection but also capitalizes on the diagnostic potential of curved path walking. This walking pattern may uncover more nuanced insights into AD than a straight-line gait analysis due to its closer resemblance to everyday walking behaviors. By focusing on such naturalistic movement patterns, the present method stands to improve the applicability and accuracy of AD detection across a diverse elderly population, accommodating various education levels and promoting wider adoption in both clinical and research settings.

Findings: The exploration into gait analysis via a 10-meter curved path, employing a single camera alongside the open-source algorithm for body joint detection in older adults, has illustrated that tracking body movements during gait assessments can be conducted with notable accuracy. Despite encountering some challenges with full occlusion, particularly in the upper body parts obscured by other segments (see FIG. 6), the overall precision in capturing body joint movements was sufficient for effective gait feature extraction for AD detection.

Further analysis revealed clear performance differences between older adults diagnosed with AD and HCs. Specifically, AD individuals exhibited reduced velocities and increased durations in several gait cycle subphases during the curved path walking test as depicted in FIG. 7. These findings confirm the utility of the approach in identifying key gait alterations associated with AD.

Moreover, the study extended to assess the effectiveness of machine learning by applying SVM to the extracted gait features. This analysis objectively classified participants into AD or HC categories based on their gait characteristics. The results, bolstered by a 5-fold validation process, demonstrated the SVM's robust ability to differentiate between AD and HC groups, achieving an accuracy and F-score of 90.01% and 86.20%, respectively, supporting the potential of integrating imaging and machine learning for enhancing the precision of AD detection in clinical settings (see TABLE 2).

Clinical Implications, Study Limitations, and Future Directions: The developed screening tool for AD demonstrates significant advantages, including its non-invasiveness, ease of setup, cost-effectiveness, and minimal intrusiveness, compared to other diagnostic methods and earlier technological approaches. By employing just a regular camera, such as those found in smart phones, to record curved-path walking, the present method sidesteps the need for large spaces or complex cognitive tasks that might challenge older adults, especially those with limited educational backgrounds. The method's capability to extensively extract and analyze gait features, identifying those uniquely indicative of AD, simplifies and enhances the accuracy and speed of clinical diagnoses.

Summary: The above experiment effectively showcased image analysis, particularly HPE employing CNNs, as a means for proactively identifying AD. Utilizing standard camera technology in conjunction with the open-source algorithm, an economical and accessible modality has been formulated for AD diagnosis, analyzing gait dynamics during a 10-meter curved walking examination. Demonstrating substantial efficacy, the present methodology differentiates HC from those with AD with an accuracy of 90.0% and an F-score of 86.20%. The innovative use of a singular camera and the open-source algorithm's intricate image analysis capabilities facilitated the derivation of twenty-five unique body joint coordinates. Forty-eight gait features were extracted by analyzing these, uncovering marked differences in thirty-nine gait features between the HC and AD cohorts. These outcomes support the utility of gait analysis as a potent biomarker in AD diagnostics.

### Second Experimentation: Detection of Mild Cognitive Impairment Using Various Types of Gait Tests and Machine Learning.

This study explored the utility of analyzing gait patterns, a fundamental aspect of human motor behavior, on straight and oval paths for diagnosing MCI. Using a depth camera, we recorded the movements of twenty-five body joints from twenty-five individuals with MCI and thirty healthy older adults (HC). Signal processing, descriptive statistical analysis, and machine learning techniques were employed to analyze the skeletal gait data in both walking conditions.

The study demonstrated that both straight and oval walking patterns provide valuable insights for MCI detection, with a notable increase in identifiable gait features in the more complex oval walking test. The Random Forest (RF) model excelled among various algorithms, achieving an 85.50% accuracy and an 83.90% F-score in detecting MCI during oval walking tests. This research introduces a cost-effective, depth camera-based method that integrates gait analysis—a key behavioral pattern—with machine learning, offering a practical tool for MCI screening in both clinical and home environments.

Participants: A total of fifty-five adults sixty years or older, including thirty HC without any cognitive impairment and twenty-five older adults with MCI, were enrolled in the present study. The inclusion criteria for the participants were people who could perform the gait tests independently. Also, they had no stroke or knee or hip displacements, which could affect their common gait patterns. The demographic and clinical information of the participants in this study who completed all the gait tests is presented in following TABLE 3.

TABLE 3
Demographic and clinical information of participants

| Characteristic | HC (N = 30) | MCI (N = 25) | p-value |
|---|---|---|---|
| Age (years) | 68.33 ± 2.15 | 69.76 ± 6.45 | 0.091 |
| Female gender, N (%) | 18 (60) | 19 (76) | 0.216 |
| BMI (kg/m²) | 24.51 ± 2.67 | 26.67 ± 2.62 | <0.001* |
| Years of education | 13.53 ± 3.05 | 11.56 ± 3.00 | 0.008* |
| MMSE | 28.50 ± 1.17 | 25.60 ± 1.29 | <0.001* |
| MoCA | 27.13 ± 2.05 | 22.76 ± 1.69 | <0.001* |
| GDS | 1.43 ± 1.33 | 3.52 ± 1.29 | <0.001* |

Mean ± standard deviation was shown. N, Number of participants; HC, Healthy Cognitive Control Group; MCI, Mild Cognitive Impairment; BMI, Body Mass Index; MMSE, Mini-Mental State Examination (maximum score, 30); MoCA, Montreal Cognitive Assessment (maximum score, 30); GDS, Geriatric Depression Scale (maximum score, 15). The gait feature analysis was adjusted for the confounding variables of BMI, education, and GDS.
*Significant difference at p-value < 0.05.

Gait measurements: The ten meter walking test, a standard gait test at clinics, was recorded from the participants in the single-cognitive task condition for straight and oval paths. The participants were given instructions before performing these gait tests, and they could practice the test three times before the trial recording process. The participants walked at their preferred gait speed without support from the recording and healthcare team and did the gait tests independently. The participants rested up to five minutes between the recordings as they needed. Participants selected their preferred direction—clockwise or counterclockwise—for the oval walking test, enhancing the authenticity of gait data by accommodating natural walking tendencies and reducing potential performance anxiety.

A single depth camera was connected to a laptop. The depth camera was mounted on a tripod next to the recording paths, and the recording process was controlled by a graphical user interface (GUI). The depth camera can detect and track twenty-five joints of the participant's body and record the RGB and depth data. The accuracy of pose estimation with depth camera is influenced by the type of activity, camera setup, and environmental factors.

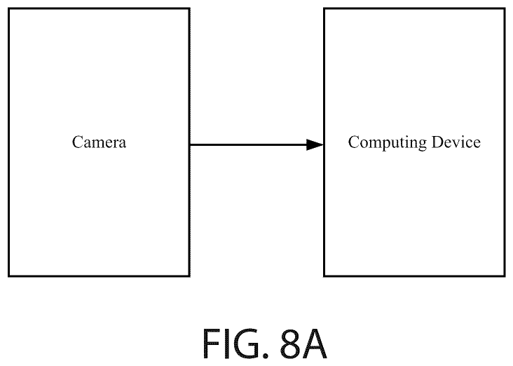

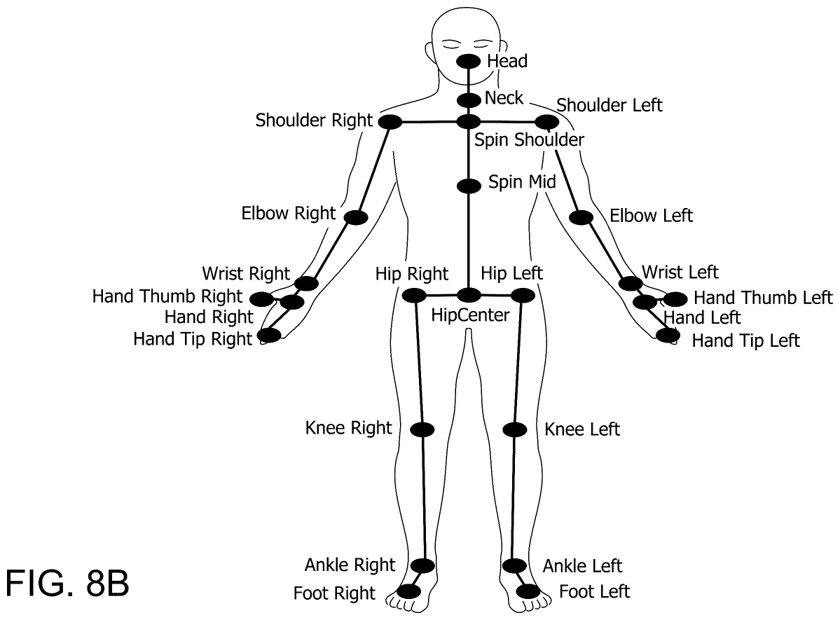

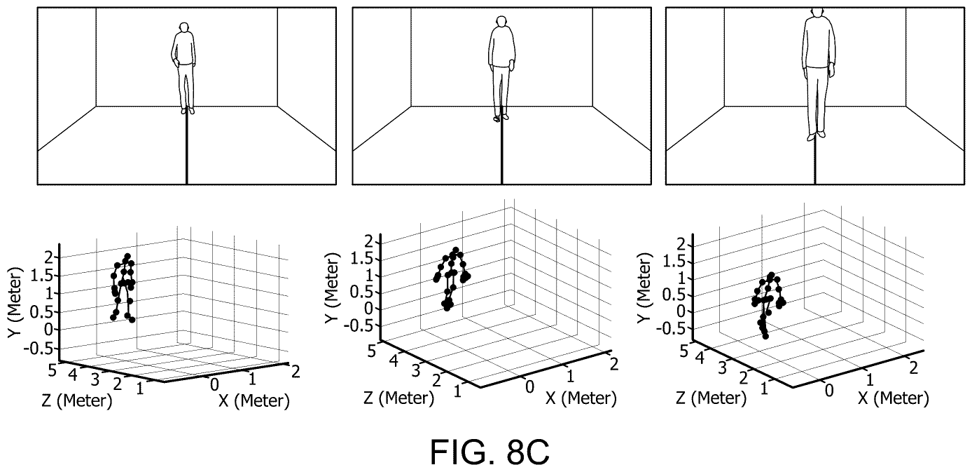

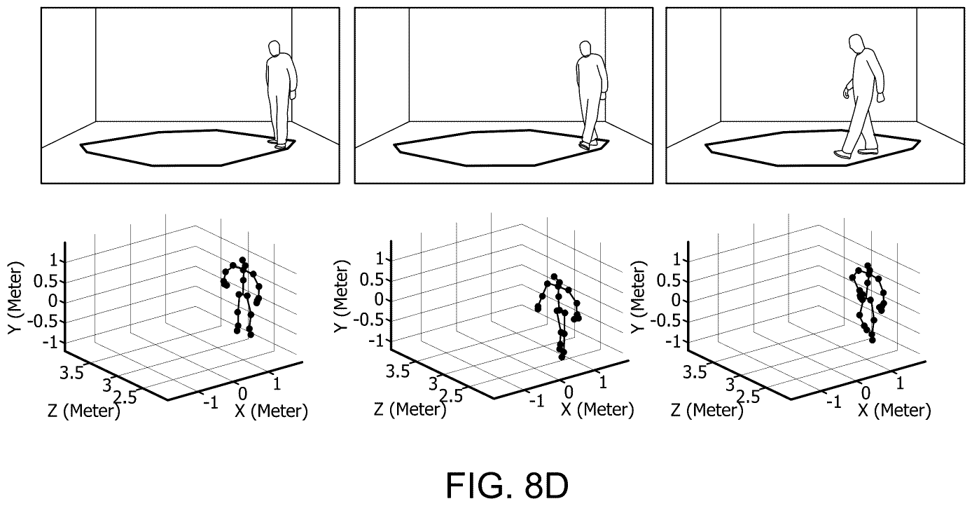

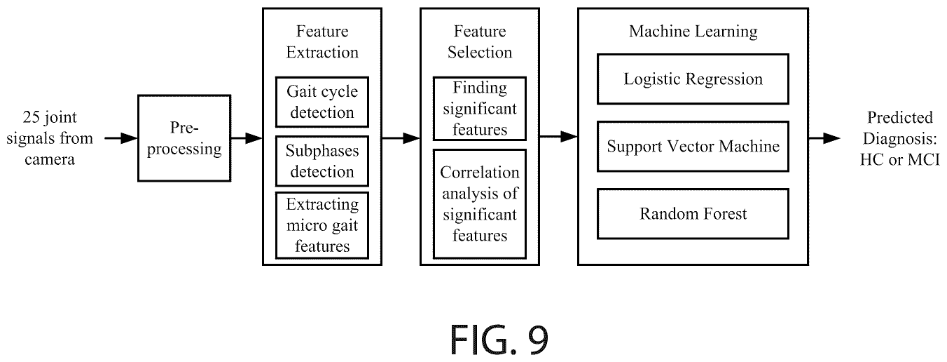

FIG. 8 shows the recording tools and settings, and twenty-five of the body's joints can be detected and tracked by the depth camera. In this study, only the skeletal data was used, which are the signals of movements of the body joints detected and tracked by the depth camera for further gait analysis. The skeletal data was used because this type of data can provide comprehensive information about the movement of twenty-five body joints, as seen in FIG. 8B, while the privacy issue and the high-cost hardware for the following analysis are solved. FIGS. 8C-8D show the samples of recorded RGB and skeletal data from a participant while performing straight and oval walking tests, respectively.

In FIG. 8, the camera was placed frontally for straight-path walking to ensure clear, unobstructed data capture and laterally for oval-path walking to manage space constraints and minimize self-occlusion issues associated with lateral movements. It should be noted that healthy controls and MCI participants underwent gait analysis under identical camera setups for straight and oval path walking tests. This ensured that any differences observed in detecting MCI were due to genuine gait variations and not influenced by differences in camera placement. This analysis examined the effect of camera view changes on estimating ankle-foot joint distances. The results showed no significant difference in joint distance measurements between the camera views, confirming that the observed differences in gait features between HC and MCI participants are not attributable to camera placement biases.

Data processing: After recording data, a comprehensive analysis was done on the skeletal data using signal processing algorithms, descriptive statistical analysis, and machine learning tools. These processes included prepossessing, feature extraction, feature selection, and participant classification using different machine learning algorithms. FIG. 2 shows the general steps of the proposed algorithm for data processing of the recorded data, which are described in more detail in the following.

Preprocessing: The location changes of the twenty-five body joints, which were detected and tracked by the depth camera for gait tests, can be presented as the signals during time. In the preprocessing step, a six-order Butterworth filter with a cut-off frequency of 3 Hz was applied to remove noises from the movement signals of the body's joints.

Feature extraction: A comprehensive collection of fifty gait features (see below TABLE 4) were extracted from the preprocessed signals of the body's joints for separate straight and oval path walking tests. The feature extraction algorithms included several steps. For macro features like average velocity, which shows the general performance of the participant for the gait test, the total displacement of the foot joint was used from the starting to the end point of the walking tests, and it was divided by the duration needed to finish the test. However, for the forty-nine remaining extracted features, the system was used to detect the gait cycles and their subphases (e.g., step, stance, swing phases, etc.) and calculate the features (e.g., step time, step length, swing time, etc.). These types of features are usually called micro features as they can provide more detail about the gait performance of older adults.

TABLE 4

| Feature | | Straight Walking | | | Oval walking | | |
|---|---|---|---|---|---|---|---|
| Type | Name | HC | MCI | p-value | HC | MCI | p-value |
| Macro | Velocity | 50.12 ± 27.92 | 47.39 ± 9.80 | 0.780 | 45.90 ± 8.89 | 39.27 ± 10.09 | 0.007* |
| | Cadence | 68.22 ± 10.55 | 68.90 ± 9.70 | 0.247 | 62.02 ± 9.73 | 55.00 ± 11.67 | 0.018* |
| Micro | StanceT.me (s) | 0.78 ± 0.24 | 0.83 ± 0.34 | 0.716 | 0.57 ± 0.15 | 0.64 ± 0.22 | 0.138 |
| Temporal | Stance T.var (%) | 18.15 ± 20.91 | 23.48 ± 26.30 | 0.993 | 49.91 ± 18.75 | 73.08 ± 20.27 | <0.001* |
| | StanceT.med (s) | 0.78 ± 0.24 | 0.84 ± 0.33 | 0.729 | 0.53 ± 0.17 | 0.52 ± 0.23 | 0.384 |
| | SwingT me (s) | 0.71 ± 0.15 | 0.72 ± 0.16 | 0.841 | 0.54 ± 0.04 | 0.52 ± 0.07 | 0.211 |
| | SwingT.var (%) | 12.41 ± 16.09 | 19.83 ± 13.89 | 0.053 | 26.10 ± 8.00 | 32.89 ± 11.12 | 0.018* |
| | Swing. med (s) | 0.69 ± 0.15 | 0.72 ± 0.16 | 0.742 | 0.51 ± 0.05 | 0.48 ± 0.06 | 0.001* |
| | DST.me (s) | 0.21 ± 0.11 | 0.25 ± 0.14 | 0.323 | 0.21 ± 0.04 | 0.31 ± 0.09 | <0.001* |
| | DST.var (%) | 69.63 ± 38.82 | 80.65 ± 26.45 | 0.407 | 80.16 ± 22.57 | 93.91 ± 21.54 | 0.072 |
| | DST.med (s) | 0.13 ± 0.08 | 0.17 ± 0.10 | 0.080 | 017 ± 0.05 | 0.23 ± 0.05 | <0.001* |
| | SST.me (s) | 0.53 ± 0.16 | 0.52 ± 0.15 | 0.498 | 0.40 ± 0.08 | 0.35 ± 0.09 | 0.014* |
| | SST.var (%) | 37.30 ± 23.06 | 45.14 ± 25.72 | 0.196 | 49.00 ± 13.07 | 48.56 ± 11.29 | 0.896 |
| | SST.med (s) | 0.57 ± 0.18 | 0.55 ± 0.18 | 0.496 | 0.36 ± 0.09 | 0.34 ± 0.10 | 0.158 |
| | StepT.me (s) | 0.77 ± 0.13 | 0.78 ± 0.11 | 0.331 | 0.88 ± 0.13 | 1.05 ± 0.27 | 0.012* |
| | StepT.var (%) | 18.05 ± 16.60 | 27.13 ± 11.29 | 0.042* | 34.08 ± 17.07 | 40.51 ± 10.50 | 0.017* |
| | StepT.med (s) | 0.76 ± 0.09 | 0.77 ± 0.10 | 0.606 | 0.83 ± 0.09 | 0.94 ± 0.20 | 0.013* |
| | StepT.sym me | 0.64 ± 0.08 | 0.64 ± 0.06 | 0.680 | 0.83 ± 0.03 | 0.84 ± 0.03 | 0.207 |
| | StepT.sym vat(%) | 24.22 ± 20.21 | 34.77 ± 14.35 | 0.035* | 14.64 ± 5.93 | 14.45 ± 5.27 | 0.899 |
| | StepT.sym med | 0.68 ± 0.08 | 0.70 ± 0.08 | 0.452 | 0.87 ± 0.03 | 0.88 ± 0.03 | 0.019* |
| | StrideT.me (s) | 1.55 ± 0.30 | 1.54 ± 0.22 | 0.254 | 1.77 ± 0.26 | 2.08 ± 0.53 | 0.016* |
| | StrideT.var (%) | 13.66 ± 14.58 | 19.61 ± 9.54 | 0.049* | 26.12 ± 12.36 | 29.41 ± 10.98 | 0.261 |
| | StrideT.med (x) | 1.57 ± 0.30 | 1.58 ± 0.25 | 0.496 | 1.67 ± 0.18 | 1.92 ± 0.42 | 0.024* |
| | StrideT.reg me | 0.94 ± 0.06 | 0.77 ± 0.12 | <0.001* | 0.76 ± 0.09 | 0.74 ± 0.09 | 0.414 |
| | StrideT.reg var (%) | 0.37 ± 2.35 | 5.73 ± 10.14 | 0.118 | 21.75 ± 14.39 | 18.65 ± 9.16 | 0.729 |
| | StrideT.reg med | 0.94 ± 0.06 | 0.77 ± 0.12 | <0.001* | 0.75 ± 0.11 | 0.74 ± 0.11 | 0.676 |
| Micro | StepI.me (cm) | 35.75 ± 5.53 | 31.06 V 6.74 | 0.003* | 37.58 ± 4.97 | 32.91 ± 3.46 | <0.001* |
| Spatial | StepI.var (%) | 21.21 ± 18.29 | 32.65 ± 19.51 | 0.016* | 37.01 ± 9.97 | 41.84 ± 9.57 | 0.075 |
| | StepI.med (cm) | 37.63 ± 5.12 | 30.99 ± 9.19 | <0.001* | 40.16 ± 5.76 | 34.22 ± 4.90 | <0.001* |
| | StepI.sym me | 0.80 ± 0.17 | 0.74 ± 0.19 | 0.106 | 0.70 ± 0.10 | 0.66 ± 0.11 | 0.114 |
| | StepI.sym var (%) | 22.16 ± 31.41 | 21.88 ± 15.86 | 0.174 | 34.87 ± 12.43 | 38.87 ± 13.25 | 0.255 |
| | StepI.sym med | 0.83 ± 0.18 | 0.75 ± 0.19 | 0.025* | 0.75 ± 0.13 | 0.67 ± 0.16 | 0.030* |
| | StepW.me (m) | 0.12 ± 0.03 | 0.13 ± 0.03 | 0.919 | 0.21 ± 0.04 | 0.17 ± 0.03 | 0.002* |
| | StepW.var (%) | 30.91 ± 12.54 | 23.10 ± 11.13 | 0.013* | 56.82 ± 12.33 | 67.09 ± 13.87 | 0.011* |
| | StepW.med (m) | 0.13 ± 0.03 | 0.13 ± 0.03 | 0.912 | 0.21 ± 0.06 | 0.16 ± 0.04 | 0.022* |
| | StepH.me (m) | 0.11 ± 0.04 | 0.08 ± 0.04 | 0.003* | 0.12 ± 0.02 | 0.13 ± 0.02 | 0.069 |
| | StepH.var (%) | 28.56 ± 12.18 | 39.24 ± 14.15 | 0.012* | 38.65 ± 14.60 | 46.29 ± 11.20 | 0.004* |
| | StepH.med (m) | 0.11 ± 0.04 | 0.08 ± 0.05 | 0.020* | 0.11 ± 0.02 | 0.12 ± 0.02 | 0.114 |
| | StrideI.me (cm) | 74.02 ± 11.16 | 63.62 ± 14.92 | 0.006* | 75.84 ± 10.44 | 65.91 ± 6.83 | <0.001* |
| | StrideI.var (%) | 11.82 ± 14.75 | 21.45 ± 13.08 | 0.013* | 27.84 ± 11.09 | 30.83 ± 12.33 | 0.393 |
| | StrideI.med (cm) | 74.36 ± 12.32 | 63.88 ± 15.35 | 0.002* | 76.66 ± 11.61 | 63.10 ± 9.50 | <0.001* |
| | StrideI.reg me | 0.96 ± 0.06 | 0.79 ± 0.13 | <0.001* | 0.68 ± 0.12 | 0.70 ± 0.12 | 0.657 |
| | StrideI.reg var (%) | 2.44 ± 2.72 | 6.60 ± 14.42 | 1.00 | 23.37 ± 15.17 | 24.66 ± 14.71 | 0.666 |
| | StrideI.reg med | 0.96 ± 0.06 | 0.79 ± 0.13 | <0.001* | 0.68 ± 0.13 | 0.71 ± 0.15 | 0.369 |
| Micro | StepV.me (cm/s) | 47.26 ± 7.04 | 45.62 ± 13.01 | 0.574 | 46.41 ± 8.05 | 37.17 ± 8.08 | <0.001* |
| spatial | StepV.var (%) | 30.75 ± 19.45 | 52.01 ± 21.87 | <0.001* | 47.66 ± 9.92 | 53.47 ± 14.37 | 0.083 |
| temporal | StepV.med (cm/s) | 47.66 ± 8.28 | 41.67 ± 12.88 | 0.139 | 46.88 ± 9.68 | 35.08 ± 9.92 | <0.001* |
| | StrideV.me (cm/s) | 48.08 ± 7.22 | 43.65 ± 12.81 | 0.177 | 45.51 ± 7.91 | 35.47 ± 8.51 | <0.001* |
| | StrideV.var (%) | 14.47 ± 14.88 | 28.80 ± 13.21 | 0.001* | 37.78 ± 11.52 | 38.89 ± 10.05 | 0.704 |
| | StrideV.med (cm/s) | 48.13 ± 7.97 | 43.16 ± 12.73 | 0.165 | 46.35 ± 8.48 | 34.15 ± 10.06 | <0.001* |

Data are shown as mean ± standard deviation; p-value is reported for comparison between two study groups;
*Significant difference at p-value < 0.05.
HC, healthy control without cognitive impairment; MCI, mild cognitive impairment; T, time; L, length; W, width; H, height; V, velocity; me, mean; var, variability; sym, symmetry; reg, regularity; DS, double support; SS, single support.

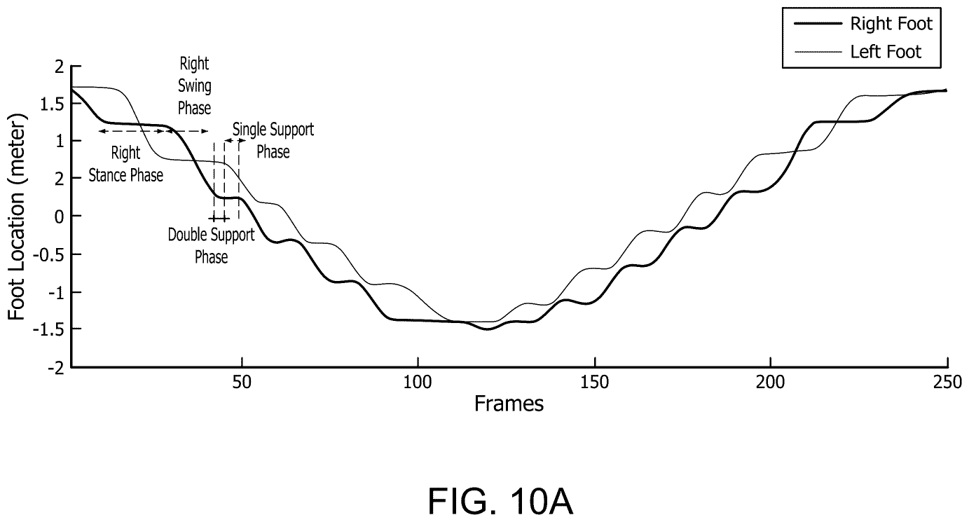

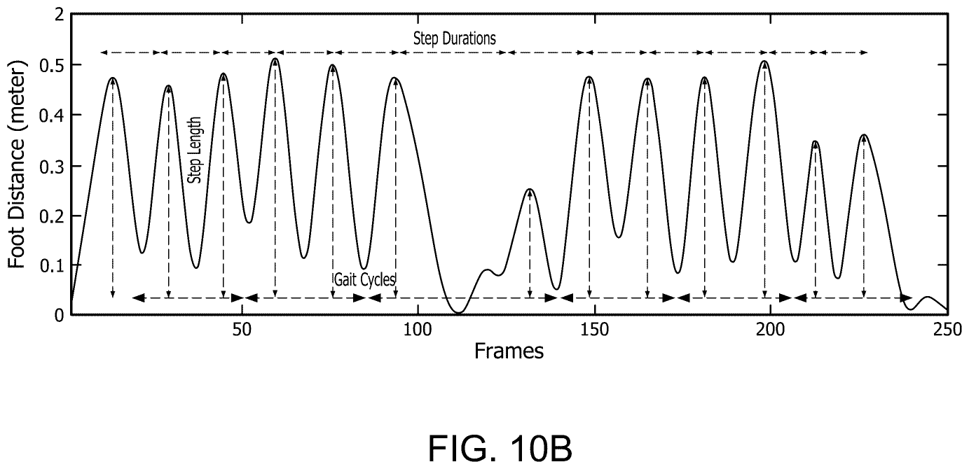

Several analyses were done on the right and left foot joints to extract the micro features. FIG. 10 shows the plotted signals of the right and left foot and their distance signal for one of the participants while performing the gait test. First, the gait cycle was detected by plotting the distance signals of the right and left feet and their peaks. Each gait cycle is defined as the duration a foot contacts the ground to when the same foot again contacts the ground. Each gait cycle comprises two successive steps; two successive steps are also known as stride or a single gait cycle. Thus, the distance signal of the right and left feet was created, and then used the duration between two successive peaks or valleys of this signal to find the gait cycles (see FIG. 10B). Each peak of the distance signal of the feet shows the steps' location and length (see FIG. 10B). To extract the features from the subphases of the gait cycles, including the stance time, swing time, single support time, and double support time, the derivative of the right and left foot signals was used. For the stance subphase of one leg, the foot has no location changes and remains in contact with the ground (see FIG. 10A). Thus, its derivative of the foot signal is approximately zeros. In comparison, for the swing phase of a foot, the derivative of the movement signal is not zero because the foot's location changes. For the subphases of single support, the location of one of the feet changes, and the other foot does not change, while in the double support phase, both of the feet locations do not change (see FIG. 10A). Thus, in the single support, the derivative of one foot is zero, and the other is non-zero, while in the double support phase, the derivative of signals for both feet’ movement are zeros.

Graph of FIG. 10A shows the gait cycles and their subphases extracted from the signals of the body's joints during walking. After detecting the various subphases of gait cycles, statistical metrics, including the mean, median, and variability of the extracted subphases, were calculated to provide information about the performance of the participants during the whole process of gait tests. Various statistical metrics were calculated for micro gait features consisting of mean, median, variability, symmetry, or regularity. A feature's variability is calculated by dividing the standard deviation of a feature by its mean, which can be presented using the percentage. Mathematical equation (2) shows the variability of micro features.

$$var ( x ) = \frac{std ( x )}{mean ( x )} * 100 \qquad ( 2 )$$

var(x) and std show the variability and standard deviation of a micro feature of x, respectively. The symmetry and regularity of step and stride features show the similarity between the right and left feet during walking. Mathematical equation (3) shows this similarity index.

$$SI ( x ) = 1 - \frac{\vert {x}_{right} - {x}_{left} \vert}{max ( {x}_{right} , {x}_{left} )} \qquad ( 3 )$$

SI(x) shows the index of symmetry or regularity of features x for step or stride during walking. xright and xleft are the values of the gait features for right and left feet, respectively. The range of this index can change from zero to one. The higher value of SI(x) means higher symmetry or regularity for the x feature.

Feature selection: Before determining the significant features, an investigation was performed of the data for any confounding factors. The demographic and clinical information of the participants, including age, BMI, years of education, and neuropsychological test scores, were compared between two study groups using descriptive statistical analysis methods. Shapiro-Wilk was used for normality check. Unpaired t-test and Mann-Whitney U tests were applied for normally distributed and non-normally distributed demographic and clinical information, which were numerical variables. For categorical variables like gender, the chi-square test was used. The analysis showed significant differences between the two study groups for confounding variables of BMI, years of education, and GDS scores, which can affect the gait tests. Thus, the extracted gait features were adjusted using ANCOVA and removed the effect of those confounding variables.

A feature selection was conducted separately for straight walking gait markers and oval path markers to distinguish between MCI and HC participants. Two strategies were employed: statistical methods commonly used in clinical research and machine learning techniques.

Statistical approach: The process involves a two-step method. Initially, an evaluation is performed on each gait feature for statistical significance. The normality of each feature is assessed using the Shapiro-Wilk test. Features conforming to a normal distribution are analyzed with the unpaired t-test. At the same time, those who do not meet this criterion are evaluated using the Mann-Whitney U test, with a significance threshold set at p<0.05. This first phase identifies features with significant differences between the study groups. A correlation analysis was conducted among the significant features in the second phase. For any set of features demonstrating a correlation coefficient greater than 90%, the feature was selected with the lowest p-value. This strategy reduces redundancy and concentrates on the most discriminative features, thereby aiding clinical experts by highlighting essential gait biomarkers for MCI detection. This refined set of features also simplifies the input for classification methods, potentially enhancing the accuracy and efficiency of tools like Logistic Regression (LR) and SVM.

Machine learning approach: we also utilized an RF algorithm. The RF was applied to all extracted features from the oval and straight-walking datasets without preliminary selection. This approach allows the RF model itself to determine the importance of each feature, offering an unbiased insight into which features most effectively differentiate between MCI and HC.

Machine learning methods: In this study, three different machine learning models were used to discriminate the MCI and HC participants using selected gait features from straight and oval path walking tests separately. The three classifiers were LR, SVM, and RF to classify participants into MCI and HC. These different types of classifiers were selected because each of them uses a different method for finding the decision boundary between two study groups.

Logistic regression: LR is a straightforward and highly effective classifier for binary and linear classification challenges. Despite its simplicity, this classifier demonstrates remarkable efficacy in addressing binary problems and is frequently employed in medical and clinical investigations. LR is a transformation of a linear regression using a sigmoid function. The input of the logistic function is the vector of features, while its output is the output of a Sigmoid function ranging from 0 to 1. Mathematical equation (4) shows the formula of the LR classifier.

$$f ( X ) = \frac{1}{1 + {e}^{{w}_{0} + {w}_{1} {x}_{1} + … + {w}_{n} {x}_{n}}} \qquad ( 4 )$$

X=x1, . . . , xn shows the vectors of the input features, and the f(X) shows the output of the sigmoid function. W=w1, . . . , wn are the weights or parameters of the LR classifier, which are optimized using the training and validation data to fit the generalized models on the data and then applied to the new test data.

Support vector machine: SVM represents a supervised binary classifier commonly advocated for the analysis of clinical data, particularly when the dimensionality of the features exceeds the number of available samples. This classifier endeavors to distinguish between two study groups by delineating a linear hyperplane, and in cases where the data is not linearly separable, a transformation into a new space is achieved using kernels. Mathematical equation (5) shows mapping samples of Xi and Xj to a new feature space using the map φ.

$$K ( {X}_{i} , {X}_{j} ) = ( Φ ( {X}_{i} ) \text{?} Φ ( {X}_{j} ) ) \qquad ( 5 )$$

$$\text{?} \text{indicates text missing or illegible when filed}$$

K is the kernel for mapping of Xi and Xj samples. To simplify the mapping process, particular kernels like linear, polynomial, or Radial Basis Function (RBF) are usually used in practical problems. After mapping features to a space where the data can be separated linearly, the optimal weights for a separable hyperplane with the maximum margin are found using the training data. Mathematical equation (6) shows the final nonlinear decision function for the SVM classifier.

$$f ( X ) = sign ( \text{?} K ( \text{?} ) + b ) \qquad ( 6 )$$

$$\text{?} \text{indicates text missing or illegible when filed}$$

f(X) shows the decision (label) predicted for the sample of X. n is the dimension of the features for each sample of data. K is the kernel for mapping the data. W=w1, . . . , wn are the weights (coefficients) of the decision hyperplane, and b is the intercept. These hyperplane parameters are found using the training data to maximize the distance between the hyperplane and the training samples. The optimal hyperplane is called the maximal margin hyperplane and is used to make decisions about the test data.

The optimization of the SVM model for subsequent applications to test data involves the adjustment of certain parameters, such as the selection of the kernel type, its associated parameters, and regularization, all of which are fine-tuned utilizing the training and validation data in the training step.

Random forest: RF is a classifier that combines the output of several decision trees to achieve a single final decision. This algorithm is an extension of the bagging method, which uses bagging and feature randomness to create an uncorrelated forest of decision trees. Given a training set D=X1, . . . , Xm with the labels Y=y1, . . . , ym, bagging selects a random subset of training data for b=1, . . . , B (B times) to make different trees. The results of the B trees are combined with the majority voting for classification and averaging for the regression problem to predict the output for the test sample. Considering fb as a decision tree classifier and Ensemble E=f1, . . . , fB is the collection of classifiers. The decision of the bth classifier (fb) is denoted by db,j∈{0, 1} while j=1, 2, . . . , Q and k is the number of classes. The decision tree of fb will produce db,j=1 if that classifier predicts a class or label of j, and is db,j=0 otherwise. The final decision about sample X using majority voting of B classifiers can be shown by mathematical equation (7).

$$\hat{Y} = arg {max}_{j} ( {\sum }_{b = 1}^{B} \text{?} ) \qquad ( 7 )$$

$$\text{?} \text{indicates text missing or illegible when filed}$$

Y is the final predicted label using the majority voting. j and b show the available classes and tree classifiers. db,j shows the decision of each tree whether the sample belongs to class j or not.

The difference between the random forest and the decision tree is that the random subsets of features are generated in RF. In contrast, all the possible feature splits are considered in the decision tree. As the RF chooses subsets of features randomly, it ensures low correlation among the decision trees. The parameters of RF models, like the number of trees, the maximum depth of each decision tree, minimum samples per leaf, minimum samples per split, and maximum number of features for the best split, were found using the training and validation set of data during the training process.

Experiment setup: The 5-fold cross-validation methods were used for all the above classifiers to divide the available data to train and test data. To find the generalized model before applying it to the test data, the train data was split into 80% for training and 20% for validation. Also, the grid search strategy was used for all the classifiers to find the optimal parameters of the models. Various quantitative metrics of accuracy, sensitivity, precision, specificity, and F-score evaluated the results of the classifiers for discrimination of MCI and HC.

Results: The demographic and clinical information of the participants in this study who completed all the gait tests is presented in TABLE 2. There were significant differences in BMI and years of education between MCI and HC participants, but no differences in age or gender. Participants with MCI had higher levels of depression on the GDS score, with lower MMSE and MoCA than the HC group. To ensure the robustness of the findings, we adjusted the gait feature analysis for these confounding variables, including BMI, education, and GDS.

To examine the straight and oval path walking sensitivity for detecting MCI, we extracted a comprehensive collection of fifty features. This collection included two macro, twenty-four micro temporal, eighteen micro spatial, and six micro spatiotemporal features. TABLE 3 shows the extracted gait features for gait tests in different paths. The values of the gait features are presented as the mean, standard deviation, and the p-values of each gait feature for the comparison between the study groups.

Feature selection outcome; Statistical approach; Significant gait features: The comparative analysis of extracted gait features demonstrated significant differences between MCI and HC participants, which were more pronounced in oval path walking than in straight walking. Specifically, in the oval path conditions, twenty-seven out of fifty extracted features showed significant differences, whereas, in straight walking, only twenty features were significant. Notably, the significant features in oval walking included two macro features (average velocity and cadence) and various micro gait features twelve temporal, nine spatial, and four spatiotemporal. In contrast, straight walking yielded significant results primarily in micro gait features five temporal, thirteen spatial, and two spatiotemporal.

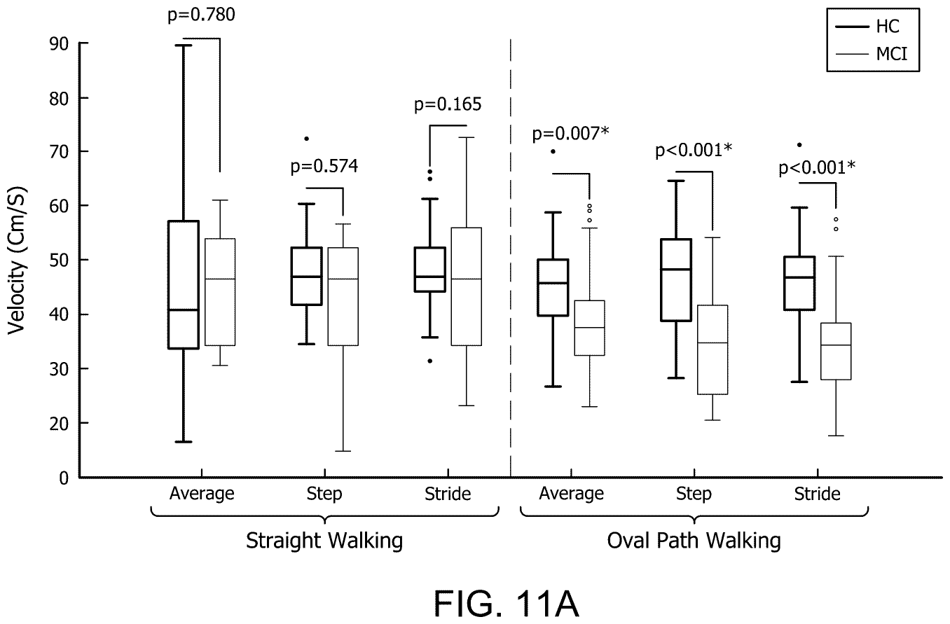

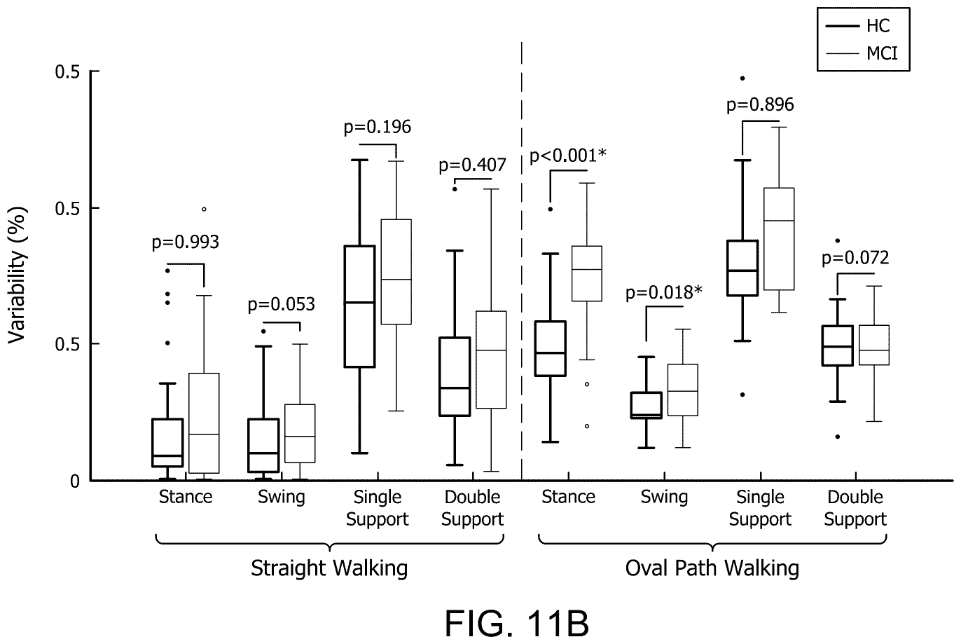

Macro features such as average velocity and cadence significantly differed in oval path walking, with MCI participants exhibiting lower average speed (39.27±10.09 cm/s) and cadence (55.00±11.67 steps/min) compared to HC participants (45.90±8.89 cm/s and 62.02±9.73 steps/min), with p-values of 0.007 and 0.018, respectively. Similar changes were observed for step and stride velocity between MCI and HC when the walking test changed from straight to oval path walking, and only the significantly lower step and stride velocity for MCI than HC were observed in oval path walking. This suggests a greater impact of the walking path shape on gait dynamics in MCI subjects. FIG. 11A compares the average velocity and the step and stride velocity between two study groups in straight and oval path walking conditions.

Furthermore, changes in gait cycle duration were observed when switching from straight to oval walking. For MCI participants, the gait cycle increased from 1.54±0.22 seconds to 2.08±0.53 seconds (p-value=0.016), compared to a more stable change from 1.55±0.30 seconds to 1.77±0.26 seconds among HC participants (p-value=0.254). Examining the subphases of the gait cycles provided more information on the participant's gait for comparison. Older adults with MCI generally had more variability for the subphases of the gait cycles, such as stance and swing time and the single and double support time. The differences between the variability of these subphases increased in oval walking, and even significant differences were observed for stance and swing times in oval path walking between MCI and HC participants (see FIG. 11B). This indicates a significant deterioration in gait coordination for MCI participants under more challenging walking conditions.

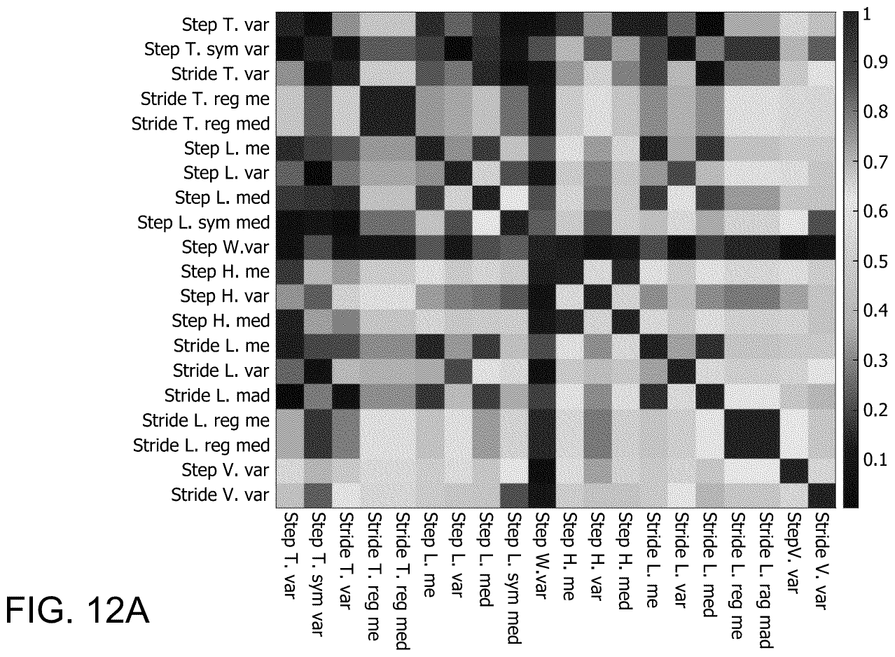

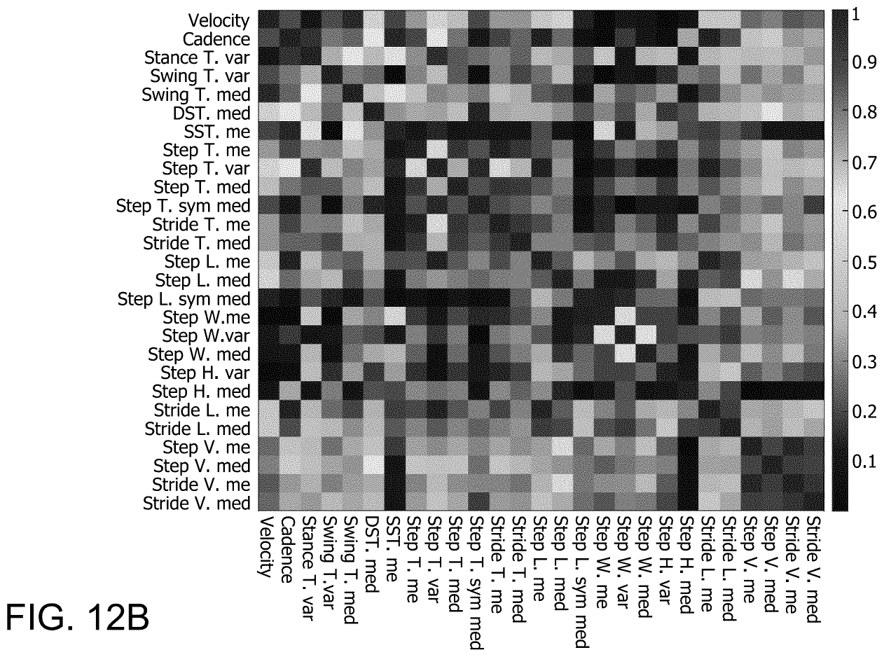

Selected gait features: Following the feature selection algorithm, a smaller set of unique gait features were considered that showed the most substantial differences between the two study groups without high inter-feature correlation. FIG. 12 illustrates this with heatmaps of correlation matrices for significant features from straight and oval path walking. This visualization confirms significant correlations among certain features, guiding the selection towards those with the lowest p-values, indicative of pronounced differences between MCI and HC participants.

Ultimately, from the oval path walking test, we selected nine-teen out of twenty-seven significant features, including two macro (average velocity and cadence), eight micro temporal (mean of single and double support time, the median of stance and swing time, and stride time, the variability of stance and swing time, and the median of the step time symmetry), seven micro spatial (mean of step length and width, the median of step and stride length and the symmetry of step length, and the variability of step height and width), and two micro spatiotemporal features (mean of step velocity and the median of stride velocity).

From straight walking, thirteen out of twenty significant features were retained, including three micro temporal (mean and median of stride time regularity and the variability of step time symmetry), eight micro spatial (mean of step height and stride length regularity, median of step length and stride length regularity, and the variability of step length, width, and height, and the stride length), and two micro spatiotemporal features (variability of step and stride velocity).

For the straight walking, the selected feature consisted of three micro temporal features (mean and median of stride time regularity and the variability of step time symmetry), eight micro spatial features (mean of step height and stride length regularity, median of step length and stride length regularity, and the variability of step length, width, and height, and the stride length), and two micro spatiotemporal features (variability of step and stride velocity). TABLE 5 summarizes the number of different types of extracted, significant, and selected features for straight and oval path walking.

TABLE 5

| | Ex- | Statistical approach | | | | | |
|---|---|---|---|---|---|---|---|
| | tract- | Significant | | Selected | | RF approach | |
| Type of feature | ed | Straight | Oval | Straight | Oval | Straight | Oval |
| Macro | 2 | 0 | 2 | 0 | 2 | 0 | 1 |
| Micro temporal | 24 | 5 | 12 | 3 | 8 | 3 | 7 |
| Micro spatial | 18 | 13 | 9 | 8 | 7 | 5 | 4 |
| Micro | 6 | 2 | 4 | 2 | 2 | 2 | 4 |
| spatiotemporal | | | | | | | |
| Total | 50 | 20 | 27 | 13 | 19 | 10 | 16 |

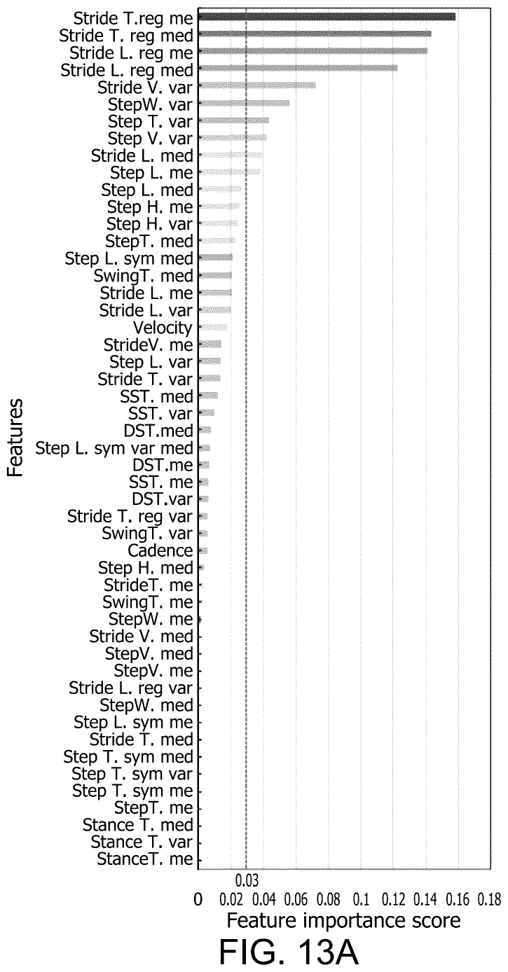

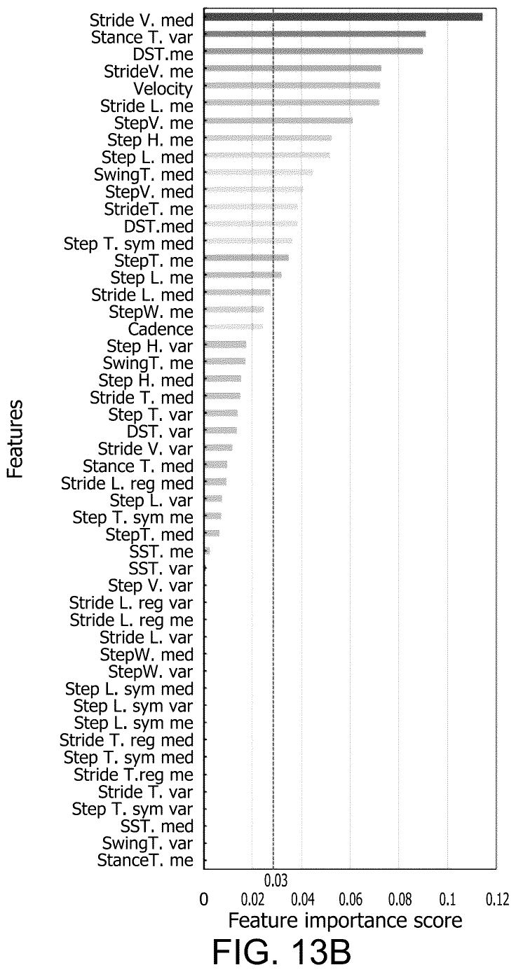

Machine learning approach: The RF algorithm was employed to assess the importance of gait features from straight and oval path walking conditions, each analyzed separately, to distinguish between MCI and HC participants. FIG. 13 displays the results from the RF classifier, highlighting the importance scores of gait features under both walking conditions. As illustrated in FIG. 13, the features garnered higher importance scores predominantly identified by the statistical approach, which utilized descriptive statistics and correlation analysis for feature selection. For instance, in straight walking (see FIG. 13A), the five features with the highest importance scores were the mean and median of stride time and length and variability of stride velocity. These features correspond to those selected through the proposed feature selection algorithm. Similarly, in oval walking, the top-ranked features by RF, such as the median of stride velocity, variability of stance time, mean of double support time, stride velocity, and average velocity (see FIG. 13B), aligned closely with those identified through the feature selection method. TABLE 5 lists the features with an RF importance score greater than 0.03.

MCI detection using machine learning: Three different classifiers were designed to discriminate the HC and MCI participants using selected features of straight and oval paths walking separately. It is important to note that for the SVM and LR classifiers, the statistical feature selection method was applied within a 5-fold cross-validation framework. This means that feature selection was conducted separately on the training data of each fold, ensuring that only the features identified from the training data were used to train the models, thereby preventing information leakage to the test data. The entire set of features for the RF classifier was used, allowing the algorithm to autonomously select the most predictive features within each cross-validation fold. 5-fold cross-validation was chosen because it balances computational efficiency and validation accuracy. This approach is optimal for the dataset size, reducing the risk of overfitting and variance compared to 10-fold or leave-one-out methods, which can be computationally intensive and less stable for moderate sample sizes.

As seen in TABLE 6, the classification accuracy of participants to MCI and HC groups using straight walking features were 65.8%, 76.1%, and 78.2% for LR, SVM, and RF, respectively. Also, the F-scores were 61.7%, 72.6%, and 75.9% for those classifiers in the same walking condition. In comparison, the results generally improved in oval path walking. The classification accuracy increased to 74.2%, 80.1%, and 85.50% for LR, SVM, and RF, respectively, when the features of oval path walking were used. Similar improvements were also seen for F-scores and other evaluation metrics, where the F-scores of these classifiers rose to 72.4%, 77.9%, and 83.9%. The comparison of the classification results using different classifiers and gait tests showed that the RF classifier on the oval path walking data had the best performance for discrimination of MCI and HC with accuracy and F-score of 85.5% and 83.9%. The other evaluation metrics, such as sensitivity, precision, and specificity, confirmed this finding, too.

TABLE 6

| | | Evaluation metrics (%) | | | | |
|---|---|---|---|---|---|---|
| | | | Sensi- | | Speci- | |
| Gait test | Classifier | Accuracy | tivity | Precision | ficity | F-score |
| Straight | LR | 65.8 | 60.0 | 63.6 | 71.7 | 61.7 |
| | SVM | 76.1 | 68.0 | 78.2 | 84.2 | 72.6 |
| | RF | 78.2 | 73.0 | 79.2 | 83.3 | 75.9 |
| Oval | LR | 74.2 | 75.0 | 70.1 | 73.3 | 72.4 |
| | SVM | 80.1 | 76.0 | 80.2 | 84.2 | 77.9 |
| | RF | 85.5 | 81.0 | 87.1 | 90.0 | 83.9 |

Discussion: Early detection of AD and dementia is pivotal in slowing or potentially preventing their progression to more severe stages, especially given the current lack of a cure for these degenerative diseases. Identifying MCI, a key precursor to AD, is therefore important. Individuals with MCI convert to AD at a higher annual rate than their cognitively healthy counterparts. Conventional clinical methods for MCI detection, including neuropsychological tests, brain imaging, EEG, and blood tests, are often time-consuming and costly, and the practitioner's experience can influence their effectiveness. To address these limitations, the study introduces a novel method for MCI detection employing comprehensive gait analysis during both oval and straight walking patterns, captured using a depth camera. This approach integrates signal processing, descriptive statistical tools, and machine learning techniques. The method presents an objective, non-invasive, and easy-to-implement alternative for MCI detection, offering a low-cost and less time-intensive solution suitable for both clinical and non-clinical settings. Data was collected from fifty-five older adults, comprising twenty-five individuals with MCI and thirty individuals with HC, to validate this approach.

Findings and implications: The study highlighted significant gait performance differences between MCI and HC groups. Older adults with MCI showed weaker performance, especially in oval path walking, resulting in more noticeable differences between MCI and HC groups than straight walking (see TABLE 4). In oval path walking, twenty-seven out of fifty features showed significant differences between MCI and HC, compared to twenty features in straight walking (see TABLE 6). Older adults with MCI demonstrated significantly lower average velocity and cadence during oval walking, while these macro gait features showed no significant differences in straight walking (see FIG. 11A). Similar trends were observed for step and stride velocity, with significant differences in oval walking but not straight walking conditions (see FIG. 11A). Analyzing gait subphases revealed increased variability in stance, swing, single support, and double support among MCI individuals, especially during oval walking, resulting in significant differences in stance and swing time between MCI and HC (see FIG. 11B). When walking along curves like an oval path, individuals with MCI face heightened cognitive demands, necessitating additional time to adapt to changes in direction and maintain their balance. Additionally, they exhibit more cautious movement along oval paths due to increased worries about falling, difficulties in spatial awareness, and intricate motor planning. The findings and this clinical evidence emphasize how gait features can detect the difficulties presented by various types of curved walking around an oval path and their potential role in indicating cognitive impairment. The study extends these findings, highlighting the higher sensitivity of oval walking compared to straight walking in detecting MCI.

Another observation was that was identified optimal gait features with enhanced sensitivity in distinguishing between MCI and HC through a two-step feature selection algorithm involving descriptive statistics and correlation analysis. This process yielded a reduced set of gait features suitable for efficient MCI detection in both oval and straight walking tests, with nine-teen features for oval walking and thirteen for straight walking (see FIG. 12 and TABLE 5). These selected features serve as focal points for clinicians, streamlining MCI detection and expediting machine learning procedures by eliminating redundant features. Further validation using the RF classifier corroborated the effectiveness of the feature selection methods. RF highlighted the most influential features for MCI detection, aligning with those identified through the selection algorithms (see FIG. 13).

Furthermore, various classifier models were employed to detect MCI in different oval and straight walking conditions, highlighting the higher sensitivity of oval walking for MCI detection. The RF model demonstrated superior performance among the classifiers. In oval walking, RF achieved an accuracy and F-score of 85.5% and 83.9%, while in straight walking, these metrics were slightly lower at 78.2% and 75.9% (Table 4). RF's enhanced performance can be attributed to its ensemble learning method, which combines multiple decision trees and aggregates their predictions, thereby reducing overfitting and enhancing model diversity.

Clinical implications: The study marks a pivotal advancement in the screening of MCI. Utilizing a depth camera, a non-invasive, cost-effective, and efficient method was developed to analyze gait patterns in both oval and straight walking tests. This technique stands out for its practicality in clinical settings, where it can serve as an initial screening tool for MCI. One advantage is detecting subtle gait irregularities, often early indicators of cognitive decline, that may not be evident in standard clinical evaluations. From a clinical perspective, this approach could significantly streamline the early detection process of MCI, facilitating prompt intervention. Integrating this method into standard geriatric assessments could revolutionize the early detection process for MCI, enabling healthcare professionals to swiftly identify and subsequently guide at-risk individuals toward comprehensive cognitive assessments and timely intervention.

This study represents a significant stride forward in early ADRD detection during the MCI stage. A novel, cost-effective tool has been developed that leverages a single depth camera to accurately track twenty-five body joints during both oval and straight walking patterns. The comprehensive approach, which combines advanced signal processing, meticulous statistical analysis, and sophisticated machine learning techniques, facilitates the extraction of certain gait features. These features are then adeptly analyzed by machine learning models, particularly focusing on MCI detection. The findings reveal a notable sensitivity in oval walking, where twenty-seven significant gait features were identified in distinguishing MCI from HC, compared to twenty in straight walking. The RF classifier demonstrated exceptional performance in analyzing oval walking gait measurements, achieving a notable 85.5% accuracy and an 83.9% F-score in detecting MCI. Furthermore, aligning the most important features identified by the RF classifier with those selected through the feature selection methods suggests the potential to refine the model to focus on a smaller yet more effective set of gait characteristics for early ADRD detection. This study underscores the viability of using the depth camera and gait analysis as a powerful tool for early ADRD detection at the MCI stage. Characterized by its affordability, efficiency, simple setup, and non-invasiveness, this method is highly suitable for clinical and non-clinical environments. Its use in routine gait screening could significantly advance early ADRD detection, enabling timely interventions and potentially altering the trajectory of cognitive decline. The research opens new avenues in geriatric care and neurodegenerative disease management, marking a shift in approaching early AD detection and prevention.

Third Experiment: Curve Walking Reveals More Gait Impairments in Older Adults with Mild Cognitive Impairment than Straight Walking.

In this study, the aim is to fill this gap by using a novel system to record the gait of HC and MCI using a non-invasive, low-cost, non-wearable, and the depth camera that can detect and track twenty-five joints of body movement. A comprehensive collection of gait markers was extracted from the recorded signals for both straight and curve walking tests. It was hypothesized that curve walking will be more sensitive to detecting MCI among older adults than straight walking.

This study makes several significant contributions. First, it provides a comprehensive quantitative analysis of gait performance by comparing straight and curved walking using the detailed assessment pro-vided by the depth camera. Second, fifty markers were examined, including macro and micro markers, derived from overall performance and subphases of gait cycles. This comprehensive approach enhances the understanding of gait characteristics. Third, the findings suggest that curve walking offers greater challenges and valuable insights for detecting MCI than straight walking. Moreover, the study demonstrates the feasibility and accessibility of using the depth camera for curve walking assessment, which can complement cognitive assessments and aid in early diagnosis and management of MCI. The impact of this study extends to clinical practice and research, providing improved methods for MCI screening and monitoring in older adults that can be easily replicated with minimal costs and time in the clinic setting.

Participants: A total of fifty-five participants, comprising thirty HC individuals and twenty-five individuals with MCI, were recruited for the study. The individuals with MCI were diagnosed based on a comprehensive evaluation, including brain imaging, electroencephalography (EEG), neuropsychological assessments, and physical examinations. The HC participants underwent a similar evaluation but were without any cognitive impairment. The Persian version of the Montreal Cognitive Assessment (MoCA) and Mini-Mental State Examination (MMSE) were utilized to assess cognitive status. Additionally, the Geriatric Depression Scale (GDS) was employed to measure depression symptoms.

Exclusion criteria for this study included individuals who were diagnosed with moderate to severe depression, were taking medication that could interfere with testing, had undergone surgery such as knee or hip replacement, or had experienced a stroke. All participants could perform the physical tests independently, and none had any visual or hearing impairments that could interfere with the tests.

Gait measurement: A depth camera was used to record the participants' gait while performing two different walking tests: a straight and a curved walking test. Each test covered a distance of ten meters, a recognized standard for reliable gait assessment across populations, including MCI subjects. For each participant, three trials were conducted on both straight and curved paths, with the mean of these trials calculated to ensure robust gait analysis. Median values of gait markers were also used to depict the most consistent walking conditions for individuals. The curve walking test involved walking around an oval path, while the straight walking test was conducted on a straight path.

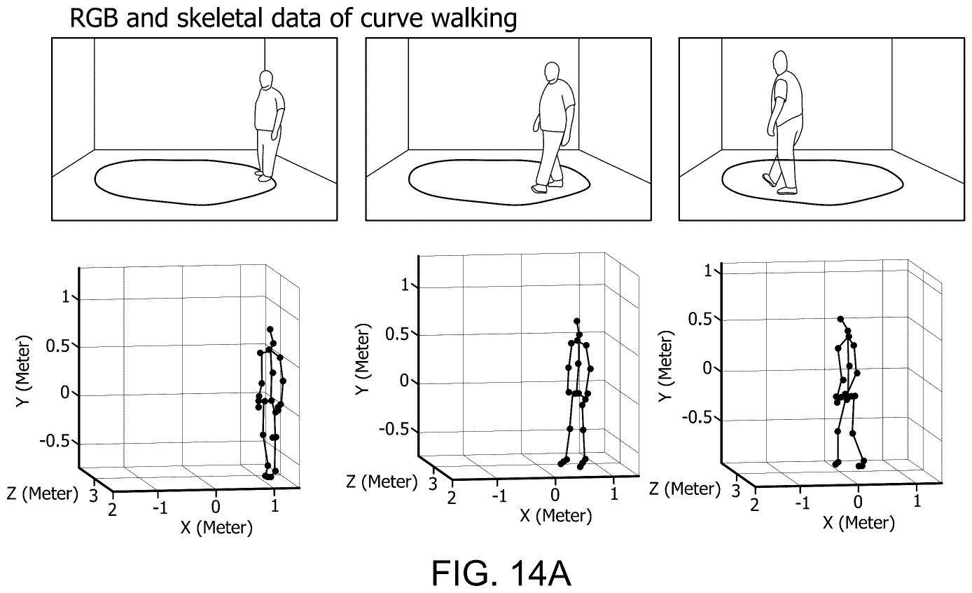

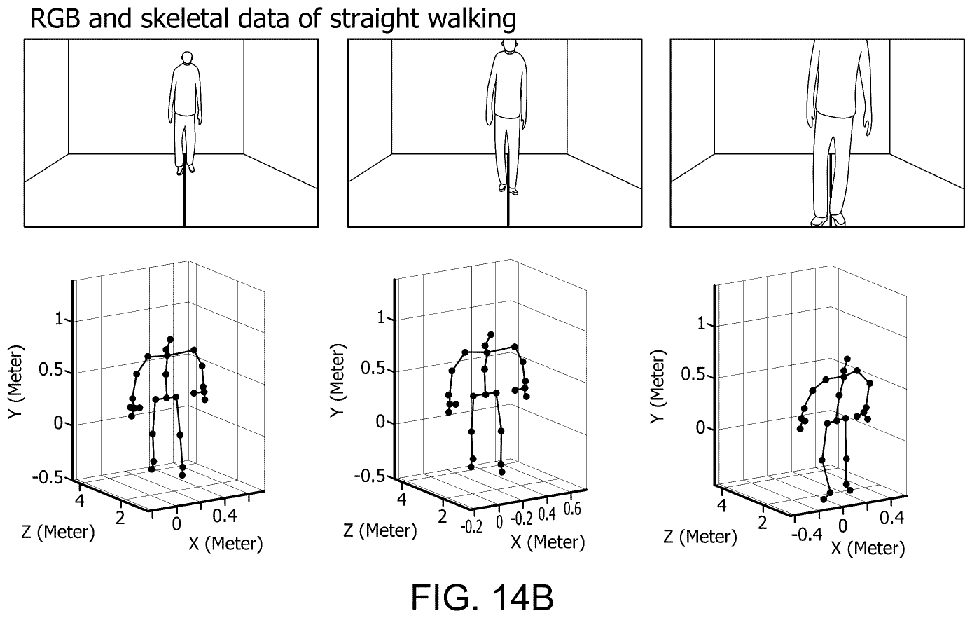

The camera was mounted on a tripod and connected to a laptop. A GUI was used to control the data recording. The camera can detect and track twenty-five joints of the subject's body. FIG. 14 shows recorded samples of straight and curved paths walking beside the skeletal data (locations of body joints). The skeletal data was plotted using the position of the detected and tracked body joints using the camera and GUI.

Data preprocessing: The camera signals were preprocessed before extracting the gait markers. A six-order Butterworth filter with a cutoff frequency of 3 Hz was used to remove noise from the recorded signals of body joints during performing the walking tests.

Gait marker extraction: A comprehensive collection of fifty gait markers for each test separately was extracted from the preprocessed signals. These markers included two macro markers (average velocity and cadence), twenty-four micro temporal markers (duration of feet for various sub-phases of the gait cycle, such as stance, swing, step, and stride phases), eighteen micro spatial markers (location changes of feet for various sub-phases of the gait cycle), and six micro spatiotemporal markers (velocity of feet for various sub-phases of the gait cycle). These markers provided detailed information on the functional performance of the participants during the gait tests.

This study emphasizes spatio-temporal gait markers using the depth camera for better validity. Also, evidence shows the predictive value of spatio-temporal gait markers for kinematic parameters, making them a practical proxy. Clinically, spatio-temporal parameters offer easy interpretation and group comparison.

Statistical analysis: Descriptive statistical analyses were used to compare the clinical and demographic information and the walking markers between the two study groups. The normality of the data was checked using the Shapiro-Wilk test. For normally distributed data, unpaired t-tests were used to compare the gait markers of the two groups, while for non-normally distributed data, Mann-Whitney U tests were used to compare the gait markers of the two groups. For categorical data, such as gender, chi-square tests were used to compare the proportions of the two groups. ANCOVA was used to adjust the extracted gait markers for the confounding variables of BMI, years of education, and GDS scores, which showed significant differences between the two groups. This approach addressed the potential sources of bias and variability in the data analysis. The results were reported as mean±standard deviation (SD) for continuous variables. A p-value of less than 0.05 was considered statistically significant for all analyses (p-value<0.05).

Results: The demographic and clinical characteristics of the participants are shown in TABLE 7. There were no significant differences in age and gender distribution between the two groups. However, the two groups had significant differences in BMI, years of education, and GDS scores. Participants with MCI had a higher BMI, lower levels of education, and higher GDS scores than HCs. The extracted gait markers were adjusted for these confounding factors in the analysis, as they may impact physical test performance. As expected, the MCI group had lower scores on the MoCA and MMSE tests than the HC group.

TABLE 7
Demographic and clinical information

| | HC (n = 30) | MCI (n = 25) | |
|---|---|---|---|
| Characteristic | mean ± SD | mean ± SD | P |
| Age (y) | 68.33 SD 2.15 | 69.76 ± 6.45 | 0.091 |
| Female gender, n (%) | 18 (60) | 19 (76) | 0.216 |
| BMI (kg/m²) | 24.51 ± 2.67 | 26.67 ± 2.62 | <0.001* |
| Years of education | 13.53 ± 3.05 | 11.56 ± 3.00 | 0.008* |
| MMSE | 28.50 ± 1.17 | 25.60 ± 1.29 | <0.001* |
| MoCA | 27.13 ± 2.05 | 22.76 ± 1.69 | <0.001* |
| GDS | 1.43 ± 1.33 | 3.52 ± 1.29 | <0.001* |

Mean ± Standard deviation was shown. n, number of subjects; HC, healthy cognitive control group; MCI, mild cognitive impairment; AD, Alzheimer's disease; BMI, body mass index; MMSE, Mini-Mental State Examination (maximum score, 30); MoCA, Montreal Cognitive Assessment (maximum score, 30); GDS, Geriatric Depression Scale (maximum score, 15);
*Significant difference at p-value < 0.05.

Gait performance was analyzed in both conditions to investigate the sensitivity of curve walking com-pared to straight walking for detecting MCI among older adults. Fifty gait markers were extracted from the recorded signals for each condition, controlling for confounding factors such as BMI, years of education, and GDS scores. These markers were classified into four categories: macro, micro temporal, micro spatial, and micro spatiotemporal. TABLE 8 presents the mean, standard deviation, and p-value of each marker for both groups and conditions.

TABLE 8
Extracted markers from straight and curve walking and their comparison results

| Feature | | Straight Walking | | | Curve Walking | | |
|---|---|---|---|---|---|---|---|
| Type | Name | HC | MCI | P | HC | MCI | P |
| Macro | Velocity (cm/s) | 50.13 ± 27.92 | 47.39 ± 9.80 | 0.780 | 45.90 ± 8.89 | 39.27 ± 10.09 | 0.007* |
| | Cadence (steps/min) | 68.22 ± 10.55 | 68.90 ± 9.70 | 0.247 | 62.02 ± 9.73 | 55.00 ± 11.67 | 0.018* |
| Micro | Stance T. me (s) | 0.78 ± 0.24 | 0.83 ± 0.34 | 0.716 | 0.57 ± 0.15 | 0.64 ± 0.22 | 0.138 |
| Temporal | Stance T. var (%) | 18.15 ± 20.91 | 23.48 ± 26.30 | 0.993 | 49.91 ± 18.75 | 73.08 ± 20.27 | <0.001* |
| | Stance T. med (s) | 0.78 ± 0.24 | 0.84 ± 0.33 | 0.729 | 0.53 ± 0.17 | 0.52 ± 0.23 | 0.384 |
| | Swing T. me (s) | 0.71 ± 0.15 | 0.72 ± 0.16 | 0.841 | 0.54 ± 0.04 | 0.52 ± 0.07 | 0.211 |
| | Swing T. var(s) | 12.41 ± 16.09 | 19.83 ± 13.89 | 0.053 | 26.10 ± 8.00 | 32.89 ± 11.12 | 0.018* |
| | Swing T. med (s) | 0.69 ± 0.15 | 0.72 ± 0.16 | 0.742 | 0.51 ± 0.05 | 0.48 ± 0.06 | 0.001* |
| | DS T. me (s) | 0.21 ± 0.11 | 0.25 ± 0.14 | 0.323 | 0.21 ± 0.04 | 0.31 ± 0.09 | <0.001* |
| | DS T. var (%) | 69.63 ± 38.82 | 80.65 ± 26.45 | 0.407 | 80.16 ± 22.57 | 91.91 ± 21.54 | 0.072 |
| | DS T. med (s) | 0.13 ± 0.08 | 0.17 ± 0.10 | 0.080 | 0.17 ± 0.05 | 0.23 ± 0.05 | <0.001* |
| | SS T. me (s) | 0.53 ± 0.16 | 0.52 ± 0.15 | 0.498 | 0.40 ± 0.08 | 0.35 ± 0.09 | 0.014* |
| | SS T. var (%) | 37.30 ± 23.06 | 45.14 ± 25.72 | 0.196 | 49.00 ± 13.07 | 48.56 ± 11.29 | 0.896 |
| | SS T. med (s) | 0.57 ± 0.18 | 0.55 ± 0.18 | 0.496 | 0.36 ± 0.09 | 0.34 ± 0.10 | 0.158 |
| | Step T. me (s) | 0.77 ± 0.13 | 0.78 ± 0.11 | 0.331 | 0.88 ± 0.13 | 1.05 ± 0.27 | 0.012* |
| | Step T. var (%) | 18.05 ± 16.60 | 27.13 ± 11.29 | 0.042* | 34.08 ± 17.07 | 40.51 ± 10.50 | 0.017* |
| | Step T. med (s) | 0.76 ± 0.09 | 0.77 ± 0.10 | 0.606 | 0.83 ± 0.09 | 0.94 ± 0.20 | 0.013* |
| | Step T. sym me | 0.64 ± 0.08 | 0.64 ± 0.06 | 0.680 | 0.83 ± 0.03 | 0.84 ± 0.03 | 0.207 |
| | Step T. sym var (%) | 24.22 ± 20.21 | 34.77 ± 14.35 | 0.035* | 14.64 ± 5.93 | 14.45 ± 5.27 | 0.899 |
| | Step T. sym med | 0.68 ± 0.08 | 0.70 ± 0.08 | 0.452 | 0.87 ± 0.03 | 0.88 ± 0.03 | 0.019* |
| | Stride T. me (s) | 1.55 ± 0.30 | 1.54 ± 0.22 | 0.254 | 1.77 ± 0.26 | 2.08 ± 0.53 | 0.016* |
| | Stride T. var (%) | 13.66 ± 14.58 | 19.61 ± 9.54 | 0.049* | 26.12 ± 12.36 | 29.41 ± 10.98 | 0.261 |
| | Stride t. med (s) | 1.57 ± 0.30 | 1.58 ± 0.25 | 0.496 | 1.67 ± 0.18 | 1.92 ± 0.42 | 0.024* |
| | Stride t. reg me | 0.94 ± 0.06 | 0.77 ± 0.12 | <0.001* | 0.76 ± 0.09 | 0.74 ± 0.09 | 0.414 |
| | Stride T. reg var (%) | 0.37 ± 2.35 | 5.73 ± 10.14 | 0.118 | 21.75 ± 14.39 | 18.65 ± 9.16 | 0.729 |
| | Stride T. reg med | 0.94 ± 0.06 | 0.77 ± 0.12 | <0.001* | 0.75 ± 0.11 | 0.74 ± 0.11 | 0.676 |
| Micro | Step L. me (cm) | 35.75 ± 5.53 | 31.06 ± 6.74 | 0.003* | 37.58 ± 4.97 | 32.91 ± 3.46 | <0.001* |
| Spatial | Step L., var (%) | 21.21 ± 18.29 | 32.65 ± 19.51 | 0.016* | 37.01 ± 9.97 | 41.84 ± 9.57 | 0.075 |
| | Step L. med (cm) | 37.63 ± 5.12 | 30.99 ± 9.19 | <0.001* | 40.16 ± 5.76 | 34.22 ± 4.90 | <0.001* |
| | Step L. sym me | 0.80 ± 0.17 | 0.74 ± 0.19 | 0.106 | 0.70 ± 0.10 | 0.66 ± 0.11 | 0.114 |
| | Step L. sym var (%) | 22.16 ± 31.41 | 21.88 ± 15.86 | 0.174 | 34.87 ± 12.43 | 38.87 ± 13.25 | 0.255 |
| | Step L. sym med | 0.83 ± 0.18 | 0.75 ± 0.19 | 0.025* | 0.75 ± 0.13 | 0.67 ± 0.16 | 0.030* |
| | Step W. me(m) | 0.12 ± 0.03 | 0.13 ± 0.03 | 0.919 | 0.21 ± 0.04 | 0.17 ± 0.03 | 0.002* |
| | Step W. var (%) | 30.91 ± 12.54 | 23.10 ± 11.13 | 0.013* | 56.82 ± 12.33 | 67.09 ± 13.87 | 0.011* |
| | Step W. med (m) | 0.13 ± 0.03 | 0.13 ± 0.03 | 0.912 | 0.21 ± 0.06 | 0.16 ± 0.04 | 0.022* |
| | Step H. me (m) | 0.11 ± 0.04 | 0.08 ± 0.04 | 0.003* | 0.12 ± 0.02 | 0.13 ± 0.02 | 0.069 |
| | Step H. var (%) | 28.56 ± 21.18 | 39.24 ± 14.15 | 0.012* | 38.65 ± 14.60 | 46.29 ± 11.20 | 0.004* |
| | Step H. med (m) | 0.11 ± 0.04 | 0.08 ± 0.05 | 0.020* | 0.11 ± 0.02 | 0.12 ± 0.02 | 0.114 |
| | Stride L. me (cm) | 74.02 ± 11.16 | 63.62 ± 14.92 | 0.006* | 75.84 ± 10.44 | 65.91 ± 6.83 | <0.001* |
| | Stride L. var (%) | 11.82 ± 14.75 | 21.45 ± 13.08 | 0.013* | 27.84 ± 11.09 | 30.83 ± 12.33 | 0.393 |
| | Stride L. med (cm) | 74.36 ± 12.32 | 63.88 ± 15.35 | 0.002* | 76.46 ± 11.61 | 65.10 ± 9.50 | <0.001* |
| | Stride L. reg me | 0.96 ± 0.06 | 0.79 ± 0.13 | <0.001* | 0.68 ± 0.12 | 0.70 ± 0.12 | 0.657 |
| | Stride L. reg var (%) | 2.44 ± 2.72 | 6.60 ± 14.42 | 1.00 | 23.37 ± 15.17 | 24.66 ± 14.71 | 0.666 |
| | Stride L. reg med | 0.96 ± 0.06 | 0.79 ± 0.13 | <0.001* | 0.68 ± 0.13 | 0.71 ± 0.15 | 0.369 |
| Micro | Step V. me (cm/s) | 47.26 ± 7.04 | 45.62 ± 13.01 | 0.574 | 46.41 ± 8.05 | 37.17 ± 8.08 | <0.001* |
| Spatiotemporal | Step V. var (%) | 30.75 ± 19.45 | 52.01 ± 21.87 | <0.001* | 47.66 ± 9.92 | 53.47 ± 14.37 | 0.083 |
| | Step V. med (cm/s) | 47.65 ± 8.28 | 41.67 ± 12.88 | 0.139 | 46.88 ± 9.48 | 35.08 ± 9.92 | <0.001* |
| | Step V. me (cm/s) | 48.08 ± 7.22 | 43.65 ± 12.81 | 0.177 | 45.51 ± 7.91 | 35.47 ± 8.51 | <0.001* |
| | Stride V. var (%) | 14.47 ± 14.88 | 28.80 ± 13.21 | 0.001* | 37.78 ± 11.52 | 38.89 ± 10.05 | 0.704 |
| | Stride V. med (cm/s) | 48.13 ± 7.97 | 43.16 ± 12.73 | 0.165 | 46.35 ± 8.48 | 34.15 ± 10.06 | <0.001* |

Data are shown as mean ± standard deviation; p-value is reported for comparison between two study groups;
*Significant difference at p-value < 0.05.
HC, healthy control without cognitive impairment; MCI, mild cognitive impairment; T, time; L, length; W, width; H, height; V, velocity; me, mean; var, variability; sym, symmetry; reg, regularity; DS, double support; SS, single support.

Curve Walking (CW) test: In the CW test, significant differences were observed between the HC and MCI groups in twenty-seven out of the fifty extracted markers. Among these markers, two were macro markers, namely average velocity, and cadence, while the remaining twenty-five were micro markers. The micro markers comprised twelve micro temporal markers, nine micro spatial markers, and four micro spatiotemporal markers.

Straight Walking (SW) test: Conversely, the SW test revealed significant differences in twenty out of the fifty extracted markers. None of these markers were macro markers; instead, they consisted of five micro temporal markers, thirteen micro spatial markers, and two micro spatiotemporal markers.

Comparison of CW and SW markers: Comparing the markers with significant differences between CW and SW, it was found that nine-teen out of the twenty-seven significant markers in CW were unique and did not show significant differences in SW. On the other hand, twelve markers in SW were unique and exhibited significant differences only in SW. Additionally, eight markers were common in both CW and SW conditions, showing significant differences in both tasks.

Unique significant markers in CW: In CW, the unique significant markers included two macro markers (velocity and cadence), eleven temporal markers (mean and median of double support time (DST), single support time (SST), step time, and stride time, variability of stance and swing time, as well as symmetry of step time), two spatial micro markers (mean and median of step width), and four spatiotemporal markers (mean and median of step and stride velocity).

Unique significant markers in SW: For SW, the unique significant markers comprised four micro temporal markers (variability of stride time and step time symmetry, as well as mean and median of stride time regularity), six micro spatial markers (mean and median of step height, and stride length regularity, as well as the variability of step and stride length), and two micro spatiotemporal markers (variability of step and stride velocity).

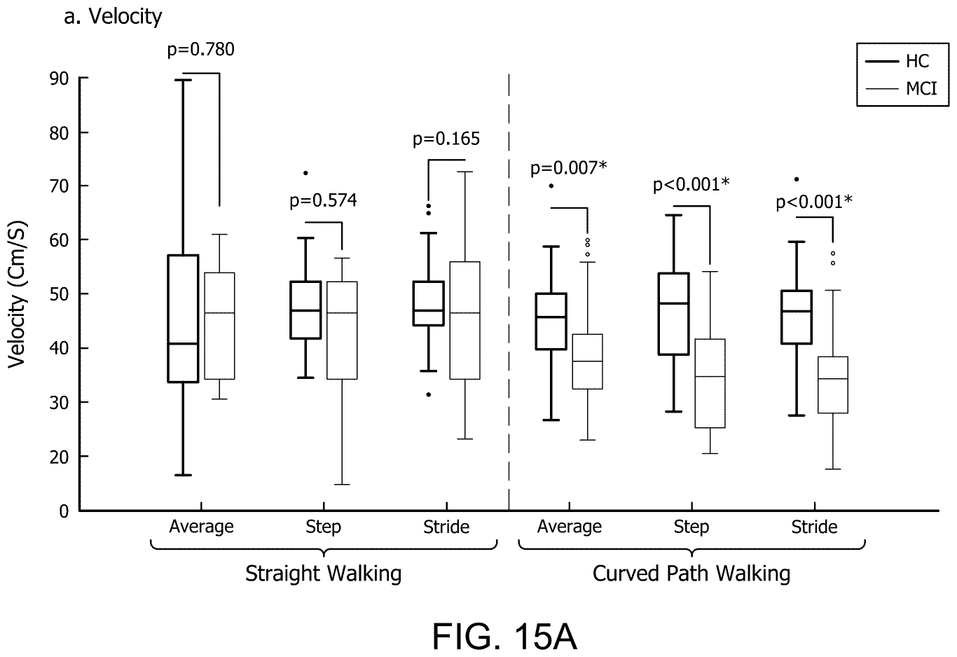

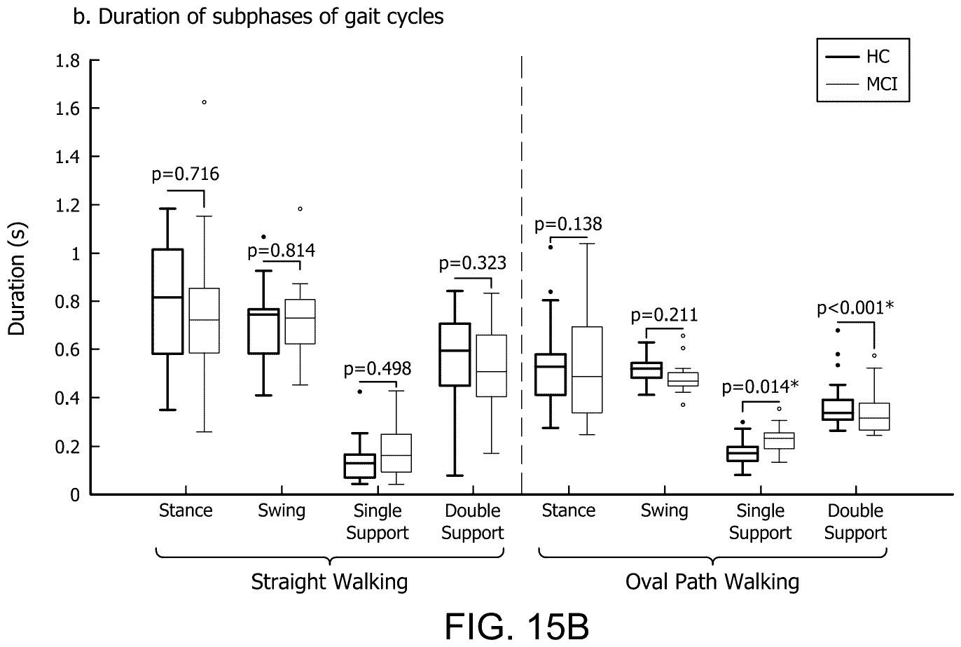

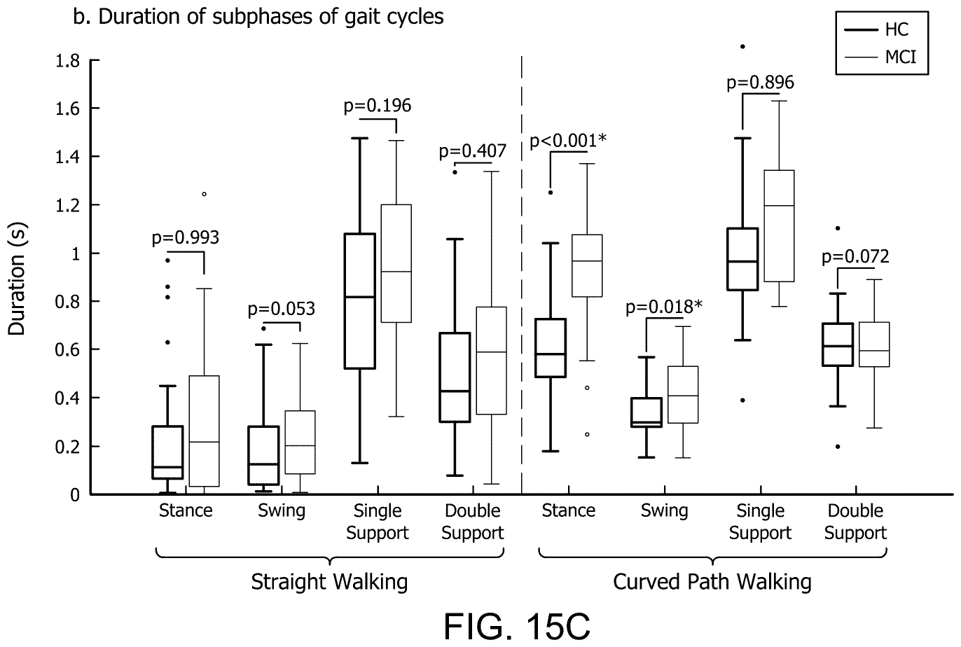

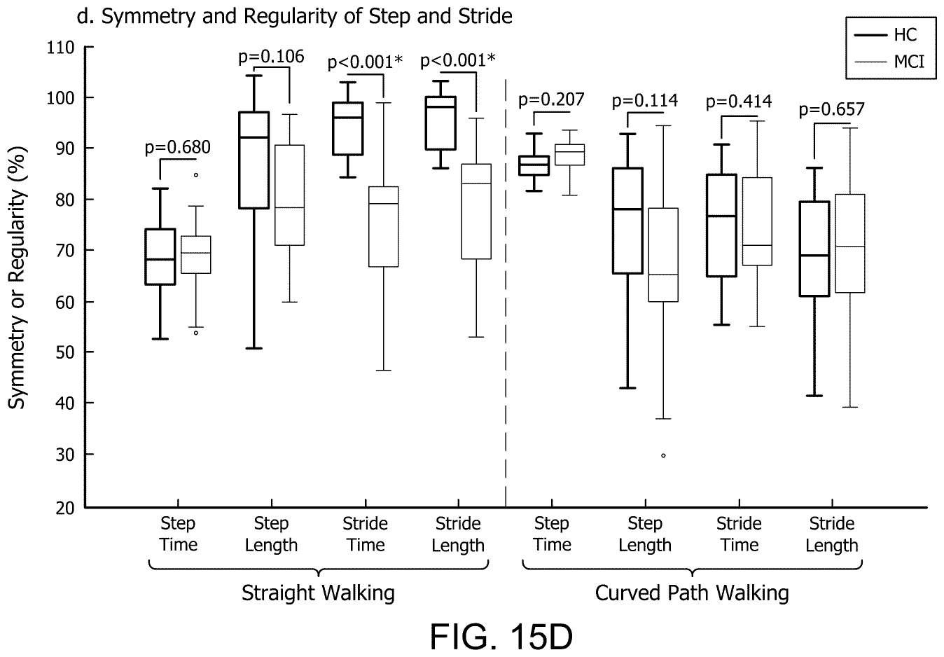

Common significant markers in CW and SW: The common markers between CW and SW conditions consisted of one temporal marker (step time variability) and seven micro spatial markers (mean of step and stride length, median of step length, step length symmetry, and stride length regularity, as well as variability of step length and step height). FIG. 15 provides a visual representation of the performance comparison between the HC and MCI subjects in CW and SW conditions. In straight walking, older adults with MCI exhibited lower average velocity (47.39±9.80 mean±SD), step velocity (45.62±13.01), and stride velocity (43.65±12.81) compared to HC (50.13±27.92, 47.26±7.04, and 48.08±7.22, respectively). In CW, average velocity, step velocity, and stride velocity significantly decreased among MCIs to 39.27±10.09, 37.17±8.08, and 35.47±8.51 (cm/s), respectively. The values listed in TABLE 2 have been adjusted for BMI.

Temporal and spatial markers: The changes in mean and median values of micro temporal and spatial markers were not consistently in the same direction. Double support, step time, and stride time increased among study groups, especially in MCIs, while stance, swing, and single support time decreased in CW. For example, step time increased to 1.05±0.27 seconds (s) for MCIs and 0.88±0.13 (s) for HCs in CW, while these markers were only 0.78±0.11 (s) and 0.77±0.13 (s) for both groups in the SW condition (FIG. 15B). In comparison, stance time changed from 0.83±0.34 (s) to 0.64±0.22 (s) for MCIs when transitioning from straight to curve walking (FIG. 15B).

Variability of gait cycle subphases: The variability of subphases of the gait cycle differed between SW and CW. In CW, the variability of subphases such as stance, swing, double and single support, step, and stride as micro temporal features increased, and these changes were more pronounced among MCIs. FIG. 15C illustrates the changes in the variability of different sub-phases of gait cycles between the two study groups across different walking tests. For instance, the variability of stance and swing time increased from 23.48±26.30% and 19.83±13.89% to 73.08±20.27% and 32.89±11.12% for MCIs, and from 18.15±20.91% and 12.41±16.09% to 49.91±18.75% and 26.10±8.00% for HCs when transitioning from straight to curve walking. Similar changes were observed for the variability of micro spatial markers, where the variability of step width and height increased from 23.10±11.13% and 39.24±14.15% to 67.09±13.87% and 46.29±11.20% for MCIs, while these markers increased from 30.91±12.54% and 28.56±12.18% to 56.82±12.33% and 38.65±14.60% for HCs with the transition from straight walking to curve walking.

Symmetry and regularity markers: A total of twelve markers related to symmetry and regularity were extracted from step and stride, including their mean, median, and variability. Among these markers, mean and median values generally decreased from SW to CW for both study groups, except for the mean and median of step time. However, for variability, the variability of step and stride time symmetry decreased to 14.45±5.27% and 18.65±9.16% for MCIs, and to 14.64±5.93% and 21.75±14.39% for HCs, respectively. In contrast, the variability of step and stride length symmetry and regularity increased when transitioning from SW to CW (see FIG. 15D).

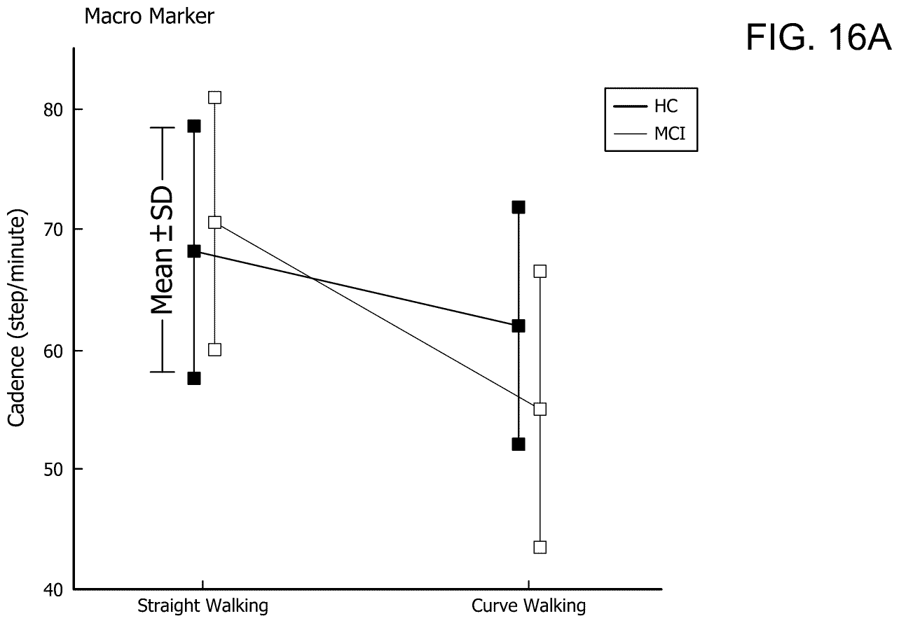

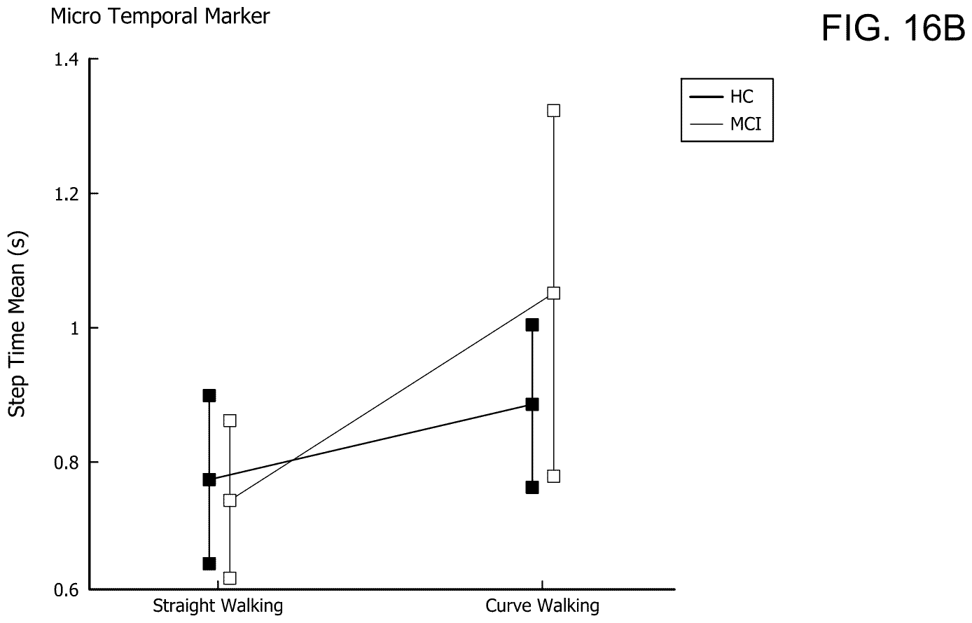

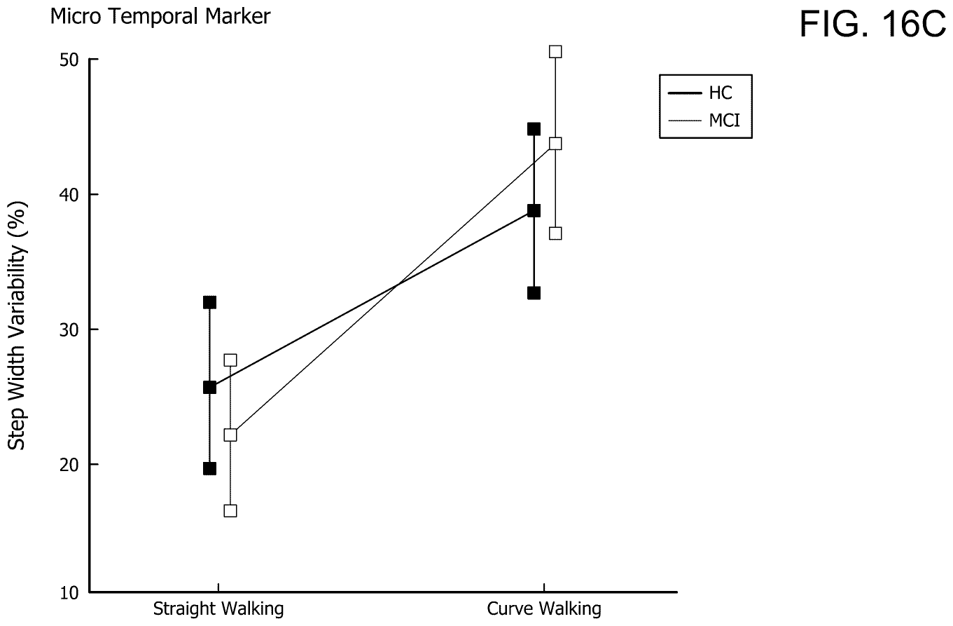

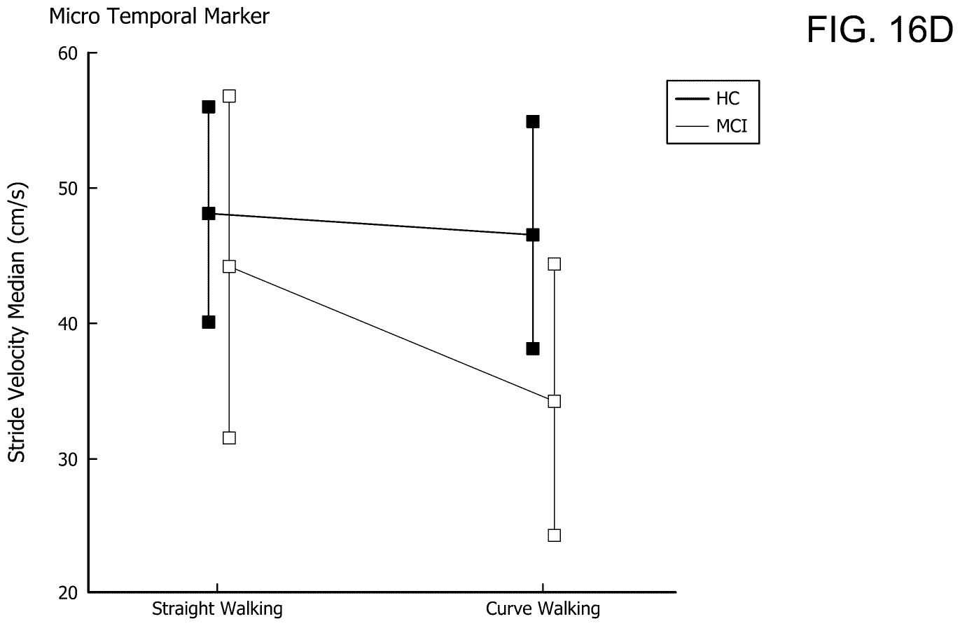

Effect of curve path on subject's walking: To examine the impact of changing the walking test from straight walking to curve walking on MCI and HC participants, the costs of the walking test were calculated. The results showed that the gait of MCI older adults was affected more than HCs when the walking test changed from SW to CW. The costs of thirty-one out of fifty gait markers (62%) were greater for MCI than HCs when the walking tests changed from SW to CW, and thirteen marker costs showed significant differences between the two study groups. FIG. 16 shows the changes for different types of gait markers were more significantly affected when the walking test changed from SW to CW among HC and MCI participants (these markers had lower p-values for the cost comparison of MCI versus HCs). The cadence of the MCI group decreased from 68.90±9.70 (steps/minute) to 55.00±11.67 (steps/minute), while it only changed from 68.22±10.55 (steps/minute) to 62.02±9.73 (steps/minute) for HCs (see FIG. 16A).

For micro temporal gait markers of step time mean, it increased from 0.78±0.11 (s) to 1.05±0.27 (s) for MCI participants versus 0.77±0.13 (s) to 0.88±0.13 (s) for HCs (see FIG. 16B). Also, the micro spatial marker of the step width variability (see FIG. 16C) significantly rose among MCIs (from 23.10±11.13% to 67.09±13.87%). The micro spatiotemporal marker of stride velocity median had the highest changes among spatiotemporal markers, costing 22.27%. The stride velocity median decreased from 43.16±12.73 to 34.15±10.06 (cm/s) for MCIs versus 48.13±7.97 to 46.35±8.48 (cm/s) for HCs (see FIG. 16D).

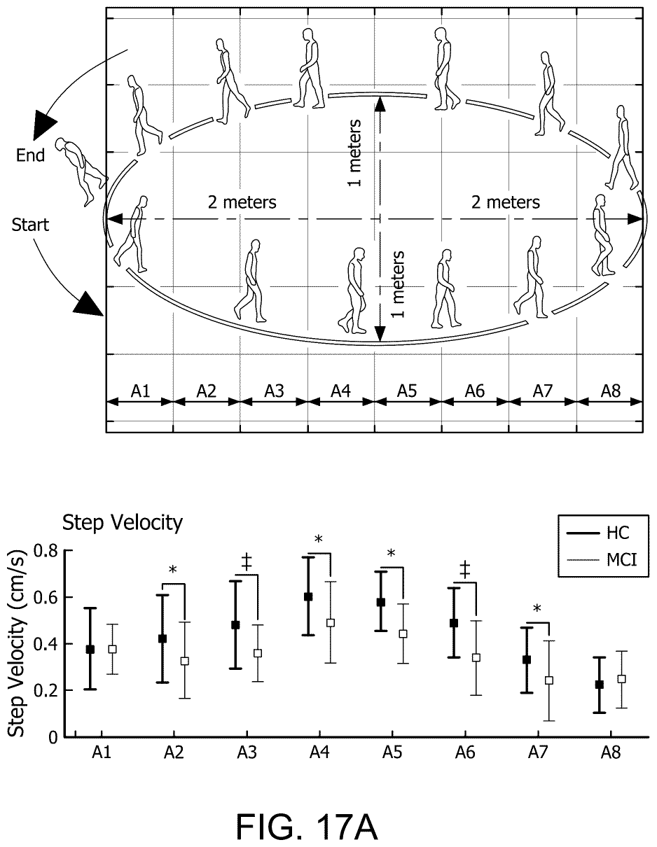

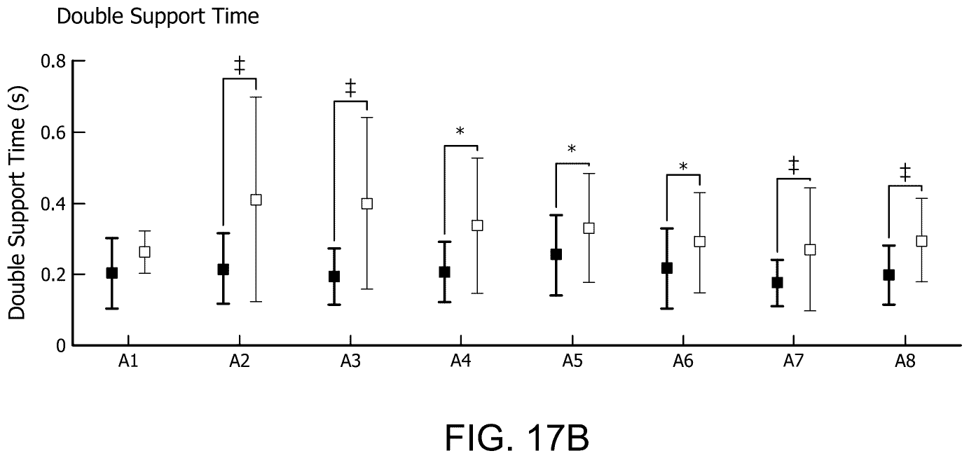

The performance of participants was studied for different areas of the oval path, referred to as different sections of curved path walking. Participants' performance for other gait markers changed in different areas. FIG. 17 shows the curve path with eight different areas (A1 to A8 presents the center of each area) beside the mean±SD of two gait markers for MCI and HC subjects. As seen in FIG. 17A, the step velocity of walking was lower among MCI than HCs in most areas, and significant differences were observed between the two study groups.

In the A1 and A2 areas, during the starting or ending the walking and turning on the right corner of the path, no significant difference was observed between the two study groups, and both study groups had approximately the same step velocity. In comparison, there were significant differences in step velocity for A2 to A7 areas, and the differences were even much more significant in A3 and A6 areas, where the sub-jects started to change their walking from a curve pattern to a straight pattern or vice versa. In addition, when HC participants were in the middle of the walking path (A4 to A5), their step velocity increased as the path was approximately straight, and this rise was less among MCIs.

The double support time was examined as a micro temporal marker of gait that shows the balance of people during walking for various areas of the curve path (see FIG. 17B). Older adults with MCI needed more double support time than HCs. The double support time increased among MCIs in different areas significantly, while it changed slightly among HCs in various areas of the curve path. MCI participants spent considerably more time for double support of the gait cycle when they needed to change their walking directions (A2, A3, and A7 areas of the curve path) and during turning on the right corner of the curve path (A8 area of the curve path). During the transition sections A2 and A3, from curved to straight paths, MCI participants demonstrated a wider range of DST values than those in the HC group. This increased variability in the MCI group may be due to factors such as adjusting walking speed for path shape changes, balance maintenance, and decision-making during directional shifts.

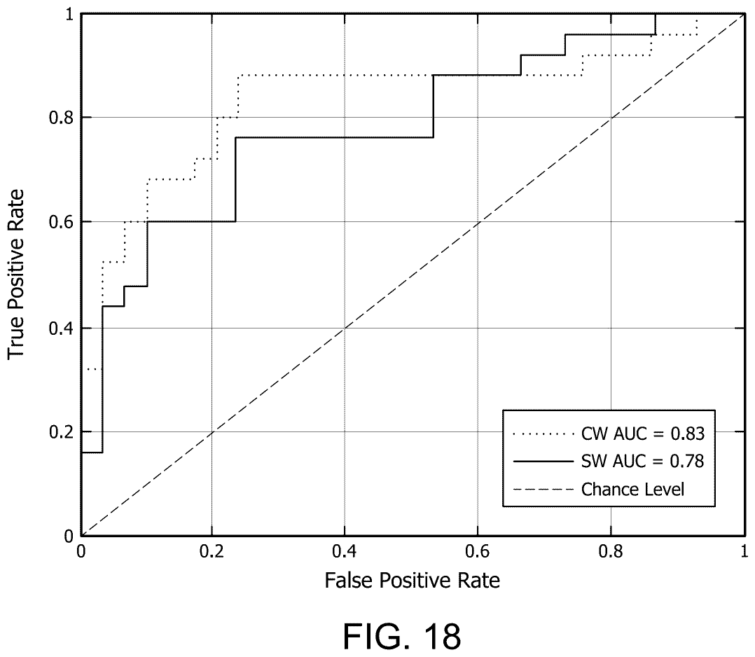

To assess the potential of curved path walking as a diagnostic tool for MCI, a receiver-operating characteristic (ROC) analysis was conducted. This analysis utilized significant gait markers to determine their effectiveness in differentiating MCI individuals from the normal elderly population in both curved and straight walking scenarios. FIG. 18 displays the ROC curves for MCI detection using these significant gait markers. The results showed that the area under the curve (AUC) for curved path walking (0.83) was significantly higher than for straight path walking (0.78), highlighting curved path walking's enhanced ability to identify MCI.

Discussion: MCI is detected due to its potential association with AD and other forms of dementia. Early identification, particularly in MCI related to AD, offers an opportunity for timely intervention with amyloid-P monoclonal antibodies. Early detection also facilitates lifestyle adjustments, cognitive rehabilitation, and proactive planning, potentially delaying cognitive decline progression and improving the overall quality of life. Swift medical attention is imperative upon observing cognitive changes or MCI symptoms.

Clinical methods for assessing cognitive impairment, like cognitive tests and brain imaging, have limitations in accuracy, cost, and accessibility. Gait analysis emerges as a valuable, non-invasive complement to these evaluations, providing certain insights into the functional abilities of older adults. The study, however, ventures into the realm of curved path walking—a more natural yet complex activity that demands trajectory prediction and enhanced postural control. This approach is particularly effective in detecting subtle gait impairments that are often prevalent in the early stages of cognitive decline, such as MCI. The gait performance of older adults was compared with and without MCI under both straight and curved walking conditions, utilizing the depth camera, an affordable and non-intrusive method for gait signal capture. The study included fifty-five older adults, twenty-five with MCI and thirty HC, who performed single-task walking tests on straight and oval paths. The signals from twenty-five body joints were processed to extract fifty gait markers for each test, and these markers were compared between the two groups using descriptive statistical analyses.

This study revealed that older adults with MCI exhibited reduced walking performance compared to HC. Notably, the differences in gait markers were more pronounced during curved path walking as opposed to straight walking, suggesting a heightened sensitivity of curved path conditions in detecting gait impairments associated with MCI. As illustrated in FIG. 15, the MCI group exhibited a notably lower average velocity and cadence during curve walking, coupled with higher variability across most micro-gait markers. Additionally, this group demonstrated diminished symmetry and regularity in both step and stride lengths for curved walking. Interestingly, certain gait markers, such as average velocity, did not show significant deviations during straight walking. The differential impact of curved walking on the MCI group compared to the HC group is further highlighted by the cost of the gait markers, as depicted in FIG. 16. Specifically, the MCI group faced more challenges during curved walking, necessitating extended double support time in various areas, especially while changing direction. This was accompanied by a reduction in step velocity in these areas, as illustrated by FIG. 17. Four individuals with MCI, during curve walking, underwent an elevated cognitive load. This heightened cognitive demand results in extended adjustment times for directional changes and balance control. Furthermore, apprehensions regarding potential falls compromised spatial awareness, and intricate motor planning may lead MCI subjects to adopt a more cautious approach during curved walking. Taken together, the findings underscore the heightened sensitivity of gait markers in response to the challenges introduced by curve walking. These markers offer promising potential as early indicators of cognitive impairment.

The comprehensive analysis of curved walking could provide richer insights into MCI detection than a sole emphasis on macro markers or straight walking. Expanding the scope, the results align with research that has evaluated curved walking across diverse groups. Specifically, the challenges of Parkinson's disease subjects became evident when analyzing their straight walking and a 90° left turn. Through gait analysis and motion capture, these subjects displayed decreased walking speed, more steps, and reduced head-trunk coordination during turns. These curved walking challenges are believed to stem from the compromised high-level neural systems commonly found in Parkinson's disease.

This study underscores the potential of curved path walking as a valuable tool for early MCI detection, a step in enhancing subject care and intervention strategies. Curved path walking may be more sensitive for MCI detection, indicating that integrating cognitive tasks with curved walking could improve diagnostic accuracy.

While this study emphasized MCI and HC participants, the present solution is not limited in this regard. The present solution may: incorporate dual-task walking tests or biomarker verification for MCI diagnoses; use a standardized protocol for curve walking testing (e.g., defining optimal length, shape, and trial count); perform concurrent gait-EEG assessments; and/or integrate dual-task walking tests. Through these comprehensive approaches, the aim is to foster advancements in the field, refine assessment techniques, and deepen the understanding of gait discrepancies in MCI and associated cognitive disorders.

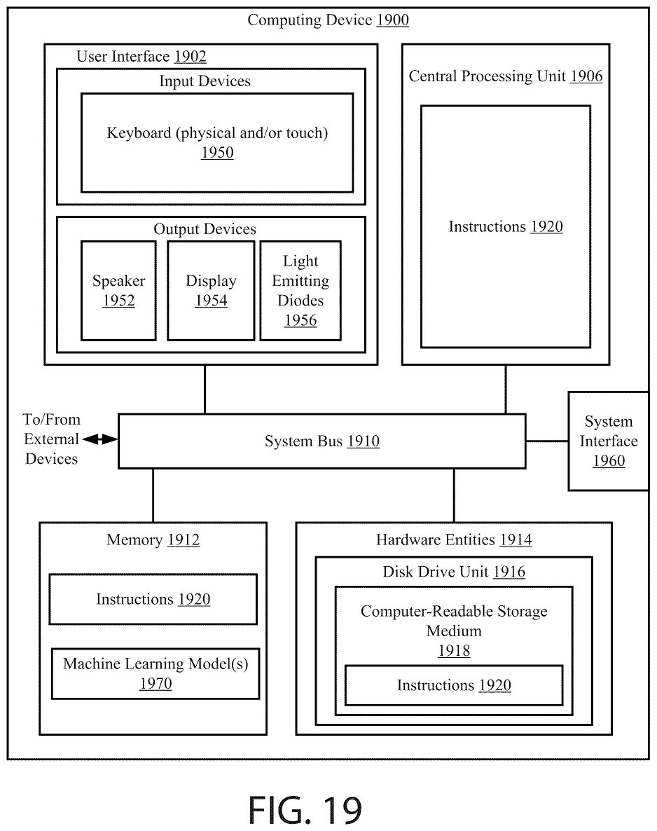

Referring now to FIG. 19, there is shown an illustrative architecture for a computing device 1900. Device(s) 102, 104, 112, 114 of FIG. 11 is(are) the same as or similar to computing device 1900. As such, the discussion of computing device 1900 is sufficient for understanding the devices 102, 104, 112, 114 of FIG. 1.

Computing device 1900 may include more or less components than those shown in FIG. 19. However, the components shown are sufficient to disclose an illustrative solution implementing the present solution. The hardware architecture of FIG. 19 represents one implementation of a representative computing device configured to receive information, process the receive information, transmit information and/or control operations of an aerial vehicle, as described herein. As such, the computing device 1900 of FIG. 19 implements at least a portion of the method(s) described herein.

Some or all components of the computing device 1900 can be implemented as hardware, software and/or a combination of hardware and software. The hardware includes, but is not limited to, one or more electronic circuits. The electronic circuits can include, but are not limited to, passive components (e.g., resistors and capacitors) and/or active components (e.g., amplifiers and/or microprocessors). The passive and/or active components can be adapted to, arranged to and/or programmed to perform one or more of the methodologies, procedures, or functions described herein.

As shown in FIG. 19, the computing device 1900 comprises a user interface 1902, a central processing unit (CPU) 1906, a system bus 1910, a memory 1912 connected to and accessible by other portions of computing device 1900 through system bus 1910, a system interface 1960, and hardware entities 1914 connected to system bus 1910. The user interface can include input devices and output devices, which facilitate user-software interactions for controlling operations of the computing device 1900. The input devices include, but are not limited to, a physical and/or touch keyboard 1950. The input devices can be connected to the computing device 1900 via a wired or wireless connection (e.g., a Bluetooth® connection). The output devices include, but are not limited to, a speaker 1952, a display 1954, and/or light emitting diodes 1956. System interface 1960 is configured to facilitate wired or wireless communications to and from external devices (e.g., network nodes such as access points, etc.).

At least some of the hardware entities 1914 perform actions involving access to and use of memory 1912, which can be a Random Access Memory (RAM), a disk drive, flash memory, and/or another hardware device that is capable of storing instructions and data. Hardware entities 1914 can include a disk drive unit 1916 comprising a computer-readable storage medium 1918 on which is stored one or more sets of instructions 1920 (e.g., software code) configured to implement one or more of the methodologies, procedures, or functions described herein. The instructions 1920 can also reside, completely or at least partially, within the memory 1912 and/or within the CPU 1906 during execution thereof by the computing device 1900. The memory 1912 and the CPU 1906 also can constitute machine-readable media. The term “machine-readable media”, as used here, refers to a single medium or multiple media (e.g., a centralized or distributed database, and/or associated caches and servers) that store the one or more sets of instructions 1920. The term “machine-readable media”, as used here, also refers to any medium that is capable of storing, encoding or carrying a set of instructions 1920 for execution by the computing device 1900 and that cause the computing device 1900 to perform any one or more of the methodologies of the present disclosure.

In view of the forgoing, the present solution generally concerns implementing systems and methods for comprising: obtaining, by at least one processor, video data depicting a subject walking along a predetermined path; processing the video data to generate, by the at least one processor, a data object for the subject; determining, by the at least one processor, a plurality of gait features and balance features for the subject from the data object; determining, by the at least one processor and using a machine learning model, whether the subject has cognitive impairment (e.g., Alzheimer's disease), based, at least in part on analysis of the determined plurality of gait features and balance features; and/or determining, by the at least one processor, a cognitive load associated with the subject walking along the predetermined path (wherein the determined cognitive load is used to determine whether the subject has the cognitive impairment). The predetermined path may comprise a curved path, a figure-of-eight path, and/or a straight path. The plurality of gait features and balance features may comprise micro features and macro features. Micro features reflect more detailed aspects of the gait cycle. Micro features may be classified into the following: micro temporal features (e.g., stride time, step time, stance time, swing time, single and double support time), micro spatial features (e.g., stride length, step length, step count, and/or a stride count), and micro spatiotemporal features (e.g., step velocity, stride velocity, foot position change during a gait subphase, walking duration, and/or average velocity). Macro features capture overall performance and include average gait velocity and cadence. In this regard, macro features can include, but are not limited to, average velocity, cadence, and/or gait cycle subphase duration.

The video data processing may comprise: transforming the video data from three-dimensional video data to two-dimensional video data or transforming the video data from two-dimensional video data to three-dimensional video data; and/or applying a two-dimensional (2D) human pose estimation technique. The video data may comprise three-dimensional video data obtained via a depth camera. The video data may be obtained via a mobile device camera.

The data object may be generated by: detecting body joints within the video data; preprocessing or filtering signals from the detected body joints to remove noise signals; and extracting relevant features corresponding with at least some of the detected joints.

The machine learning model may comprise SVM classifier or deep learning model. The machine learning model may be trained by: identifying, by the at least one processor, one or more significant gait features or balance features between at least one AD data set and at least one healthy control (HC) data set, and adjusting, by the at least one processor, the one or more significant gait features or balance features using ANCOVA technique. The adjusting may comprise adjusting for age, weight, and/or education for each subject in the at least one AD data set and/or the at least one HC data set.

The methods may further comprise obtaining, by the at least one processor, cognitive task data for the subject, while the subject is walking along the predetermined path and performing at least one cognitive task, wherein determining whether the subject has cognitive impairment comprises analyzing the cognitive task data in conjunction with the video data. The cognitive task data may be obtained via one or more sensors positioned in proximity to the subject's body.

The present solution also concerns a system comprising: a mobile device including a processor having an application (App) executing thereon; and at least one computing device in electronic communication with the mobile device, the at least one computing device comprising a processor, and a memory having instructions thereon, wherein the instructions when executed by the processor, cause the processor to: prompt the mobile device to capture video data depicting a subject walking along a predetermined path and/or performing other functional activity(ies) or task(s) (e.g., transitioning from a sitting position to a standing position, or vise versa); process the video data to generate a data object for the subject; determine a plurality of gait features and balance features for the subject from the data object; and determine, using a machine learning model, whether the subject has cognitive impairment, based, at least in part on analysis of the determined plurality of gait features and balance features. The instructions when executed by the processor may cause the processor to further: obtain cognitive task data for the subject while the subject is walking; and analyze the cognitive task data in conjunction with the video data.

The present solution further concerns a non-transitory computer readable medium comprising: a memory having instructions stored thereon to cause a processor to: obtain video data depicting a subject walking along a predetermined path; process the video data to generate a data object for the subject; determine a plurality of gait features and balance features for the subject from the data object; and determine, using a machine learning model, whether the subject has cognitive impairment, based, at least in part on analysis of the determined plurality of gait features and balance features.

The present solution further concerns a computer-implemented method comprising: obtaining, by at least one processor, first video data depicting a subject walking along a predetermined path; obtaining, by the at least one processor, second video data depicting the subject walking along the predetermined path while performing at least one cognitive task; processing the first video data and the second video data to generate, by the at least one processor, a data object for the subject; determining, by the at least one processor, a plurality of gait features and balance features for the subject from the data object; and determining, by the at least one processor and using a machine learning model, whether the subject has cognitive impairment, based, at least in part on analysis of the determined plurality of gait features and balance features. The computer-implemented method may further comprise: determining, by the at least one processor, a first cognitive load corresponding with the first video data and a second cognitive load corresponding with the second video data, wherein a difference between the first cognitive load and the second cognitive load is analyzed to determine whether the subject has the cognitive impairment.

In addition to the machine learning operation described above, the present solution can be implemented using one or more artificial intelligence and machine learning operations. The term “artificial intelligence” can include any technique that enables one or more computing devices or comping systems (i.e., a machine) to mimic human intelligence. Artificial intelligence (AI) includes but is not limited to knowledge bases, machine learning, representation learning, and deep learning. The term “machine learning” is defined herein to be a subset of AI that enables a machine to acquire knowledge by extracting patterns from raw data. Machine learning techniques include, but are not limited to, transformer-based models (e.g., Bidirectional Encoder Representations from Transformers (BERT), Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders and embeddings. The term “deep learning” is defined herein to be a subset of machine learning that enables a machine to automatically discover representations needed for feature detection, prediction, classification, etc., using layers of processing. Deep learning techniques include but are not limited to artificial neural networks or multilayer perceptron (MLP).

An artificial neural network (ANN) is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers such as input layer, an output layer, and optionally one or more hidden layers with different activation functions. An ANN having hidden layers can be referred to as a deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tanh, or rectified linear unit (ReLU) function), and provide an output in accordance with the activation function. Additionally, each node is associated with a respective weight. ANNs are trained with a dataset to maximize or minimize an objective function. In some implementations, the objective function is a cost function, which is a measure of the ANN's performance (e.g., error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for ANNs include but are not limited to backpropagation. It should be understood that an artificial neural network is provided only as an example machine learning model. This disclosure contemplates that the machine learning model can be any supervised learning model, semi-supervised learning model, or unsupervised learning model. Optionally, the machine learning model is a deep learning model. Machine learning models are known in the art and are therefore not described in further detail herein.

A CNN is a type of deep neural network that has been applied, for example, to image analysis applications. Unlike other neural networks, each layer in a CNN has a plurality of nodes arranged in three dimensions (width, height, depth). CNNs can include different types of layers, e.g., convolutional, pooling, and fully-connected (also referred to herein as “dense”) layers. A convolutional layer includes a set of filters and performs the bulk of the computations. A pooling layer is optionally inserted between convolutional layers to reduce the computational power and/or control overfitting (e.g., by down-sampling). A fully-connected layer includes neurons, where each neuron is connected to all of the neurons in the previous layer. The layers are stacked similar to neural networks. GCNNs are CNNs that have been adapted to work on structured datasets such as graphs.

An SVM is a supervised machine learning model that can be used for regression and/or classification tasks and performs well for applications requiring high-dimensional spaces. An SVM is configured to identify a hyperplane, also referred to as an optimal decision boundary, that separates distinct data classes within a given data set.

An LR classifier is a supervised classification model that uses the logistic function to predict the probability of a target, which can be used for classification. LR classifiers are trained with a data set (also referred to herein as a “dataset”) to maximize or minimize an objective function, for example, a measure of the LR classifier's performance (e.g., error such as L1 or L2 loss), during training. This disclosure contemplates that any algorithm that finds the minimum of the cost function can be used. LR classifiers are known in the art and are therefore not described in further detail herein.

The present solution also concerns implementing system (e.g., system 100 of FIG. 1) and methods (e.g., method 200 of FIG. 2) for detection and/or prediction of fall risks and cognitive impairment. The methods comprise: capturing, by a camera (e.g., camera 114 of FIG. 1), a video of a subject (e.g., subject 108 of FIG. 1) performing an activity or task with a cognitive load and without the cognitive load; processing, by a processor (e.g., computing device(s) 102 and/or 112 of FIG. 1, and/or CPU 1906 of FIG. 19), the video to generate a data object for the subject; determining, by the processor or a machine learning model (e.g., machine learning model 1970 of FIG. 19), gait features and balance features for the subject from the data object; performing, by the processor or machine learning model, operations to detect or predict a fall risk and/or cognitive impairment for the subject based on at least the gait features and balance features; and determining, by the processor, a diagnosis for the subject based on the detected or predicted fall risk and/or cognitive impairment.

The present solution also concerns implementing system and methods for detection and/or prediction of fall risks and cognitive impairment. The methods comprise: generating, by a plurality of sensor systems, multimodal sensor data while a subject performs an activity or task with a cognitive load and without the cognitive load; processing, by a processor or a machine learning model, the multimodal sensor data to determine gait features and balance features for the subject; performing, by the processor or machine learning model, operations to detect or predict a fall risk and/or cognitive impairment for the subject based on at least the gait features and balance features; and determining, by the processor, a diagnosis for the subject based on the detected or predicted fall risk and/or cognitive impairment.

The methods can also comprise: controlling a visual stimulation device to provide a visual stimulus to change the cognitive load while the subject is performing the activity or task; and/or tailoring the visual stimulus to a cognitive status of the subject. The visual stimulus can include, but is not limited to, a light pattern, a randomized sequence of colored lights, or a defined sequence of colored lights. The visual stimulation device is at least partially integrated along a path traveled by the subject.

The methods may also comprises controlling, by the processor, electronic medical equipment to facilitate treatment of the subject based on the diagnosis determined for the subject. For example, this may be achieved by: controlling a medical device to increase blood flow to a brain of the subject; reconfiguring parameters of a hearing aid to reduce a rate of cognitive decline in the subject; reconfiguring parameters or settings of a cognitive orthotic system to assist with completing daily tasks by the subject; and/or reconfiguring parameters of a medication dispensing device to increase or decrease a dose of medicine or a timing between medicine dispenses.

The gait features and balance features may be determined by examining how cognitive impairment affects gait with various amounts of cognitive load and analyzing a cost of transitioning from a first cognitive load state to a different second cognitive load state. The gait features can include, but are not limited to, micro temporal features, micro spatial features, micro spatiotemporal features, and macro features. The activity or task may comprise walking a path, sitting in a chair, standing, transitioning from a sitting position to a standing position, and/or transiting from a standing position to a sitting position.

The methods may further comprise: obtaining one or more other videos while one or more individuals perform the activity or task with and without the cognitive load; processing the one or more other videos to extract skeletal body joint data; and/or using the skeletal body joint data to train the machine learning model to identify differences in gait that correlate with cognitive impairment.

The present solution also concerns a system comprising a camera and a processor. The camera is configured to capture a video of a subject performing an activity or task with a cognitive load and without the cognitive load. The processor is configured to: process the video to generate a data object for the subject; determine gait features and balance features for the subject from the data object; detect or predict a fall risk and/or cognitive impairment for the subject based on at least the gait features and balance features; and/or determine a diagnosis for the subject based on the detected or predicted fall risk and/or cognitive impairment. The gait features and balance features may be determined by examining how cognitive impairment affects gait with various amounts of cognitive load and analyzing a cost of transitioning from a first cognitive load state to a different second cognitive load state. The activity or task can include, but is not limited to, walking a path, sitting in a chair, standing, transitioning from a sitting position to a standing position, and/or transiting from a standing position to a sitting position.

The present solution also concerns a system comprising a plurality of sensor systems and a processor. The sensor systems are configured to generate multimodal sensor data while a subject performs activity or task with a cognitive load and without the cognitive load. The processor is configured to: process the multimodal sensor data to determine gait features and balance features for the subject from the data object; detect or predict a fall risk and/or cognitive impairment for the subject based on at least the gait features and balance features; and/or determine a diagnosis for the subject based on the detected or predicted fall risk and/or cognitive impairment

The systems may also comprise a visual stimulation device. In this scenarios, the processor may be further configured to (i) control the visual stimulation device to provide a visual stimulus to change the cognitive load while the subject is performing the activity or task, and/or (ii) tailor the visual stimulus to a cognitive status of the subject. The visual stimulus can include, but is not limited to, a light pattern, a randomized sequence of colored lights, and/or a defined sequence of colored lights.

The systems may also comprise electronic medical equipment. In this scenarios, the processor may be configured to control the electronic medical equipment to facilitate treatment of the subject based on the diagnosis determined for the subject. The electronic medical equipment can include, but is not limited to, a medical device controllable to increase or decrease blood flow to a brain of the subject, a hearing aid having reconfigurable parameters reduce a rate of cognitive decline in the subject, a cognitive orthotic system having reconfigurable parameters or settings to assist with completing daily tasks by the subject, and/or a medication dispensing device having reconfigurable parameters to increase or decrease a dose of medicine or a timing between medicine dispenses.

In some scenarios, another video is captured by the camera while the subject performs the activity or task with and without a cognitive load. The fall risk and/or cognitive impairment is detected or predicted by a machine learning model that was trained to identify differences in gait that correlate with cognitive impairment based on skeletal body joint data extracted from one or more videos of one or more individuals performing the activity or task with and without the cognitive load.

Throughout the description and claims of this specification, the word “comprise” and other forms of the word, such as “comprising” and “comprises,” means including but not limited to, and are not intended to exclude, for example, other additives, segments, integers, or steps. Furthermore, it is to be understood that the terms comprise, comprising, and comprises as they relate to various embodiments, elements, and features of the disclosure also include the more limited embodiments of “consisting essentially of” and “consisting of.”

As used herein, the singular forms “a,” “an,” and “the” include plural referents unless the context clearly dictates otherwise. Thus, for example, reference to a “computing device” includes embodiments having two or more such computing devices unless the context clearly indicates otherwise.

Ranges can be expressed herein as from “about” one particular value and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It should be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

For the terms “for example” and “such as,” and grammatical equivalences thereof, the phrase “and without limitation” is understood to follow unless explicitly stated otherwise.

As used herein, the terms “data,” “content,” “information,” and similar terms may be used interchangeably to refer to data capable of being transmitted, received and/or stored in accordance with embodiments of the present invention.

The described features, advantages and characteristics disclosed herein may be combined in any suitable manner. One skilled in the relevant art will recognize, in light of the description herein, that the disclosed systems and/or methods can be practiced without one or more of the specific features. In other instances, additional features and advantages may be recognized in certain scenarios that may not be present in all instances.

Although the systems and methods have been illustrated and described with respect to one or more implementations, equivalent alterations and modifications will occur to others skilled in the art upon the reading and understanding of this specification and the annexed drawings. In addition, while a particular feature may have been disclosed with respect to only one of several implementations, such feature may be combined with one or more other features of the other implementations as may be desired and advantageous for any given or particular application. Thus, the breadth and scope of the disclosure herein should not be limited by any of the above descriptions. Rather, the scope of the invention should be defined in accordance with the following claims and their equivalents.

What is claimed is:

1. A method for detection and/or prediction of fall risks and cognitive impairment, comprising:
capturing, by a camera, a video of a subject performing an activity or task with a cognitive load and without the cognitive load;
processing, by a processor, the video to generate a data object for the subject;
processing, by a processor or a machine learning model, the data object to determine gait features and balance features for the subject;
performing, by the processor or machine learning model, operations to detect or predict a fall risk and/or cognitive impairment for the subject based on at least the gait features and balance features; and
determining, by the processor, a diagnosis for the subject based on the detected or predicted fall risk and/or cognitive impairment.

2. The method according to claim 1, further comprising controlling a visual stimulation device to provide a visual stimulus to change the cognitive load while the subject is performing the activity or task.

3. The method according to claim 2, further comprising tailoring the visual stimulus to a cognitive status of the subject.

4. The method according to claim 2, wherein the visual stimulus comprises a light pattern, a randomized sequence of colored lights, or a defined sequence of colored lights.

5. The method according to claim 2, wherein the visual stimulation device is at least partially integrated along a path traveled by the subject.

6. The method according to claim 1, further comprising controlling, by the processor, electronic medical equipment to facilitate treatment of the subject based on the diagnosis determined for the subject.

7. The method according to claim 6, wherein said controlling electronic medical equipment comprises controlling a medical device to increase blood flow to a brain of the subject, reconfiguring parameters of a hearing aid to reduce a rate of cognitive decline in the subject, reconfiguring parameters or settings of a cognitive orthotic system to assist with completing daily tasks by the subject, and/or reconfiguring parameters of a medication dispensing device to increase or decrease a dose of medicine or a timing between medicine dispenses.

8. The method according to claim 1, wherein the gait features and balance features are determined by examining how cognitive impairment affects gait with various amounts of cognitive load and analyzing a cost of transitioning from a first cognitive load state to a different second cognitive load state.

9. The method according to claim 1, wherein the gait features comprise micro temporal features, micro spatial features, micro spatiotemporal features, and macro features.

10. The method according to claim 1, wherein the activity or task comprises walking a path, sitting in a chair, standing, transitioning from a sitting position to a standing position, and/or transiting from a standing position to a sitting position.

11. The method according to claim 1, further comprising:
obtaining other sensor data while one or more individuals perform the activity or task with and without the cognitive load;
processing the other sensor data to extract skeletal body joint data; and
using the skeletal body joint data to train the machine learning model to identify differences in gait that correlate with cognitive impairment.

12. A system, comprising:
a camera configured to capture a video of a subject performing activity or task with a cognitive load and without the cognitive load; and
a processor configured to:
process the video to generate a data object for the subject;
determine gait features and balance features for the subject from the data object;
detect or predict a fall risk and/or cognitive impairment for the subject based on at least the gait features and balance features; and
determine a diagnosis for the subject based on the detected or predicted fall risk and/or cognitive impairment.

13. The system according to claim 12, wherein the system further comprises a visual stimulation device and the processor is further configured to control the visual stimulation device to provide a visual stimulus to change the cognitive load while the subject is performing the activity or task.

14. The system according to claim 13, wherein the processor is further configured to tailor the visual stimulus to a cognitive status of the subject.

15. The system according to claim 14, wherein the visual stimulus comprises a light pattern, a randomized sequence of colored lights, or a defined sequence of colored lights.

16. The system according to claim 13, wherein the system further comprises electronic medical equipment and the processor is further configured to control the electronic medical equipment to facilitate treatment of the subject based on the diagnosis determined for the subject.

17. The system according to claim 16, wherein the electronic medical equipment comprises a medical device controllable to increase or decrease blood flow to a brain of the subject, a hearing aid having reconfigurable parameters reduce a rate of cognitive decline in the subject, a cognitive orthotic system having reconfigurable parameters or settings to assist with completing daily tasks by the subject, and/or a medication dispensing device having reconfigurable parameters to increase or decrease a dose of medicine or a timing between medicine dispenses.

18. The system according to claim 13, wherein the gait features and balance features are determined by examining how cognitive impairment affects gait with various amounts of cognitive load and analyzing a cost of transitioning from a first cognitive load state to a different second cognitive load state.

19. The system according to claim 13, wherein:
other sensor data is obtained while the subject performs the activity or task with and without a cognitive load; and
the fall risk and/or cognitive impairment is detected or predicted by a machine learning model that was trained to identify differences in gait that correlate with cognitive impairment based on skeletal body joint data extracted from the other sensor data.
